(19) United States
(12) Patent Application Publication
(10) Pub. No.: US 2023/0235087 A1
(43) Pub. Date: Jul. 27, 2023

(54) ANTI-GD2 SADA CONJUGATES AND USES THEREOF

(71) Applicant: MEMORIAL SLOAN KETTERING CANCER CENTER, New York, NY (US)

(72) Inventors: Brian H. Santich, New York, NY (US); Nai-Kong V. Cheung, New York, NY (US); Mahiuddin Ahmed, New York, NY (US)

(21) Appl. No.: 17/999,786

(22) PCT Filed: May 26, 2021
(86) PCT No.: PCT/US2021/034230

§ 371 Date: Nov. 23, 2022

Related U.S. Application Data
(60) Provisional application No. 63/030,591, filed on May 27, 2020.

(51) Int. Cl. C07K 16/44; A61P 39/04; C07K 16/30; A61K 51/04; A61P 35/00

(52) U.S. Cl. CPC C07K 16/44; A61P 39/04; C07K 16/3084; A61K 51/0482; A61P 35/00; A61K 2039/505

(57) ABSTRACT

The present technology relates to the use of protein conjugates including a self-assembly and disassembly (SADA) polypeptide and a GD2-specific antigen binding domain for preventing or mitigating off-target tissue toxicity, such as brain, kidney, and/or myeloid damage, in a subject undergoing targeted alpha radioimmunotherapy. Also disclosed herein are pretargeted radioimmunotherapy (PRIT) methods that improve the durability of the anti-GD2-SADA conjugate anti-tumor response in vivo.

### CROSS-REFERENCE TO RELATED APPLICATIONS

This application is a U.S. National Stage Application of PCT/US2021/034230, filed May 26, 2021, which claims the benefit of and priority to U.S. Provisional Patent Application No. 63/030,591, filed May 27, 2020, the entire contents of which are incorporated herein by reference.

### STATEMENT OF GOVERNMENT SUPPORT

This invention was made with government support under CA008748, awarded by the National Cancer Institute/National Institutes of Health. The government has certain rights in the invention.

### TECHNICAL FIELD

The present technology relates to methods employing conjugates that include a self-assembly and disassembly (SADA) polypeptide and a GD2-specific antigen binding domain. In particular, the present disclosure provides methods for preventing or mitigating off-target tissue toxicity, such as brain, kidney, and/or myeloid damage, in a subject undergoing targeted alpha radioimmunotherapy. Also disclosed herein are pretargeted radioimmunotherapy (PRIT) methods that improve the durability of the anti-tumor response of anti-GD2-SADA protein conjugates in vivo.

### SEQUENCE LISTING

The instant application contains a Sequence Listing which has been submitted electronically in ASCII format and is hereby incorporated by reference in its entirety. Said ASCII copy, created on Jul. 2, 2021, is named 115872-2219_SL.txt and is 96,089 bytes in size.

### BACKGROUND

The following description of the background of the present technology is provided simply as an aid in understanding the present technology and is not admitted to describe or constitute prior art to the present technology.

Metastatic disease remains a major barrier to cancer cures. While localized disease can be controlled by surgery or radiation therapy, widespread, distant and occult metastases require systemic therapies. Yet, many of these treatments have unintended dose-limiting toxicities to vital organs due to poor therapeutic indices (TI, the ratio of cumulative tumor uptake to cumulative normal tissue uptake) (Lin, A. et al., Sci Transl Med 11 (2019)). Currently, over 90% of clinical trials fail to receive FDA approval (Dowden, H. & Munro, J. Nature Reviews Drug Discovery 18, 495-496 (2019)), with a significant number due to dose-limiting renal, hepatic or myelotoxicities. For instance, if a therapeutic is too small (<70 kDa) and filtered through the renal glomeruli, either larger doses or extended dosing regimens are necessary to overcome the short serum half-life, which is associated with the accompanying shortcomings of excessive cost, logistics, and increased risk of organ toxicity. See, e.g., Pinzani, V. et al., Cancer Chemoth Pharm 35, 1-9 (1994). Even with tumor-specific targets, conventional 1-step delivery systems, such as antibody drug conjugates (ADC) or radiolabeled immunoglobulin G (IgG) proteins typically have TI below 10:1, and are dosed limited by toxicities to kidneys, liver or bone marrow. Accordingly, the off-target effects of systemic cytotoxic therapy present major hurdles to cancer cures, particularly in children for whom the genomic, physical and intellectual consequences can be severe and long-lasting.

Thus, there is an on-going need for agents that have effective kinetic and/or pharmacological properties with reduced or without associated toxicities.

### SUMMARY OF THE PRESENT TECHNOLOGY

In one aspect, the present disclosure provides a method for reducing or mitigating alpha-radioimmunotherapy-associated toxicity in a subject in need thereof comprising administering to the subject an effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; and administering to the subject an effective amount of a DOTA hapten comprising an alpha particle-emitting isotope, wherein the DOTA hapten is configured to bind to the anti-GD2 SADA conjugate. In certain embodiments, the subject has received or is receiving one or more cycles of alpha-radioimmunotherapy. Examples of alpha particle-emitting isotopes include, but are not limited to, ²¹³Bi, ²¹¹At, ²²⁵Ac, ¹⁵²Dy, ²¹²Bi, ²²³Ra, ²¹⁹Rn, ²¹⁵Po ²¹¹Bi, ²²Fr, ²¹⁷At, or ²⁵⁵Fm. The alpha-radioimmunotherapy-associated toxicity may be toxicity to one or more organs selected from the group consisting of brain, kidney, bladder, liver, bone marrow and spleen. In some embodiments, the subject is human.

In another aspect, the present disclosure provides a method for increasing the efficacy of beta-radioimmunotherapy in a subject in need thereof comprising (a) administering to the subject an effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 48 hours after administration of the anti-GD2 SADA conjugate, wherein the DOTA hapten (i) comprises a beta particle-emitting isotope, and (ii) is configured to bind to the anti-GD2 SADA conjugate; (c) administering to the subject a second dose of the DOTA hapten about 24 hours after administration of the first dose of the DOTA hapten; and (d) administering to the subject a third dose of the DOTA hapten about 24 hours after administration of the second dose of the DOTA hapten. In some embodiments, the radiolabeled-DOTA hapten are administered without further administration of the anti-GD2 SADA conjugate of the present technology. In other embodiments, the method further comprises repeating steps (a)-(d) for at least 1, 2, 3, 4, 5, 6, 7, 8, 9, 10 or more additional cycles. In some embodiments, the subject is human.

In yet another aspect, the present disclosure provides a method for increasing the efficacy of beta-radioimmunotherapy in a subject in need thereof comprising (a) administering to the subject a first effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 48 hours after administration of the first effective amount of the anti-GD2 SADA conjugate, wherein the DOTA hapten (i) comprises a beta particle-emitting isotope, and (ii) is configured to bind to the anti-GD2 SADA conjugate; (c) administering to the subject a second effective amount of the anti-GD2 SADA conjugate about 7 days after administration of the first effective amount of the anti-GD2 SADA conjugate; (d) administering to the subject a second dose of the DOTA hapten about 48 hours after administration of the second effective amount of the anti-GD2 SADA conjugate; (e) administering to the subject a third effective amount of the anti-GD2 SADA conjugate about 7 days after administration of the second effective amount of the anti-GD2 SADA conjugate; and (f) administering to the subject a third dose of the DOTA hapten about 48 hours after administration of the third effective amount of the anti-GD2 SADA conjugate. In some embodiments, the subject is human.

Additionally or alternatively, in some embodiments of the methods disclosed herein, the first, second, and third doses of the DOTA hapten are identical. In other embodiments of the methods disclosed herein, any two of the first, second, and third doses of the DOTA hapten may be identical. In certain embodiments of the methods disclosed herein, the first, second, and third doses of the DOTA hapten are different. In any of the preceding embodiments of the methods disclosed herein, the beta particle-emitting isotope is 86Y ⁹⁰Y, ⁸⁹Sr, ¹⁶⁵Dy, ¹⁸⁶Re, ¹⁸⁸Re, ¹⁷⁷Lu, or ⁶⁷Cu.

In one aspect, the present disclosure provides a method for treating a GD2-associated cancer in a subject in need thereof comprising (a) administering to the subject an effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 48 hours after administration of the anti-GD2 SADA conjugate, wherein the DOTA hapten (i) comprises a beta particle-emitting isotope or an alpha particle-emitting isotope, and (ii) is configured to bind to the anti-GD2 SADA conjugate; (c) administering to the subject a second dose of the DOTA hapten about 24 hours after administration of the first dose of the DOTA hapten; and (d) administering to the subject a third dose of the DOTA hapten about 24 hours after administration of the second dose of the DOTA hapten. In some embodiments, the radiolabeled-DOTA hapten are administered without further administration of the anti-GD2 SADA conjugate of the present technology. In other embodiments, the method further comprises repeating steps (a)-(d) for at least 1, 2, 3, 4, 5, 6, 7, 8, 9, 10 or more additional cycles. In some embodiments, the subject is human.

In another aspect, the present disclosure provides a method for treating a GD2-associated cancer in a subject in need thereof comprising (a) administering to the subject a first effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 48 hours after administration of the first effective amount of the anti-GD2 SADA conjugate, wherein the DOTA hapten (i) comprises a beta particle-emitting isotope or an alpha particle-emitting isotope, and (ii) is configured to bind to the anti-GD2 SADA conjugate; (c) administering to the subject a second effective amount of the anti-GD2 SADA conjugate about 7 days after administration of the first effective amount of the anti-GD2 SADA conjugate; (d) administering to the subject a second dose of the DOTA hapten about 48 hours after administration of the second effective amount of the anti-GD2 SADA conjugate; (e) administering to the subject a third effective amount of the anti-GD2 SADA conjugate about 7 days after administration of the second effective amount of the anti-GD2 SADA conjugate; and (f) administering to the subject a third dose of the DOTA hapten about 48 hours after administration of the third effective amount of the anti-GD2 SADA conjugate. In some embodiments, the subject is human.

Additionally or alternatively, in some embodiments of the methods disclosed herein, the first, second, and third doses of the DOTA hapten are identical. In other embodiments of the methods disclosed herein, any two of the first, second, and third doses of the DOTA hapten may be identical. In certain embodiments of the methods disclosed herein, the first, second, and third doses of the DOTA hapten are different. Examples of the beta particle-emitting isotope include ⁸⁶y ⁹⁰Y, ⁸⁹Sr, ¹⁶⁵Dy, ¹⁸⁶Re, ¹⁸⁸Re, ¹⁷⁷Lu, or ⁶⁷Cu. Examples of the alpha particle-emitting isotope include ²¹³Bi, ²¹¹At, ²²⁵Ac, ¹⁵²Dy, ²¹²Bi, ²²³Ra, ²¹⁹R ²¹⁵Po, ²¹¹Bi, ²²Fr, ²¹⁷At, or ²⁵⁵Fm.

In any and all embodiments of the methods disclosed herein, the subject suffers from or is diagnosed as having a GD2-associated cancer, such as neuroblastoma, melanoma, soft tissue sarcoma, brain tumor, osteosarcoma, small-cell lung cancer, retinoblastoma, liposarcoma, fibrosarcoma, malignant fibrous histiocytoma, leimyosarcoma, breast cancer, or spindle cell sarcoma.

In any of the above embodiments of the methods disclosed herein, the DOTA hapten is selected from the group consisting of DOTA, Proteus-DOTA, DOTA-Bn, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, and Ac-D-Lys(DOTA)-D-Tyr-D-Lys(DOTA)-D-Lys(Tscg-Cys)-NH₂.

Additionally or alternatively, in some embodiments of the methods disclosed herein, the administration of the anti-GD2 SADA conjugate results in decreased renal apoptosis in the subject compared to a GD2-associated cancer patient that has been treated with an anti-DOTA×anti-GD2 IgG-scFv-BsAb. In certain embodiments of the methods described herein, administration of the anti-GD2 SADA conjugate results in reduced immunogenicity in the subject compared to a GD2-associated cancer patient that has been treated with an anti-DOTA×anti-GD2 IgG-scFv-BsAb. Additionally or alternatively, in some embodiments of the methods disclosed herein, administration of the anti-GD2 SADA conjugate results in decreased severity of ovarian atrophy in the subject compared to a GD2-associated cancer patient that has been treated with an anti-DOTA×anti-GD2 IgG-scFv-BsAb. In some embodiments of the methods disclosed herein, administration of the anti-GD2 SADA conjugate results in prolonged remission in the subject compared to a GD2-associated cancer patient that has been treated with an anti-DOTA×anti-GD2 IgG-scFv-BsAb. In any of the preceding embodiments of the methods described herein, the anti-DOTA×anti-GD2 IgG-scFv-BsAb comprises (a) a GD2-specific antigen binding domain comprising a heavy chain variable domain (V_{H}) sequence and a light chain variable domain (V_{L}) sequence of SEQ ID NO: 1 and SEQ ID NO: 5, respectively, and (b) a DOTA-specific antigen binding domain comprising a heavy chain variable domain (V_{H}) sequence of SEQ ID NO: 9 or SEQ ID NO: 17, and a light chain variable domain (V_{L}) sequence of SEQ ID NO: 13 or SEQ ID NO: 18.

In any and all embodiments of the methods disclosed herein, administration of the anti-GD2 SADA conjugate results in decreased renal apoptosis, decreased severity of ovarian atrophy, and/or prolonged remission in the subject compared to a control GD2-associated cancer patient that does not receive the anti-GD2 SADA conjugate.

In any and all embodiments of the methods disclosed herein, the GD2-specific antigen binding domain of the anti-GD2 SADA conjugates comprise a heavy chain variable domain (V_{H}) sequence and a light chain variable domain (V_{L}) sequence of SEQ ID NO: 1 and SEQ ID NO: 5, respectively. Additionally or alternatively, in some embodiments, the DOTA-specific antigen binding domain of the anti-GD2 SADA conjugates comprise a heavy chain variable domain (V_{H}) sequence of SEQ ID NO: 9 or SEQ ID NO: 17, and a light chain variable domain (V_{L}) sequence of SEQ ID NO: 13 or SEQ ID NO: 18.

In any of the preceding embodiments of the methods disclosed herein, the V_{H} domain sequence and the V_{L} domain sequence in the GD2-specific antigen binding may be linked via an intra-peptide linker. Additionally or alternatively, in some embodiments, the sequence of the intra-peptide linker between the V_{H} domain sequence and the V_{L} domain sequence in the GD2-specific antigen binding domain is any one of SEQ ID NOs: 19-21.

In any and all embodiments of the methods disclosed herein, the V_{H} domain sequence and the V_{L} domain sequence in the DOTA-specific antigen binding may be linked via an intra-peptide linker. Additionally or alternatively, in some embodiments, the sequence of the intra-peptide linker between the V_{H} domain sequence and the V_{L} domain sequence in the DOTA-specific antigen binding domain is any one of SEQ ID NOs: 19-21.

In any and all embodiments of the methods of the present technology, the GD2-specific antigen binding domain and the DOTA-specific antigen binding domain may be linked via an intra-peptide linker. Additionally or alternatively, in some embodiments, the sequence of the intra-peptide linker between the GD2-specific antigen binding domain and the DOTA-specific antigen binding domain is any one of SEQ ID NOs: 19-21.

In certain embodiments, the anti-GD2 SADA conjugate of the present technology comprises a first polypeptide chain, wherein the first polypeptide chain comprises in the N-terminal to C-terminal direction: (i) the V_{L} sequence of SEQ ID NO: 5; (ii) a flexible peptide linker comprising the amino acid sequence of any one of SEQ ID NOs: 19-21; (iii) the V_{H} sequence of SEQ ID NO: 1; (iv) a flexible peptide linker comprising the amino acid sequence of any one of SEQ ID NOs: 19-21; (v) the V_{H} sequence of SEQ ID NO: 9 or SEQ ID NO: 17; (vi) a flexible peptide linker comprising the amino acid sequence of any one of SEQ ID NOs: 19-21; (vii) the V_{L} sequence of SEQ ID NO: 13 or SEQ ID NO: 18; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In some embodiments, the anti-GD2 SADA conjugate of the present technology comprises a first polypeptide chain, wherein the first polypeptide chain comprises in the N-terminal to C-terminal direction: (i) the V_{L} sequence of SEQ ID NO: 5; (ii) a flexible peptide linker comprising the amino acid sequence of any one of SEQ ID NOs: 19-21; (iii) the V_{H} sequence of SEQ ID NO: 1; (iv) a flexible peptide linker comprising the amino acid sequence of any one of SEQ ID NOs: 19-21; (v) the V_{L} sequence of SEQ ID NO: 13 or SEQ ID NO: 18; (vi) a flexible peptide linker comprising the amino acid sequence of any one of SEQ ID NOs: 19-21; (vii) the V_{H} sequence of SEQ ID NO: 9 or SEQ ID NO: 17; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In other embodiments, the anti-GD2 SADA conjugate of the present technology comprises a first polypeptide chain, wherein the first polypeptide chain comprises in the N-terminal to C-terminal direction: (i) the V_{H} sequence of SEQ ID NO: 1; (ii) a flexible peptide linker comprising the amino acid sequence of any one of SEQ ID NOs: 19-21; (iii) the V_{L} sequence of SEQ ID NO: 5; (iv) a flexible peptide linker comprising the amino acid sequence of any one of SEQ ID NOs: 19-21; (v) the V_{H} sequence of SEQ ID NO: 9 or SEQ ID NO: 17; (vi) a flexible peptide linker comprising the amino acid sequence of any one of SEQ ID NOs: 19-21; (vii) the V_{L} sequence of SEQ ID NO: 13 or SEQ ID NO: 18; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In some embodiments, the anti-GD2 SADA conjugate of the present technology comprises a first polypeptide chain, wherein the first polypeptide chain comprises in the N-terminal to C-terminal direction: (i) the V_{H} sequence of SEQ ID NO: 1; (ii) a flexible peptide linker comprising the amino acid sequence of any one of SEQ ID NOs: 19-21; (iii) the V_{L} sequence of SEQ ID NO: 5; (iv) a flexible peptide linker comprising the amino acid sequence of any one of SEQ ID NOs: 19-21; (v) the V_{L} sequence of SEQ ID NO: 13 or SEQ ID NO: 18; (vi) a flexible peptide linker comprising the amino acid sequence of any one of SEQ ID NOs: 19-21; (vii) the V_{H} sequence of SEQ ID NO: 9 or SEQ ID NO: 17; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In any and all of the preceding embodiments of the methods disclosed herein, the amino acid sequence of the GD2 SADA conjugate is selected from among SEQ ID NOs: 22-35 or 38-39.

Also disclosed herein are kits comprising at least one anti-GD2 SADA conjugate of the present technology, a DOTA hapten, and instructions for using the same in alpha- or beta-radioimmunotherapy (e.g., PRIT).

### BRIEF DESCRIPTION OF THE DRAWINGS

FIGS. 1A-1E show an overview of multi-step payload delivery and anti-GD2/anti-DOTA SADA conjugate (a.k.a. SADA-BsAb) activity in vitro. FIG. 1A shows a schematic of 4 different payload delivery strategies. Tumor-specific domains and DOTA-specific domains are indicated. The concentration of payload in the blood over time, the concentration of payload in the tumor and the concentration of non-payload antibody in the blood are also indicated. The area of each curve (AUC) represents the relative exposure of each. FIG. 1B shows a schematic of a representative anti-GD2/anti-DOTA SADA conjugate. Each monomer is made of 3 domains: an anti-tumor domain, an anti-DOTA domain and a SADA domain, from N-terminus to C-terminus, respectively. SADA domains self-assemble into tetramers (220 kDa) but also disassemble into monomers (55 kDa). FIG. 1C shows a representative SEC-HPLC chromatogram of anti-GD2/anti-DOTA P53noHIS-SADA conjugate (a.k.a. P53-SADA-BsAb noHIS; (SEQ ID NO: 22)) with high and low molecular weight impurities indicated. FIG. 1D shows normalized GD2 binding kinetics of anti-GD2/anti-DOTA P53-SADA conjugate (a.k.a. P53-SADA-BsAb; (SEQ ID NO: 27)) and anti-GD2/anti-DOTA P63-SADA conjugate (a.k.a. P63-SADA-BsAb; (SEQ ID NO: 28)) compared to anti-GD2/anti-DOTA IgG-scFv-BsAb (a.k.a. IgG-scFv-BsAb), as measured by surface plasmon resonance (SPR). For each curve maximum binding was normalized to 100. FIG. 1E shows representative cell binding analysis of anti-GD2/anti-DOTA P53-SADA conjugate (a.k.a. P53-SADA-BsAb LS; (SEQ ID NO: 23)) and anti-GD2/anti-DOTA P63-SADA conjugate (a.k.a. P63-SADA-BsAb LS; (SEQ ID NO: 24)) by flow cytometry. Each curve represents the fluorescence histogram of one BsAb or a control BsAb (irrelevant tumor specificity).

FIGS. 2A-2D show in vivo pharmacokinetics and biodistribution of SADA-BsAbs of the present technology. FIG. 2A shows serum clearance kinetics of the tested SADA-BsAbs. Tumor-free mice (n=3) were injected with 131I-radiolabeled P53-SADA-BsAb LS (SEQ ID NO: 23) or P63-SADA-BsAb LS (SEQ ID NO: 24) and serially bled over 48 hours. The graph represents the amount of remaining BsAb per unit of blood normalized to peak concentration (0.5 hour). FIG. 2B shows the relationship between administered dose and tissue uptake using 2-step SADA-PRIT. Mice (n=5) were administered P53-SADA-BsAb (SEQ ID NO: 27) (1.25 nmol) and one of 3 doses of DOTA[¹⁷⁷Lu]: 3.7, 18.5 or 37 MBq (20, 100 or 200 pmol, respectively). The level of DOTA payload in the tumor, kidney, and blood are indicated. The therapeutic index between tumor and blood at each dose is also shown. Tissue uptake was normalized to pmol of DOTA[¹⁷⁷Lu] per gram of tissue. FIGS. 2C-2D show an exemplary schematic and PET/CT images using the SADA-BsAbs of the present technology, respectively. As depicted in the schematic, mice (n=1-2) were injected with P53-SADA-BsAb (SEQ ID NO: 27) or IgG-scFv-BsAb (with and without clearing agents) followed by DOTA[⁸⁶Y] (downward arrows correspond to each injection). Mice were imaged for 30 minutes (upward arrow) 18 hours after the administration of DOTA. The representative images were normalized using the same scale. Arrows point to the subcutaneous tumor (left panel) or the bladder (middle panel).

FIGS. 3A-3B show the immunogenicity of the SADA-BsAb of the present technology. Mice (n=5) were immunized with P53-SADA-BsAb (SEQ ID NO: 27) or IgG-scFv-BsAb and bled 4 weeks later. Mice received a follow up dose of BsAb and were bled again 4 weeks later. Anti-BsAb titers were measured by ELISA and normalized to a monoclonal anti-BsAb standard. Statistical significances were calculated using a Mann Whitney test. **P=0.0079 for IgG-scFv-BsAb compared to P53-SADA-BsAb.

FIG. 4A shows a schematic of a neuroblastoma xenograft treatment model (left) and mean tumor responses (right). One dose of BsAb (SEQ ID NO: 27 or (SEQ ID NO: 28) (1.25 nmol, triangle) was followed by one dose of DOTA[¹⁷⁷Lu] (18.5 MBq, 100 pmol, star) 48 hours later, once per week for 3 weeks. Each solid line represents one treatment group (n=10). The dotted black line represents no measurable tumor, and the boxed hexagon represents the tumor implantation. Tumor averages were calculated until at least one mouse had to be euthanized. Data are shown as means±standard deviation. FIG. 4B shows individual tumor responses for each experimental group. Each solid line represents tumors from a single mouse, and the dashed line represents the group average. FIG. 4C shows progression-free survival analysis for each experimental group. Tumors were considered “progressing” when their volume reached 500 mm³. Mice were censored if they were sacrificed for histological analysis but were otherwise healthy at the time. FIG. 4D shows graphical representation of organ pathologies observed in treated mice. Each bar represents one treatment group and each graph represents analysis of either ovaries (left) or bladders (right). Y-axis values represent the percentage of analyzed mice displaying the toxicity. Grade 4, Grade 3, and Grade 2 toxicities vs. normal phenotype are indicated. n=9 for IgG-scFv-BsAb, P53-SADA-BsAb (SEQ ID NO: 27) and control mice (age-matched non-tumor littermates), and n=6 for P63-SADA-BsAb (SEQ ID NO: 28). Statistical significances were calculated by two-way analysis of variance (ANOVA) with Tukey correction or Log-rank (Mantel-Cox) test. ****P<0.0001 between DOTA[¹⁷⁷Lu] alone and P53-SADA-BsAb, P63-SADA-BsAb or IgG-scFv-BsAb.

FIG. 5A shows a schematic of a DOTA[¹⁷⁷Lu] neuroblastoma xenograft treatment model (left) and mean tumor responses (right). One dose of BsAb (SEQ ID NO: 27) (1.25 nmol, triangle) was followed by one dose of DOTA[¹⁷⁷Lu] (55.5 MBq, 300 pmol, star) 48 hours later, once per week for 3 weeks. Each solid line represents one treatment group (n=5). The dotted black line represents no measurable tumor, and the boxed hexagon represents the tumor implantation. Tumor averages were calculated until at least one mouse had to be euthanized. Data are shown as means±standard deviation. FIG. 5B shows progression-free survival analysis for each experimental group. Tumors were considered “progressing” when their volume reached 500 mm³. Mice were censored if they were sacrificed for histological analysis but were otherwise healthy at the time.

FIG. 5C shows a schematic of a Proteus[²²⁵Ac] neuroblastoma xenograft treatment model (left) and mean tumor responses (right). The structure of the Proteus DOTA-hapten is described in WO2019/010299. Proteus-DOTA was synthesized by mixing two bifunctional DOTA chelators: commercial 2,2′,2″-(10-(17-amino-2-oxo-6,9,12,15-tetraoxa-3-azaheptadecyl)-1,4,7,10-tetraazacyclododecane-1,4,7-triyl)triacetic acid (amine-PEG4-DOTA) and the non-radioactive lutetium-complex of 2-(4-isothiocyanatobenzyl)-1,4,7,10-tetraazacyclododecane-tetraacetic acid (p-SCN-Bn-DOTA Lu³⁺ complex) prepared from commercial p-SCN-Bn-DOTA and LuCl₃·6 H₂O. One dose of BsAb (SEQ ID NO: 27) (1.25 nmol, triangle) was followed by one dose of Proteus[²²⁵Ac] (37 kBq, 2.4 nmol, star) 48 hours later. Each solid line represents one treatment group (n=5). The dotted black line represents no measurable tumor, and the boxed hexagon represents the tumor implantation. Tumor averages were calculated until at least one mouse had to be euthanized. Data are shown as means±standard deviation. FIG. 5D shows progression-free survival analysis for each experimental group. Tumors were considered “progressing” when their volume reached 500 mm³. Mice were censored if they were sacrificed for histological analysis but were otherwise healthy at the time. Statistical significances were calculated by two-way analysis of variance (ANOVA) with Tukey correction or Log-rank (Mantel-Cox) test. **P=0.034, ****P<0.0001 between DOTA[¹⁷⁷Lu] alone or Proteus[²²⁵Ac] alone and P53-SADA-BsAb (SEQ ID NO: 27) or IgG-scFv-BsAb.

FIG. 6A shows a schematic of Proteus[²²⁵Ac] small-cell lung cancer patient-derived xenograft (PDX) treatment model (left) and mean tumor responses (right). One dose of BsAb (SEQ ID NO: 27) (1.25 nmol, triangle) was followed by one dose of Proteus[²²⁵Ac] (37 kBq, 621 pmol, star) 48 hours later. Each line represents one treatment group (n=5). The dotted black line represents no measurable tumor, and the boxed hexagon represents the tumor implantation. Tumor averages were calculated until at least one mouse had to be euthanized. Data are shown as means±standard deviation. FIG. 6B shows individual tumor responses for each experimental group. Each solid line represents tumors from a single mouse, and the dashed line represents the group average. FIG. 6C progression-free survival analysis for each experimental group. Tumors were considered “progressing” when their volume reached 500 mm³. No mice died unexpectedly in this study. Statistical significances were calculated by two-way analysis of variance (ANOVA) with Sidak correction or Log-rank (Mantel-Cox) test. ****P<0.0001 between Proteus[²²⁵Ac] alone and P53-SADA-BsAb.

FIG. 7A shows a schematic of a neuroblastoma xenograft treatment model (left) and mean tumor responses (right). One dose of BsAb (SEQ ID NO: 28) (1.25 nmol, triangle) was followed by 3 subsequent doses of DOTA[¹⁷⁷Lu] (18.5 MBq, 100 pmol, vertical bar) at 48 hrs, 72 hrs and 96 hours after administration of the BsAb, once per week for 3 weeks. Each solid line represents one treatment group (n=5-10). The dotted black line represents no measurable tumor, and the boxed hexagon represents the tumor implantation. Tumor averages were calculated until at least one mouse had to be euthanized. Data are shown as means±standard deviation. FIG. 7B shows individual tumor responses for each experimental group. Each solid line represent tumors from a single mouse, and the dashed line represents the group average. FIG. 7C shows progression-free survival analysis for each experimental group. Tumors were considered “progressing” when their volume reached 500 mm³. Mice were censored if they were sacrificed for histological analysis but were otherwise healthy at the time. FIG. 7D shows a graphical representation of organ pathologies observed in treated mice. Each bar represents one treatment group and each graph represents analysis of either ovaries (left) or bladders (right). Y-axis values represent the percentage of analyzed mice displaying the toxicity. Grade 3 toxicity and no pathologies (normal) are indicated. n=6 for 3×-3×, n=2 for 1×-3× and 2×-6×, and n=9 for the controls (age-matched non-tumor littermates). Statistical significances were calculated by two-way analysis of variance (ANOVA) with Tukey correction or Log-rank (Mantel-Cox) test. ***P<0.0005, ****P<0.0001 between DOTA[¹⁷⁷Lu] alone and 3×-3×, lx-3× or 2×-6×.

FIG. 8A shows an exemplary hematology analysis of DOTA[¹⁷⁷Lu] treated mice in each of the following experimental groups: P53-SADA-BsAb (SEQ ID NO: 27), P63-SADA-BsAb (SEQ ID NO: 28), IgG-scFv-BsAb, control group (DOTA[¹⁷⁷Lu] Alone), and age-matched non-tumor littermates. White blood cell (WBC, left), Red blood cell (RBC, center), and platelet (PLT, right) counts from mouse blood are shown. All mice were bled 14 days after the first dose of DOTA[¹⁷⁷Lu]. Each symbol refers to a single mouse (n=10). The black dotted line refers to mean values from age-matched mice irradiated with 300 cGy of total body irradiation (TBI) on day 0, and the grey bar represents the one standard deviation above and below this mean. FIG. 8B shows FLT3L levels in the plasma of treated mice. All mice were bled 21 days after the first dose of DOTA[¹⁷⁷Lu]. Each symbol refers to a single mouse (n=10). FIG. 8C shows weight change in treated mice. Weights were monitored at least once per week and normalized to each individual mouse's pre-treatment weight. Each solid line represents one treatment group (n=10). The dotted black line represents 10% increases or decreases in weight. Average weights were calculated until at least one mouse had to be euthanized. Data are shown as means±standard deviation.

FIG. 9A shows an exemplary hematology analysis of DOTA[¹⁷⁷Lu] treated mice in each of the following experimental groups: the P63-SADA-BsAb (SEQ ID NO: 28) 3×-3× regimen, P63-SADA-BsAb (SEQ ID NO: 28) lx-3× regimen, P63-SADA-BsAb (SEQ ID NO: 28) 2×-6×regimen, the control group (DOTA[¹⁷⁷Lu] Alone), and age-matched non-tumor littermates. White blood cell (WBC, left), Red blood cell (RBC, center), and platelet (PLT, right) counts from mouse blood are shown. All mice were bled 14 days after the first dose of DOTA[¹⁷⁷Lu]. Each symbol refers to a single mouse (n=5-10). The black dotted line refers to mean values from age-matched mice irradiated with 300 cGy of total body irradiation (TBI) on day 0. The grey bar represents the mean±one standard deviation. FIG. 9B shows FLT3L levels in the plasma of treated mice. All mice were bled 21 days after the first dose of DOTA[¹⁷⁷Lu]. Each symbol refers to a single mouse (n=10). FIG. 9C shows weight change in treated mice. Weights were monitored at least once per week and normalized to each individual mouse's pre-treatment weight. Each solid line represents one treatment group (n=10). The dotted black line represents 10% increases or decreases in weight. Average weights were calculated until at least one mouse had to be euthanized. Data are shown as means±standard deviation.

FIG. 10 shows representative H&E staining of ovaries from treated nude mice. Normal ovary (left, littermate control), grade 3 atrophied ovary (center, P53-SADA-BsAb (SEQ ID NO: 27)) and grade 4 atrophied ovary (right, IgG-scFv-BsAb). Mice were sacrificed between day 110 and day 230 after treatment start.

FIG. 11A shows individual DOTA[¹⁷⁷Lu] tumor responses in a neuroblastoma PDX treatment model treated with P53-SADA-BsAb (SEQ ID NO: 27). Each solid line represent tumors from a single mouse, and the dashed line represents the group average. FIG. 11B shows a graphical representation of bladder pathologies observed in treated mice. Each bar represents one treatment group (n=5). Y-axis values represent the percentage of analyzed mice displaying the toxicity. Grade 4 toxicity, grade 3 toxicity, grade 2 toxicity and no pathologies (normal) are indicated. FIG. 11C shows individual Proteus[²²⁵Ac] tumor responses in a neuroblastoma model treated with P53-SADA-BsAb (SEQ ID NO: 27), where each line represent tumors from a single mouse, and the dashed line represents the group average. FIG. 11D shows a graphical representation of kidney pathologies observed in treated mice. All pathologies were measured as the number of observations per 10-consecutive fields, beginning with the field containing the most pathologies. Each group (x-axis) represents one treatment group or age-matched littermate control, and each individual scatter plot represents a different stain for kidney damage. Tubular proteinosis, epithelial cell apoptosis, Cleaved Caspase 3 (CC-3) positive cells, and Terminal deoxynucleotidyl transferase dUTP nick end labeling (TUNEL) positive cells are depicted.

FIG. 12A shows an exemplary hematology analysis of DOTA[¹⁷⁷Lu] treated mice in a neuroblastoma model treated with P53-SADA-BsAb (SEQ ID NO: 27). White blood cell (WBC, left), Red blood cell (RBC, center), and platelet (PLT, right) counts from mouse blood are shown. All mice were bled 14 days after the first dose of DOTA[177Lu]. Each symbol refers to a single mouse (n=5). FIG. 12B shows representative H&E staining of bladders from treated mice. Normal bladder (left, littermate control), grade 2 bladder (center left, IgG-scFv-BsAb), grade 3 multifocal bladder (center right, P53-SADA-BsAb) and grade 4 diffuse bladder (right, P53-SADA-BsAb) are shown. Mice were sacrificed at day 120 after treatment initiation.

FIG. 13A shows an exemplary hematology analysis of Proteus[²²⁵Ac] treated mice in a neuroblastoma model treated with P53-SADA-BsAb (SEQ ID NO: 27). White blood cell (WBC, left), Red blood cell (RBC, center), and platelet (PLT, right) counts from mouse blood are shown. All mice were bled 14 days after the first dose of Proteus[²²⁵Ac]. Each symbol refers to a single mouse (n=5). FIG. 13B show representative images of kidneys from IgG-scFv-BsAb treated mice. Cleaved Caspase 3 (CC-3) positive kidney (left), terminal deoxynucleotidyl transferase dUTP nick end labeling (TUNEL) positive kidney (center left), H&E stained kidney with epithelial necrosis, and tubular proteinosis and grade 4 multifocal bladder (center right) and grade 4 diffuse bladder (right). Mice were sacrificed between day 100 and 120 after treatment initiation.

FIG. 14 shows structural properties of candidate SADA domains. The sequence refers to the specific amino acids used, counting from the N-terminal amino acid. PDB ID refers to a referenced crystal structure. The molecular size of monomer displays the theoretical molecular weight for each SADA domain. The surface areas and the number of hydrogen bonds were calculated using Discovery Studio.

FIG. 15 shows the biochemical properties of candidate SADA-BsAbs (SEQ ID NO: 27, SEQ ID NO: 28) of the present technology. Total monomer size was calculated assuming 25 kDa for each scFv. Yield was calculated from at least 2 transfections using expi293 cells. Purity was determined by SEC-HPLC. High and low molecular weight impurities were defined as peaks before or after the main peak, respectively. Stability was determined by incubation at 37° C. with weekly quantitation by SEC-HPLC.

FIG. 16 shows the summary of GD2 binding kinetics of the SADA-BsAbs disclosed herein as determined by SPR. Values were calculated using a two-state reaction model. Chi² values show the error between the raw and fitted data (RU). Fold-change was calculated by dividing the K_{D} of the IgG-scFv-BsAb by the K_{D} of either P53-SADA-BsAb (SEQ ID NO: 27) or P63-SADA-BsAb (SEQ ID NO: 28).

FIG. 17 shows a summary of the pharmacokinetic properties of P53-SADA-BsAb (SEQ ID NO: 27). NSG mice (n=10) were serially bleed from 0.5 to 168 hours after intravenous BsAb administration. Pharmacokinetic analysis was carried out by non-compartmental analysis of the serum concentration-time data using WinNonlin software program (Pharsight Corp.).

FIG. 18 shows SADA PRIT dosimetry estimates calculated from mouse biodistribution studies, and their corresponding tumor-to-non-tumor ratios. Tumor bearing mice (n=3-5 per time point) were dosed with each BsAb (SEQ ID NO: 27 and SEQ ID NO: 28) (1.25 nmol) and DOTA[¹⁷⁷Lu] (18.5 MBq), 48 hours apart. Mice were sacrificed either 2, 24, 48, or 120 hours after payload delivery. IgG-scFv-BsAb treated mice received 25 μg of clearing agent 4 hours prior to the administration of DOTA[¹⁷⁷Lu].

FIG. 19 shows a summary of tissue biodistribution of DOTA[⁸⁶Y] after PET/CT scan. Tumor bearing mice were dosed with each BsAb (SEQ ID NO: 27) (1.25 nmol) and DOTA[⁸⁶Y] (3.7 MBq), 48 hours apart, and sacrificed immediately after imaging. Values are normalized to percentage of injected dose per gram of tissue (% ID/g). 2-step IgG-scFv-BsAb treated mice did not receive clearing agent (CA). 3-step IgG-scFv-BsAb treated mice received 25 mg of CA.

FIG. 20 shows a summary of serum chemistry, complete blood counts, and histopathology in nude mice treated with the indicated BsAb (SEQ ID NO: 27 and SEQ ID NO: 28)/DOTA[¹⁷⁷Lu] payload regimen. Interpretation was performed by board-certified veterinary pathologists. Normal was defined as being not significantly different from untreated age-matched littermate control mice, or within known normal ranges for this strain of mice at the same age. Histopathologic abnormalities were determined by microscopic analysis of H&E slides. Mice were submitted for assessment 111, 155 and 230 days after treatment was initiated.

FIG. 21 shows a summary of serum chemistry, complete blood counts, and histopathology in DKO mice treated with the indicated BsAb (SEQ ID NO: 27)/DOTA[¹⁷⁷Lu]payload regimen. Interpretation was performed by board-certified veterinary pathologists. Normal was defined as being not significantly different from untreated age-matched littermate control mice, or within known normal ranges for this strain of mice at the same age. Histopathologic abnormalities were determined by microscopic analysis of H&E slides. Mice were submitted for assessment 120 days after treatment was initiated.

FIG. 22A shows a summary of serum chemistry, complete blood counts, and histopathology in DKO mice treated with the indicated BsAb (SEQ ID NO: 27)/Proteus[²²⁵Ac] payload regimen. Interpretation was performed by board-certified veterinary pathologists. Normal was defined as being not significantly different from untreated age-matched littermate control mice, or within known normal ranges for this strain of mice at the same age. Histopathologic abnormalities were determined by microscopic analysis of H&E slides. Mice were submitted for assessment 80-120 days after treatment was initiated. CC-3: Cleaved caspase-3 immunohistochemistry.

FIG. 22B shows shows a summary of serum chemistry, complete blood counts, and histopathology in DKO mice treated with the indicated BsAb (SEQ ID NO: 27)/Proteus[²²⁵Ac] payload regimen. Interpretation was performed by board-certified veterinary pathologists. Normal was defined as being not significantly different from untreated age-matched littermate control mice, or within known normal ranges for this strain of mice at this age. Histopathologic abnormalities were determined by microscopic analysis of H&E slides. Mice were submitted for assessment 163, 210 and 309 days after treatment began. MF: Multifocal. Grade 1: Minimal; 2: Mild; 3: Moderate

FIG. 23A shows mean tumor responses in DOTA [¹⁷⁷Lu] small-cell lung cancer patient-derived xenograft (PDX) treatment model. Each dose of BsAb (SEQ ID NO: 27) (1.25 nmol, triangle) was followed by a dose of DOTA[177Lu] (37 kBq, 700 pmol, star) 48 hours later. Each line represents one treatment group (n=4-5). The dotted black line represents no measurable tumor, and the asterisk represents the tumor implantation. Tumor averages were calculated until at least one mouse had to be euthanized. Data are shown as means±standard deviation. FIG. 23B shows mean tumor responses in DOTA [²²⁵Ac] small-cell lung cancer patient-derived xenograft (PDX) treatment model. Each dose of BsAb (SEQ ID NO: 27) (1.25 nmol, triangle) was followed by a dose of DOTA[²²⁵Ac] (37 kBq, 700 pmol, star) 48 hours later. Each line represents one treatment group. The dotted black line represents no measurable tumor. Tumor averages were calculated until at least one mouse had to be euthanized. Data are shown as means±standard deviation. FIG. 23C shows progression-free survival analysis for each experimental group. Tumors were considered “progressing” when their volume reached 500 mm³.

FIGS. 24A-24B show in vivo biodistribution of SADA-BsAbs of the present technology. Tumor bearing nude mice (IMR32Luc sc, right flank) were treated with 1.25 nmol of each BsAb and 18.5 MBq (100 pmol) of DOTA ¹⁷⁷Lu 48 hours later. BC151 (IgG-scFv-BsAb) were treated with a clearing agent 4 hours prior to DOTALu⁷⁷. Mice were sacrificed 24 hrs after administration of Lu¹⁷⁷ and organs were collected and read on a gamma counter (Perkin Elmer). Counts were decay corrected and normalized to the injected dose (18.5 MBq and the weight of the organs). TC101=SEQ ID NO: 22, TC134=SEQ ID NO: 27, TC135=SEQ ID NO: 28, and TC135-H=SEQ ID NO: 38. Kidney uptake was not impacted by the presence or absence of a 6×HIS tag (SEQ ID NO: 41).

FIGS. 25A-25B show in vivo biodistribution of SADA-BsAbs of the present technology and their corresponding tumor-to-non-tumor ratios based on the results described in FIGS. 24A-24B (the values are normalized to the tumor uptake (tumor to blood, tumor to liver, tumor to kidney). TC101=SEQ ID NO: 22, TC134=SEQ ID NO: 27, TC135=SEQ ID NO: 28, and TC135-H=SEQ ID NO: 38. FIG. 25B represents tabulated data from FIGS. 24A-24B. Kidney uptake is not impacted by the presence or absence of a 6×HIS tag (SEQ ID NO: 41).

FIG. 26 shows the concentration of P53-SADA-BsAb (SEQ ID NO: 27) in blood at 24 hours and at 48 hours (n=5). NSG mice (n=10) were serially bleed from 0.5 to 168 hours after intravenous BsAb administration. Pharmacokinetic analysis was carried out by non-compartmental analysis of the serum concentration-time data using WinNonlin software program (Pharsight Corp.).

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001)Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

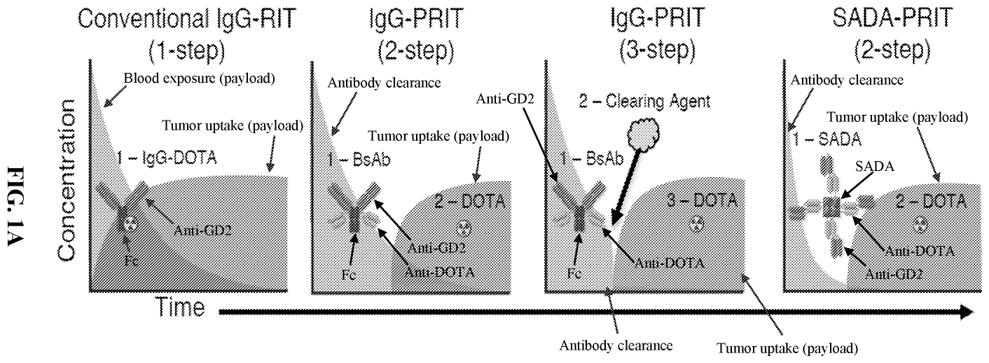

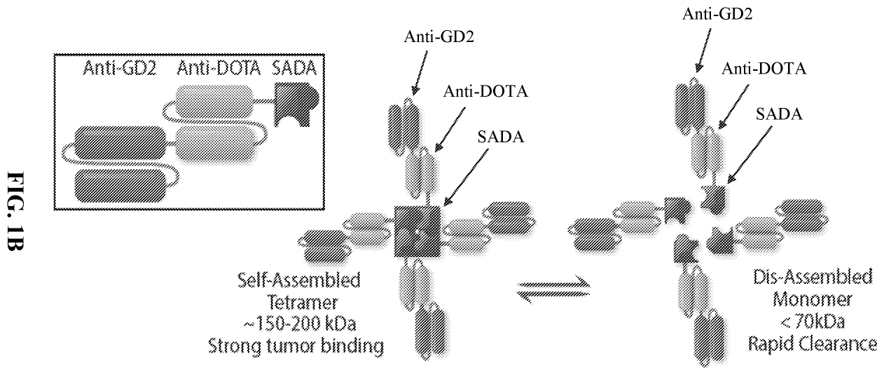

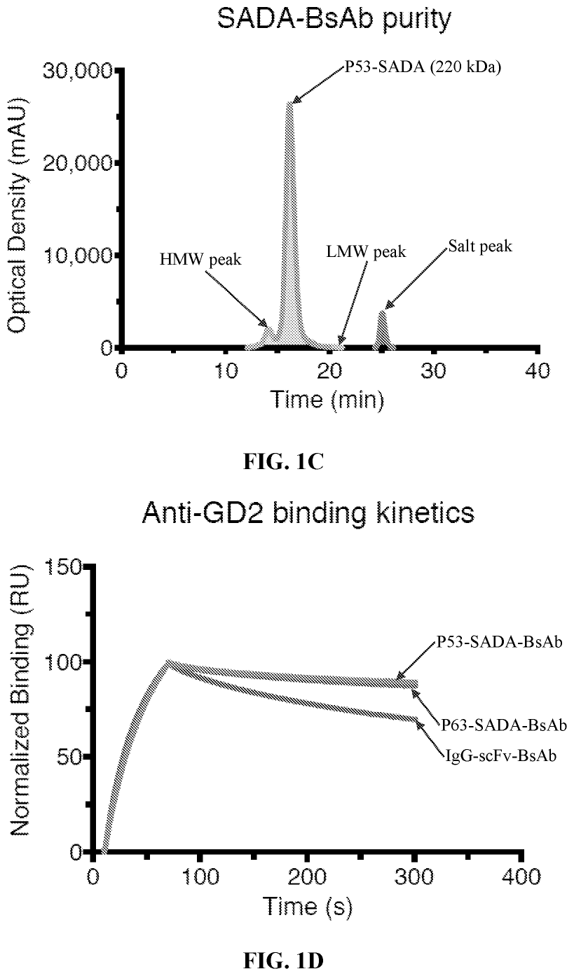

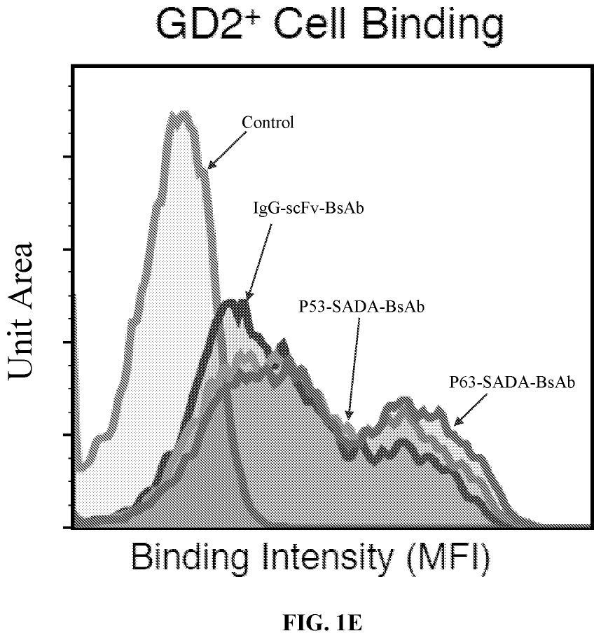

Multi-step targeting strategies are utilized to overcome TI limitations by delivering tumor targeting agents (e.g. anti-tumor IgG) separately from the cytotoxic payloads (e.g. chelated radioisotopes). As an example, conventional 2-step pretargeted radioimmunotherapy (PRIT) administers engineered bispecific antibodies (BsAb) or chemically modified monoclonal antibodies first (step 1, t_{1/2}˜days), followed hours or days later with the delivery of small radioactive payloads (step 2, t_{1/2}˜minutes) that seek out the tumor-bound antibodies (FIG. 1A). While this strategy does reduce toxicities in some tissues, the residual circulating antibody in the blood is enough to prevent any substantial improvement in therapeutic index or efficacy. One solutions is 3-step PRIT, where after the administration of tumor targeting IgG (step 1), a clearing agent step (step 2) is introduced to remove circulating antibody from the blood before the delivery of the cytotoxic payload (step 3). While inclusion of a clearing agent may improve TIs, the optimal clearing agent dose will vary depending on tumor size and antigen density, substantially complicating clinical translation. While a high dose of clearing agent should maximize removal of IgG, it could also interfere with payload uptake at the tumor. In contrast, an insufficient dose of clearing agent would leave considerable IgG in the blood, capturing the injected payload, circulating it and ultimately harming the bone marrow and other normal tissues. Thus, the ideal targeting strategy requires a tumor-targeting platform that can consistently clear itself from the blood before payload delivery without the need for optimization of additional or exogenous reagents.

The present disclosure provides a novel platform for the multi-step delivery of cytotoxic payloads to tumors using specially designed Self-Assembling and DisAssembling (SADA) domains (FIG. 1B). When fused to BsAb, the resulting SADA-BsAb self-assemble into stable tetrameric complexes (220 kDa) that bind tumors with high avidity but could also disassemble into small dimers (110 kDa) or monomers (55 kDa) after a period of circulation in the blood (hours). Importantly, while the tetrameric complexes exceed the molecular weight (MW) cut off for renal filtration, the small monomers fall below the threshold and are able to rapidly and completely clear from the blood.

While many protein therapies benefit from long terminal half-lives, the delivery of highly cytotoxic payloads using such proteins inevitably harms sensitive tissues such as bone marrow. To date, all 8 FDA approved antibody-drug conjugates, and both FDA approved radiolabeled protein therapies have demonstrated some myelotoxicity during clinical development, using substantially lower doses of payload than were achieved with the 2-step SADA-PRIT methods disclosed herein. The present disclosure demonstrates that the SADA-BsAbs of the present technology in combination with radioactive payloads carrying alpha (²²⁵Ac 1.48 MBq/kg) or beta (¹⁷⁷Lu 6,660 MBq/kg) radioisotopes ablate established solid tumors in multiple mouse models without the need for any clearing agent. Instead, the SADA-platform utilized the narrow window in blood retention between long-lived large size proteins and small peptides, temporarily maintaining a plasma half-life for just enough time to effectively reach the tumor, followed by rapid and complete clearance from the blood. Additionally, this fast clearance rendered SADA-BsAb substantially less immunogenic compared to more conventional IgG-based platforms, a crucial advantage in therapeutic strategies that necessitate multiple treatment cycles.

The methods disclosed herein eliminated all clinical or histologic toxicities to the kidneys, liver, bone marrow, spleen, or brain while delivering enormous doses of cytotoxic payloads. These results are critical and clinically relevant given the sensitivity of these organs to radiation-related toxicities in conventional RIT. See e.g., Bodei et al., European Journal of Nuclear Medicine and Molecular Imaging 42, 5-19 (2015); Forster et al., Journal of Nuclear Medicine 47, 140-149 (2006); Gupta et al., Cancer Biotherapy and Radiopharmaceuticals 27, 593-599 (2012); Heskamp, et al., Journal of Nuclear Medicine 58, 926-933 (2017); Muselaers et al., Journal of Nuclear Medicine 57, 34-34 (2016); Poty et al., Clinical Cancer Research 25, 868-880 (2019); Vallabhajosula et al., Journal of Nuclear Medicine 46, 850-858 (2005). In particular, the myelotoxicity-free dose levels achieved in the present disclosure (up to 6,600 MBq/kg) are exponentially higher than those currently used in the clinic (typically <150 MBq/kg), demonstrating the safety margin that SADA-BsAb provided to radiosensitive tissues.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 103 M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a p-sheet conformation and the CDRs form loops which connect, and in some cases form part of, the 0-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target antigen (e.g., GD2) will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multi-specific antibodies, bispecific antibodies, etc.,) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multi-specific antibodies formed from antibody fragments.

“Bispecific antibody” or “BsAb”, as used herein, refers to an immunoglobulin-related composition that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may GD2. An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

“Binding domain”, as used herein, refers to a moiety or entity that specifically binds to a target moiety or entity. Typically, the interaction between a binding domain and its target is non-covalent. In some embodiments, a binding domain may be or comprise a moiety or entity of any chemical class including, for example, a carbohydrate, a lipid, a nucleic acid, a metal, a polypeptide, a small molecule. In some embodiments, a binding domain may be or comprise a polypeptide (or complex thereof), a target-binding portion of an immunoglobulin-related composition, a cytokine, a ligand (e.g., a receptor ligand), a receptor, a toxin, etc. In certain embodiments, a binding domain may be or comprise an aptamer. In other embodiments, a binding domain may be or comprise a peptide nucleic acid (PNA).

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, a “clearing agent” is an agent that binds to excess bifunctional antibody that is present in the blood compartment of a subject to facilitate rapid clearance via kidneys. The use of the clearing agent prior to hapten administration facilitates better tumor-to-background ratios in PRIT systems. Examples of clearing agents include 500 kD-dextran-DOTA-Bn(Y) (Orcutt et al., Mol Cancer Ther. 11(6): 1365-1372 (2012)), 500 kD aminodextran-DOTA conjugate, antibodies against the pretargeting antibody, etc.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

“Dosage form” and “unit dosage form”, as used herein, the term “dosage form” refers to physically discrete unit of a therapeutic agent for a subject (e.g., a human patient) to be treated. Each unit contains a predetermined quantity of active material calculated or demonstrated to produce a desired therapeutic effect when administered to a relevant population according to an appropriate dosing regimen. For example, in some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). It will be understood, however, that the total dosage administered to any particular patient will be selected by a medical professional (e.g., a medical doctor) within the scope of sound medical judgment.

“Dosing regimen” (or “therapeutic regimen”), as used herein is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in certain embodiments, a dosing regimen comprises a plurality of doses and at least two different time periods separating individual doses. In some embodiments, the therapeutic agent is administered continuously (e.g., by infusion) over a predetermined period. In other embodiments, a therapeutic agent is administered once a day (QD) or twice a day (BID). In some embodiments, a dosing regimen comprises a plurality of doses each of which are separated from one another by a time period of the same length; in other embodiments, a dosing regimen comprises a plurality of doses and at least two different time periods separating individual doses. In some embodiments, all doses within a dosing regimen are of the same unit dose amount. In certain embodiments, different doses within a dosing regimen are of different amounts. In some embodiments, a dosing regimen comprises a first dose in a first dose amount, followed by one or more additional doses in a second dose amount different from the first dose amount. In other embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, M.D. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding a SADA-BsAb described herein or amino acid sequence of a SADA-BsAb described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the terms “individual”, “patient”, or “subject” can be an individual organism, a vertebrate, a mammal, or a human. In some embodiments, the individual, patient or subject is a human.

“K_{D}”, as used herein, refers to the dissociation constant of a binding domain (e.g., a SADA domain, an antibody or binding component thereof) from a complex with its partner (e.g., a corresponding SADA domain or an epitope to which the antibody or binding component thereof binds).

As used herein, “k_{off}” refers to the off rate constant for dissociation of a binding agent (e.g., a SADA domain, an antibody or binding component thereof) from a complex with its partner (e.g., a corresponding SADA domain or an epitope to which the antibody or binding component thereof binds).

As used herein, “kₒₙ” refers to the on rate constant for association of a binding agent (e.g., a SADA domain, an antibody or binding component thereof) with its partner (e.g., a corresponding SADA domain or an epitope to which the antibody or binding component thereof binds).

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, “linker” typically refers to a portion of a molecule or entity that connects two or more different regions of interest (e.g., particular structural and/or functional domains or moieties of interest). The linker may lack a defined or rigid structure and/or may not materially alter the relevant function of the domain(s) or moiety(ies) within the two or more different regions of interest. In some embodiments, the linker is or comprises a polypeptide and may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids long. In certain embodiments, a polypeptide linker may have an amino acid sequence that is or comprises GGGGS GGGGS GGGGS (i.e., [G₄S]₃) (SEQ ID NO: 19), GGGGS GGGGS GGGGS GGGGS (i.e., [G₄S]₄) (SEQ ID NO: 20), or GGGGS GGGGS GGGGS GGGGS GGGGS GGGGS (i.e., [G₄S]₆) (SEQ ID NO: 21).

As used herein, a “multimer” refers to a complex of monomeric units and may include trimers, and multimers of four monomers (tetramers), or of more than four monomers (pentamers, hexamers, septamers, octamers, nonamers, decamers, etc.). A domain that promotes association of monomeric units to form multimeric complexes is referred to as a “multimerization domain.”

“Payload”, as used herein, refers to a moiety or entity that is delivered to a site of interest (e.g., to a cell, tissue, tumor, or organism) by association with another entity. In some embodiments, a payload is or comprises a detection agent or a therapeutic agent. Those of ordinary skill in the art will appreciate that a payload entity may be of any chemical class. For example, in some embodiments, a payload entity may be or comprise a carbohydrate, an isotope, a lipid, a nucleic acid, a metal, a nanoparticle (e.g., a ceramic or polymer nanoparticle), polypeptide, a small molecule, etc. To give but a few examples, in some embodiments, a therapeutic agent payload may be or comprise a toxin (e.g., a toxic peptide, small molecule, or isotope [e.g., radioisotope]); in some embodiments, a detection agent payload may be or comprise a fluorescent entity or agent, a radioactive entity or agent, an agent or entity detectable by binding (e.g., a tag, a hapten, a ligand, etc.), a catalytic agent, etc.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

The term “radioactive isotope” as used herein has its art-understood meaning referring to an isotope that undergoes radioactive decay. In some embodiments, a radioactive isotope may be or comprise one or more of actinium-225, astatine-211, bismuth-212, carbon-14, chromium-51, chlorine-36, cobalt-57, cobalt-58, copper-67, Europium-152, gallium-67, hydrogen-3, iodine-123, iodine-124, iodine-125, iodine-131, indium-111, iron-59, lead-212, lutetium-177, phosphorus-32, radium-223, radium-224, rhenium-186, rhenium-188, selenium-75, sulphur-35, technicium-99m, thorium-227, yttrium-90, and zirconium-89.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹²M. The term “specifically binds” may also refer to binding where a molecule (e.g., an antibody or antigen binding fragment thereof) binds to a particular antigen (e.g., GD2), or an epitope on a particular antigen, without substantially binding to any other antigen, or antigen epitope.

As used herein, the term “simultaneous” therapeutic use refers to the administration of at least two active ingredients by the same route and at the same time or at substantially the same time.

“Surface plasmon resonance”, as used herein, refers to an optical phenomenon that allows for the analysis of specific binding interactions in real-time, for example through detection of alterations in protein concentrations within a biosensor matrix, such as by using a BIAcore system (Pharmacia Biosensor AB, Uppsala, Sweden and Piscataway, N.J.). For further descriptions, see Jonsson, U., et al. (1993) Ann. Biol. Clin. 51: 19-26; Jonsson, U., et al, (1991) Biotechniques 11:620-627; Johnsson, B., et al, (1995) J. Mol. Recognit. 8: 125-131; and Johnnson, B., et al, (1991) Anal Biochem. 198:268-277.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-GD2 SADA conjugates described herein are contemplated. For example, it may be desirable to improve the binding affinity and/or other biological properties of the anti-GD2 SADA conjugate. Amino acid sequence variants of an anti-GD2 SADA conjugate are prepared by introducing appropriate nucleotide changes into the anti-GD2 SADA conjugate nucleic acid, or by peptide synthesis. Such modifications include, for example, deletions from, and/or insertions into and/or substitutions of, residues within the amino acid sequences of the anti-GD2 SADA conjugate. Any combination of deletion, insertion, and substitution is made to obtain the anti-GD2 SADA conjugate of interest, as long as the obtained anti-GD2 SADA conjugate possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

TABLE 1
Amino Acid Substitutions

| Original | Exemplary | Conservative |
|---|---|---|
| Residue | Substitutions | Substitutions |
| Ala (A) | val; leu; ile | val |
| Arg (R) | lys; gln; asn | lys |
| Asn (N) | gln; his; asp, lys; | gln |
| | arg | |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; | leu |
| | phe; norleucine | |
| Leu (L) | norleucine; ile; | ile |
| | val; met; ala; phe | |
| Lys (K) | arg; gln; asn | arg |
| Met (M) | leu; phe; ile | leu |
| Phe (F) | leu; val; ile; ala; | tyr |
| | tyr | |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

### Anti-GD2 SADA Conjugate Compositions of the Present Technology

The anti-GD2 SADA conjugates (e.g., anti-DOTA bispecific antigen binding fragments) of the present technology comprise a self-assembly disassembly (SADA) polypeptide of P53 or P63, fused to a GD2-specific antigen binding domain, and a DOTA-specific antigen binding domain. In some embodiments, such conjugates are characterized in that they multimerize to form a complex of a desired size under relevant conditions (e.g., in a solution in which the conjugate is present above a threshold concentration or pH and/or when present at a target site characterized by a relevant level or density of receptors for the payload), and disassemble to a smaller form under other conditions (e.g., absent the relevant environmental multimerization trigger).

A SADA domain is composed of multimerization domains which are each composed of helical bundles that associate in a parallel or anti-parallel orientation. Examples of SADA domain containing human polypeptides include p53, p63, p73, heterogeneous nuclear Ribonucleoprotein (hnRNPC) C, or N-terminal domain of Synaptosomal-associated protein 23 (SNAP-23), Cyclin-D-related protein (CBFA2T1), or variants or fragments thereof. See FIG. 14. Exemplary amino acid sequences of human p53 tetramerization domain and p63 tetramerization domain are provided below:

Human p53 tetramerization domain amino acid
sequence (321-359)
(SEQ ID NO: 36)
KPLDGEYFTLQIRGRERFEMFRELNEALELKDAQAGKEP
Human p63 tetramerization domain amino acid
sequence (396-450)
(SEQ ID NO: 37)
RSPDDELLYLPVRGRETYEMLLKIKESLELMQYLPQHTIETYRQQQQQQH
QHLLQKQ

Each of the GD2-specific antigen binding domain and the DOTA-specific antigen binding domain of the anti-GD2 SADA conjugates disclosed herein may comprise a heavy chain variable domain (V_{H}) sequence and a light chain variable domain (V_{L}) sequence. Exemplary V_{H} and V_{L} amino acid sequences of the GD2-specific antigen binding domain of the anti-GD2 SADA conjugates are provided below:

hu3F8 V_{H}
(SEQ ID NO: 1)
QVQLVESGPGVVQPGRSLRISCAVSGFSVTNYGVHWVRQPPGKCLEWLGV
IWAGGITNYNSAFMSRLTISKDNSKNTVYLQMNSLRAEDTAMYYCASRGG
HYGYALDYWGQGTLVTVSS
hu3F8 V_{L}
(SEQ ID NO: 5)
EIVMTQTPATLSVSAGERVTITCKASQSVSNDVTWYQQKPGQAPRLLIYS
ASNRYSGVPARFSGSGYGTEFTFTISSVQSEDFAVYFCQQDYSSFGCGTK
LEIKR

The V_{H} CDR₁, V_{H} CDR₂ and V_{H} CDR₃ sequences of SEQ ID NO: 1 are NYGVH (SEQ ID NO: 2), VIWAGGITNYNSAFMS (SEQ ID NO: 3), and RGGHYGYALDY (SEQ ID NO: 4), respectively, and are underlined in order of appearance. The V_{L} CDR₁, V_{L} CDR₂ and V_{L} CDR₃ sequences of SEQ ID NO: 5 are KASQSVSNDVT (SEQ ID NO: 6), SASNRYS (SEQ ID NO: 7), and QQDYSS (SEQ ID NO: 8), respectively, and are underlined in order of appearance.

Exemplary V_{H} and V_{L} amino acid sequences of the DOTA-specific antigen binding domain of the anti-GD2 SADA conjugates are provided below:

huC825 V_{H}
(SEQ ID NO: 9)
HVQLVESGGGLVQPGGSLRLSCAASGFSLTDYGVHWVRQAPGKGLEWLGV
IWSGGGTAYNTALISRFTISRDNSKNTLYLQMNSLRAEDTAVYYCARRGS
YPYNYFDAWGCGTLVTVSS
huC825 V_{L}
(SEQ ID NO: 13)
QAVVTQEPSLTVSPGGTVTLTCGSSTGAVTASNYANWVQQKPGQCPRGLI
GGHNNRPPGVPARFSGSLLGGKAALTLLGAQPEDEAEYYCALWYSDHWVI
GGGTKLTVLG
C825 V_{H}
(SEQ ID NO: 17)
HVKLQESGPGLVQPSQSLSLTCTVSGFSLTDYGVHWVRQSPGKGLEWLGV
IWSGGGTAYNTALISRLNIYRDNSKNQVFLEMNSLQAEDTAMYYCARRGS
YPYNYFDAWGCGTTVTVSS
C825 V_{L}
(SEQ ID NO: 18)
QAVVIQESALTTPPGETVTLTCGSSTGAVTASNYANWVQEKPDHCFTGLI
GGHNNRPPGVPARFSGSLIGDKAALTIAGTQTEDEAIYFCALWYSDHWVI
GGGTRLTVLG

The V_{H} CDR₁, V_{H} CDR₂ and V_{H} CDR₃ sequences of SEQ ID NOs: 9 and 17 are DYGVH (SEQ ID NO: 10), VIWSGGGTAYNTALIS (SEQ ID NO: 11), RGSYPYNYFDA (SEQ ID NO: 12), respectively, and are underlined in order of appearance. The V_{L} CDR₁, V_{L} CDR₂ and V_{L} CDR₃ sequences of SEQ ID NOs: 13 and 18 are GSSTGAVTASNYAN (SEQ ID NO: 14), GHNNRPP (SEQ ID NO: 15), and ALWYSDHWV (SEQ ID NO: 16), respectively, and are underlined in order of appearance.

In some embodiments, the GD2-specific antigen binding domain of the anti-GD2 SADA conjugates comprise a heavy chain variable domain (V_{H}) sequence and a light chain variable domain (V_{L}) sequence of SEQ ID NO: 1 and SEQ ID NO: 5, respectively. Additionally or alternatively, in some embodiments, the DOTA-specific antigen binding domain of the anti-GD2 SADA conjugates comprise a heavy chain variable domain (V_{H}) sequence of SEQ ID NO: 9 or SEQ ID NO: 17, and a light chain variable domain (V_{L}) sequence of SEQ ID NO: 13 or SEQ ID NO: 18. In any and all embodiments of the anti-GD2 SADA conjugates of the present technology, the SADA polypeptide is or comprises a tetramerization domain of p53, or p63. In some embodiments, the SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 36, and 37. In some embodiments, the SADA polypeptide is or comprises a sequence that is at least 75%, 80%, 85%, 90%, 91%, 92%, 93%, 94%, 95%, 96%, 97%, 98%, or 99% identical to a sequence as set forth in any one of SEQ ID NOs: 36, and 37, and wherein the underlined amino acid residues in these sequences above are conserved.

Additionally or alternatively, in certain embodiments of the anti-GD2 SADA conjugates of the present technology, the SADA polypeptide is covalently linked to the GD2-specific antigen binding domain, or the DOTA-specific antigen binding domain via a linker. Any suitable linker known in the art can be used. In some embodiments, the SADA polypeptide is linked to the GD2-specific antigen binding domain, or the DOTA-specific antigen binding domain via a polypeptide linker. In certain embodiments, the polypeptide linker is a Gly-Ser linker. In further embodiments, a polypeptide linker is or comprises a sequence of (GGGGS)n (SEQ ID NO: 42), where n represents the number of repeating GGGGS units and is 1, 2, 3, 4, 5, 6, 7, 8, 9, 10, 11, 12, 13, 14, 15, 16, 17, 18, 19, 20, 25, 30 or more. In other embodiments of the anti-GD2 SADA conjugates, the SADA polypeptide is directly fused to the GD2-specific antigen binding domain, or the DOTA-specific antigen binding domain.

In any of the preceding embodiments of the anti-GD2 SADA conjugates disclosed herein, the V_{H} domain sequence and the V_{L} domain sequence in the GD2-specific antigen binding may be linked via an intra-peptide linker. In certain embodiments, the intra-peptide linker is a Gly-Ser linker or comprises a sequence of (GGGGS)n (SEQ ID NO: 42), where n represents the number of repeating GGGGS units and is 1, 2, 3, 4, 5, 6, 7, 8, 9, 10, 11, 12, 13, 14, 15, 16, 17, 18, 19, 20, 25, 30 or more. Additionally or alternatively, in some embodiments, the sequence of the intra-peptide linker between the V_{H} domain sequence and the V_{L} domain sequence in the GD2-specific antigen binding domain is any one of SEQ ID NOs: 19-21.

In any and all embodiments of the anti-GD2 SADA conjugates disclosed herein, the V_{H} domain sequence and the V_{L} domain sequence in the DOTA-specific antigen binding may be linked via an intra-peptide linker. In certain embodiments, the intra-peptide linker is a Gly-Ser linker or comprises a sequence of (GGGGS)n (SEQ ID NO: 42), where n represents the number of repeating GGGGS units and is 1, 2, 3, 4, 5, 6, 7, 8, 9, 10, 11, 12, 13, 14, 15, 16, 17, 18, 19, 20, 25, 30 or more. Additionally or alternatively, in some embodiments, the sequence of the intra-peptide linker between the V_{H} domain sequence and the V_{L} domain sequence in the DOTA-specific antigen binding domain is any one of SEQ ID NOs: 19-21.

In any and all embodiments of the anti-GD2 SADA conjugates of the present technology, the GD2-specific antigen binding domain and the DOTA-specific antigen binding domain may be linked via an intra-peptide linker. In certain embodiments, the intra-peptide linker is a Gly-Ser linker or comprises a sequence of (GGGGS)n (SEQ ID NO: 42), where n represents the number of repeating GGGGS units and is 1, 2, 3, 4, 5, 6, 7, 8, 9, 10, 11, 12, 13, 14, 15, 16, 17, 18, 19, 20, 25, 30 or more. Additionally or alternatively, in some embodiments, the sequence of the intra-peptide linker between the GD2-specific antigen binding domain and the DOTA-specific antigen binding domain is any one of SEQ ID NOs: 19-21.

In certain embodiments, the anti-GD2 SADA conjugate of the present technology comprises a first polypeptide chain, wherein the first polypeptide chain comprises in the N-terminal to C-terminal direction: (i) the V_{L} sequence of SEQ ID NO: 5; (ii) a flexible peptide linker comprising the amino acid sequence of any one of SEQ ID NOs: 19-21; (iii) the V_{H} sequence of SEQ ID NO: 1; (iv) a flexible peptide linker comprising the amino acid sequence of any one of SEQ ID NOs: 19-21; (v) the V_{H} sequence of SEQ ID NO: 9 or SEQ ID NO: 17; (vi) a flexible peptide linker comprising the amino acid sequence of any one of SEQ ID NOs: 19-21; (vii) the V_{L} sequence of SEQ ID NO: 13 or SEQ ID NO: 18; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In some embodiments, the anti-GD2 SADA conjugate of the present technology comprises a first polypeptide chain, wherein the first polypeptide chain comprises in the N-terminal to C-terminal direction: (i) the V_{L} sequence of SEQ ID NO: 5; (ii) a flexible peptide linker comprising the amino acid sequence of any one of SEQ ID NOs: 19-21; (iii) the V_{H} sequence of SEQ ID NO: 1; (iv) a flexible peptide linker comprising the amino acid sequence of any one of SEQ ID NOs: 19-21; (v) the V_{L} sequence of SEQ ID NO: 13 or SEQ ID NO: 18; (vi) a flexible peptide linker comprising the amino acid sequence of any one of SEQ ID NOs: 19-21; (vii) the V_{H} sequence of SEQ ID NO: 9 or SEQ ID NO: 17; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In other embodiments, the anti-GD2 SADA conjugate of the present technology comprises a first polypeptide chain, wherein the first polypeptide chain comprises in the N-terminal to C-terminal direction: (i) the V_{H} sequence of SEQ ID NO: 1; (ii) a flexible peptide linker comprising the amino acid sequence of any one of SEQ ID NOs: 19-21; (iii) the V_{L} sequence of SEQ ID NO: 5; (iv) a flexible peptide linker comprising the amino acid sequence of any one of SEQ ID NOs: 19-21; (v) the V_{H} sequence of SEQ ID NO: 9 or SEQ ID NO: 17; (vi) a flexible peptide linker comprising the amino acid sequence of any one of SEQ ID NOs: 19-21; (vii) the V_{L} sequence of SEQ ID NO: 13 or SEQ ID NO: 18; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

In some embodiments, the anti-GD2 SADA conjugate of the present technology comprises a first polypeptide chain, wherein the first polypeptide chain comprises in the N-terminal to C-terminal direction: (i) the V_{H} sequence of SEQ ID NO: 1; (ii) a flexible peptide linker comprising the amino acid sequence of any one of SEQ ID NOs: 19-21; (iii) the V_{L} sequence of SEQ ID NO: 5; (iv) a flexible peptide linker comprising the amino acid sequence of any one of SEQ ID NOs: 19-21; (v) the V_{L} sequence of SEQ ID NO: 13 or SEQ ID NO: 18; (vi) a flexible peptide linker comprising the amino acid sequence of any one of SEQ ID NOs: 19-21; (vii) the V_{H} sequence of SEQ ID NO: 9 or SEQ ID NO: 17; (viii) a flexible peptide linker sequence comprising the amino acid sequence TPLGDTTHT (SEQ ID NO: 40); and (ix) a self-assembly disassembly (SADA) polypeptide sequence of SEQ ID NO: 36 or SEQ ID NO: 37.

Exemplary anti-GD2 SADA conjugates (e.g., anti-DOTA bispecific antigen binding fragments) of the present technology are provided below:

Anti-GD2 × anti-DOTA P53 SADA (noHIS) polypeptide (hu3F8-scFv, GS linker,
huC825-scFv, (IgG3 spacer), huP53-tet)
(SEQ ID NO: 22)
EIVMTQTPATLSVSAGERVTITCKASQSVSNDVTWYQQKPGQAPRLLIYSASNRYSGVP
ARFSGSGYGTEFTFTISSVOSEDFAVYFCQQDYSSFGCGTKLEIKRGGGGSGGGGSGGGG
SQVQLVESGPGVVQPGRSLRISCAVSGFSVTNYGVHWVRQPPGKCLEWLGVIWAGGIT
NYNSAFMSRLTISKDNSKNTVYLQMNSLRAEDTAMYYCASRGGHYGYALDYWGQGT
LVTVSSGGGGSGGGGSGGGGSGGGGSHVQLVESGGGLVQPGGSLRLSCAASGFSLTDYGV
HWVRQAPGKGLEWLGVIWSGGGTAYNTALISRFTISRDNSKNTLYLQMNSLRAEDTAVYYCAR
RGSYPYNYFDAWGCGTLVTVSSGGGGSGGGGSGGGGSQAVVTQEPSLTVSPGGTVTLTCGSS
TGAVTASNYANWVQQKPGQCPRGLIGGHNNRPPGVPARFSGSLLGGKAALTLLGAQPEDEA
EYYCALWYSDHWVIGGGTKLTVLG(TPLGDTTHT)SGKPLDGEYFTLQIRGRERFEMFRE
LNEALELKDAQAGKEPGGSGGA
Anti-GD2 × anti-DOTA P53 SADA (LS) polypeptide (hu3F8-scFv, GS linker, huC825-scFv,
(IgG3 spacer), huP53-tet)
(SEQ ID NO: 23)
EIVMTQTPATLSVSAGERVTITCKASQSVSNDVTWYQQKPGQAPRLLIYSASNRYSGVP
ARFSGSGYGTEFTFTISSVOSEDFAVYFCOQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGQGTLVTVSSGGGGSGGGGSGGGGSGGGGSHVQLVESGGGLVQPGGSL
RLSCAASGFSLTDYGVHWVRQAPGKGLEWLGVIWSGGGTAYNTALISRFTISRDNSKNTLYLQ
MNSLRAEDTAVYYCARRGSYPYNYFDAWGCGTLFTVSSGGGGSGGGGSGGGGSQAVVTQEP
SLTVSPGGTVTLTCGSSTGAVTASNYANWVQQKPGQCPRGLIGGHNNRPPGVPARFSGSLLG
GKAALTLLGAQPEDEAEYYCALWYSDHWVIGGGTKLTVLG(TPLGDTTHT)SGKPLDGEYF
TLQIRGRERFEMFRELNEALELKDAQAGKEPGGSGGAPHHHHHH
Anti-GD2 × anti-DOTA P63 SADA (LS) polypeptide (hu3F8-scFv, GS linker, huC825-scFv,
(IgG3 spacer), huP63-tet)
(SEQ ID NO: 24)
EIVMTQTPATLSVSAGERVTITCKASQSVSNDVTWYQQKPGQAPRLLIYSASNRYSGVP
ARFSGSGYGTEFTFTISSVOSEDFAVYFCOQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGQGTLVTVSSGGGGSGGGGSGGGGSGGGGSHVQLVESGGGLVQPGGSL
RLSCAASGFSLTDYGVHWVRQAPGKGLEWLGVIWSGGGTAYNTALISRFTISRDNSKNTLYLQ
MNSLRAEDTAVYYCARRGSYPYNYFDAWGCGTLVTVSSGGGGSGGGGSGGGGSQAVVTQEP
SLTVSPGGTVTLTCGSSTGAVTASNYANWVQQKPGQCPRGLIGGHNNRPPGVPARFSGSLLG
GKAALTLLGAQPEDEAEYYCALWYSDHWVIGGGTKLTVLG(TPLGDTTHT)SGRSPDDELLY
LPVRGRETYEMLLKIKESLELMQYLPQHTIETYRQQQQQQHQHLLQKQGGSGGAP
HHHHHH
Anti-GD2 × anti-DOTA P53 SADA (SS) polypeptide (hu3F8-scFv, GS linker, huC825-scFv,
(IgG3 spacer), huP53-tet)
(SEQ ID NO: 25)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SQVQLVESGPGVVQPGRSLRISCAVSGFSVTNYGVHWVRQPPGKCLEWLGVIWAGGIT
NYNSAFMSRLTISKDNSKNTVYLQMNSLRAEDTAMYYCASRGGHYGYALDYWGQGT
LVYVSSGGGGSGGGGSGGGGSGGGGSHVQLVESGGGLVQPGGSLRLSCAASGFSLTDYGV
HWVRQAPGKGLEWLGVIWSGGGTAYNTALISRFTISRDNSKNTLYLQMNSLRAEDTAVYYCAR
RGSYPYNYFDAWGCGTLVTVSSGGGGSGGGGSGGGGSQAWTQEPSLTVSPGGTVTLTCGSS
TGAVTASNYANWVQQKPGQCPRGLIGGHNNRPPGVPARFSGSLLGGKAALTLLGAQPEDEA
EYYCALWYSDHWVIGGGTKLTVLG(TPLGDTTHT)SGKPLDGEYFTLQIRGRERFEMFRE
LNEALELKDAQAGKEPGGSGGAPHHHHHH
Anti-GD2 × anti-DOTA P63 SADA (SS) polypeptide (hu3F8-scFv, GS linker, huC825-scFv,
(IgG3 spacer), huP63-tet)
(SEQ ID NO: 26)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SQVQLVESGPGVVQPGRSLRISCAVSGFSVTNYGVHWVRQPPGKCLEWLGVIWAGGIT
NYNSAFMSRLTISKDNSKNTVYLQMNSLRAEDTAMYYCASRGGHYGYALDYWGQGT
LVYVSSGGGGSGGGGSGGGGSGGGGSHVQLVESGGGLVQPGGSLRLSCAASGFSLTDYGV
HWVRQAPGKGLEWLGVIWSGGGTAYNTALISRFTISRDNSKNTLYLQMNSLRAEDTAVYYCAR
RGSYPYNYFDAWGCGTLVTVSSGGGGSGGGGSGGGGSQAWTQEPSLTVSPGGTVTLTCGSS
TGAVTASNYANWVQQKPGQCPRGLIGGHNNRPPGVPARFSGSLLGGKAALTLLGAQPEDEA
EYYCALWYSDHWVIGGGTKLTVLG(TPLGDTTHT)SGRSPDDELLYLPVRGRETYEMLLK
IKESLELMQYLPQHTIETYRQQQQQQHQHLLQKQGGSGGAPHHHHHH
Anti-GD2 X anti-DOTA P53 SADA (LL) polypeptide (hu3F8-scFv, GS linker, huC825-scFv,
(IgG3 spacer), huP53-tet)
(SEQ ID NO: 27)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGQGTLVTVSSGGGGSGGGGSGGGGSGGGGSHVQLVESGGGLVQPGGSL
RLSCAASGFSLTDYGVHWVRQAPGKGLEWLGVIWSGGGTAYNTALISRFTISRDNSKNTLYLQ
MNSLRAEDTAVYYCARRGSYPYNYFDAWGCGTLVTVSSGGGGSGGGGSGGGGSGGGGSGG
GGSGGGGSQAVVTQEPSLTVSPGGTVTLTCGSSTGAVTASNYANWVQQKPGQCPRGLIGGH
NNRPPGVPARFSGSLLGGKAALTLLGAQPEDEAEYYCALWYSDHWVIGGGTKLTVLG(TPLG
DTTHT)SGKPLDGEYFTLQIRGRERFEMFRELNEALELKDAQAGKEPGGSGGAPHH
HHHH
Anti-GD2 × anti-DOTA P63 SADA (LL) polypeptide (hu3F8-scFv, GS linker, huC825-scFv,
(IgG3 spacer), huP63-tet)
(SEQ ID NO: 28)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGOGTLVTVSSGGGGSGGGGSGGGGSGGGGSHVQLVESGGGLVQPGGSL
RLSCAASGFSLTDYGVHWVRQAPGKGLEWLGVIWSGGGTAYNTALISRFTISRDNSKNTLYLQ
MNSLRAEDTAVYYCARRGSYPYNYFDAWGCGTLVTVSSGGGGSGGGGSGGGGSGGGGSGG
GGSGGGGSQAVVTQEPSLTVSPGGTVTLTCGSSTGAVTASNYANWVQQKPGQCPRGLIGGH
NNRPPGVPARFSGSLLGGKAALTLLGAQPEDEAEYYCALWYSDHWVIGGGTKLTVLG(TPLG
DTTHT)SGRSPDDELLYLPVRGRETYEMLLKIKESLELMQYLPQHTIETYRQQQQQ
QHQHLLQKQGGSGGAPHHHHHH
Anti-GD2 × murine anti-DOTA P53 SADA (noHIS) polypeptide (hu3F8-scFv, GS linker, C825-
scFv, (IgG3 spacer), huP53-tet)
(SEQ ID NO: 29)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SQVQLVESGPGVVQPGRSLRISCAVSGFSVTNYGVHWVRQPPGKCLEWLGVIWAGGIT
NYNSAFMSRLTISKDNSKNTVYLQMNSLRAEDTAMYYCASRGGHYGYALDYWGQGT
LVTVSSGGGGSGGGGSGGGGSGGGGSHVKLQESGPGLVQPSQSLSLTCTVSGFSLTDYGV
HWVRQSPGKGLEWLGVIWSGGGTAYNTALISRLNIYRDNSKNQVFLEMNSLQAEDTAMYYCA
RRGSYPYNYFDAWGCGTTVTVSSGGGGSGGGGSGGGGSQAVVIQESALTTPPGETVTLTCGS
STGAVTASNYANWVQEKPDHCFTGLIGGHNNRPPGVPARFSGSLIGDKAALTIAGTQTEDEAI
YFCALWYSDHWVIGGGTRLTVLG(TPLGDTTHT)SGKPLDGEYFTLQIRGRERFEMFREL
NEALELKDAQAGKEPGGSGGA
Anti-GD2 × murine anti-DOTA P53 SADA (LS) polypeptide (hu3F8-scFv, GS linker, C825-
scFv, (IgG3 spacer), huP53-tet)
(SEQ ID NO: 30)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGQGTLVTVSSGGGGSGGGGSGGGGSGGGGSHVKLQESGPGLVQPSQSLS
LTCTVSGFSLTDYGVHWVRQSPGKGLEWLGVIWSGGGTAYNTALISRLNIYRDNSKNQVFLEM
NSLQAEDTAMYYCARRGSYPYNYFDAWGCGTTVTVSSGGGGSGGGGSGGGGSQAVVIQESA
LTTPPGETVTLTCGSSTGAVTASNYANWVQEKPDHCFTGLIGGHNNRPPGVPARFSGSLIGDK
AALTIAGTQTEDEAIYFCALWYSDHWVIGGGTRLTVLG(TPLGDTTHT)SGKPLDGEYFTLQI
RGRERFEMFRELNEALELKDAQAGKEPGGSGGAPHHHHHH
Anti-GD2 × murine anti-DOTA P63 SADA (LS) polypeptide (hu3F8-scFv, GS linker, C825-
scFv, (IgG3 spacer), huP63-tet)
(SEQ ID NO: 31)
EIVMTQTPATLSVSAGERVTITCKASQSVSNDVTWYQQKPGQAPRLLIYSASNRYSGVP
ARFSGSGYGTEFTFTISSVOSEDFAVYFCOQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGQGTLVTVSSGGGGSGGGGSGGGGSGGGGSHVKLQESGPGLVQPSQSLS
LTCTVSGFSLTDYGVHWVRQSPGKGLEWLGVIWSGGGTAYNTALISRLNIYRDNSKNQVFLEM
NSLQAEDTAMYYCARRGSYPYNYFDAWGCGTTFTVSSGGGGSGGGGSGGGGSQAWIQESA
LTTPPGETVTLTCGSSTGAVTASNYANWVQEKPDHCFTGLIGGHNNRPPGVPARFSGSLIGDK
AALTIAGTQTEDEAIYFCALWYSDHWVIGGGTRLTVLG(TPLGDTTHT)SGRSPDDELLYLPV
RGRETYEMLLKIKESLELMQYLPQHTIETYRQQQQQQHQHLLQKQGGSGGAPHH
HHHH
Anti-GD2 × murine anti-DOTA P53 SADA (SS) polypeptide (hu3F8-scFv, GS linker, C825-
scFv, (IgG3 spacer), huP53-tet)
(SEQ ID NO: 32)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SQVQLVESGPGVVQPGRSLRISCAVSGFSVTNYGVHWVRQPPGKCLEWLGVIWAGGIT
NYNSAFMSRLTISKDNSKNTVYLQMNSLRAEDTAMYYCASRGGHYGYALDYWGQGT
LVYVSSGGGGSGGGGSGGGGSGGGGSHVKLQESGPGLVQPSQSLSLTCTVSGFSLTDYGV
HWVRQSPGKGLEWLGVIWSGGGTAYNTALISRLNIYRDNSKNQVFLEMNSLQAEDTAMYYCA
RRGSYPYNYFDAWGCGTTVTVSSGGGGSGGGGSGGGGSQAVVIQESALTTPPGETVTLTCGS
STGAVTASNYANWVQEKPDHCFTGLIGGHNNRPPGVPARFSGSLIGDKAALTIAGTQTEDEAI
YFCALWYSDHWVIGGGTRLTVLG(TPLGDTTHT)SGKPLDGEYFTLQIRGRERFEMFREL
NEALELKDAQAGKEPGGSGGAPHHHHHH
Anti-GD2 × murine anti-DOTA P63 SADA (SS) polypeptide (hu3F8-scFv, GS linker, C825-
scFv, (IgG3 spacer), huP63-tet)
(SEQ ID NO: 33)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SQVQLVESGPGVVQPGRSLRISCAVSGFSVTNYGVHWVRQPPGKCLEWLGVIWAGGIT
NYNSAFMSRLTISKDNSKNTVYLQMNSLRAEDTAMYYCASRGGHYGYALDYWGQGT
LVTVSSGGGGSGGGGSGGGGSGGGGSHVKLQESGPGLVQPSQSLSLTCTVSGFSLTDYGV
HWVRQSPGKGLEWLGVIWSGGGTAYNTALISRLNIYRDNSKNQVFLEMNSLQAEDTAMYYCA
RRGSYPYNYFDAWGCGTTVTVSSGGGGSGGGGSGGGGSQAVVIQESALTTPPGETVTLTCGS
STGAVTASNYANWVQEKPDHCFTGLIGGHNNRPPGVPARFSGSLIGDKAALTIAGTQTEDEAI
YFCALWYSDHWVIGGGTRLTVLG(TPLGDTTHT)SGRSPDDELLYLPVRGRETYEMLLKI
KESLELMQYLPQHTIETYRQQQQQQHQHLLQKQGGSGGAPHHHHHH
Anti-GD2 × murine anti-DOTA P53 SADA (LL) polypeptide (hu3F8-scFv, GS linker, C825-
scFv, (IgG3 spacer), huP53-tet)
(SEQ ID NO: 34)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGOGTLVTVSSGGGGSGGGGSGGGGSGGGGSHVKLQESGPGLVQPSQSLS
LTCTVSGFSLTDYGVHWVRQSPGKGLEWLGVIWSGGGTAYNTALISRLNIYRDNSKNQVFLEM
NSLQAEDTAMYYCARRGSYPYNYFDAWGCGTTVTVSSGGGGSGGGGSGGGGSGGGGSGGG
GSGGGGSQAVVIQESALTTPPGETVTLTCGSSTGAVTASNYANWVQEKPDHCFTGLIGGHNN
RPPGVPARFSGSLIGDKAALTIAGTQTEDEAIYFCALWYSDHWVIGGGTRLTVLG(TPLGDTTH
T)SGKPLDGEYFTLQIRGRERFEMFRELNEALELKDAQAGKEPGGSGGAPHHHHHH
Anti-GD2 × murine anti-DOTA P63 SADA (LL) polypeptide (hu3F8-scFv, GS linker, C825-
scFv, (IgG3 spacer), huP63-tet)
(SEQ ID NO: 35)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGQGTLVTVSSGGGGSGGGGSGGGGSGGGGSHVKLQESGPGLVQPSQSLS
LTCTVSGFSLTDYGVHWVRQSPGKGLEWLGVIWSGGGTAYNTALISRLNIYRDNSKNQVFLEM
NSLQAEDTAMYYCARRGSYPYNYFDAWGCGTTVTVSSGGGGSGGGGSGGGGSGGGGSGGG
GSGGGGSQAVVIQESALTTPPGETVTLTCGSSTGAVTASNYANWVQEKPDHCFTGLIGGHNN
RPPGVPARFSGSLIGDKAALTIAGTQTEDEAIYFCALWYSDHWVIGGGTRLTVLG(TPLGDTTH
T)SGRSPDDELLYLPVRGRETYEMLLKIKESLELMQYLPQHTIETYRQQQQQQHQH
LLQKQGGSGGAPHHHHHH
Anti-GD2 × anti-DOTA P63 SADA (LL) (noHIS) polypeptide (hu3F8-scFv, GS linker, huC825-
scFv, (IgG3 spacer), huP63-tet)
(SEQ ID NO: 38)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGOGTLVTVSSGGGGSGGGGSGGGGSGGGGSHVQLVESGGGLVQPGGSL
RLSCAASGFSLTDYGVHWVRQAPGKGLEWLGVIWSGGGTAYNTALISRFTISRDNSKNTLYLQ
MNSLRAEDTAVYYCARRGSYPYNYFDAWGCGTLVTVSSGGGGSGGGGSGGGGSGGGGSGG
GGSGGGGSQAVVTQEPSLTVSPGGTVTLTCGSSTGAVTASNYANWVQQKPGQCPRGLIGGH
NNRPPGVPARFSGSLLGGKAALTLLGAQPEDEAEYYCALWYSDHWVIGGGTKLTVLG(TPLG
DTTHT)SGRSPDDELLYLPVRGRETYEMLLKIKESLELMQYLPQHTIETYRQQQQQ
QHQHLLQKQGGSGGA
Anti-GD2 × anti-DOTA P53 SADA (LL) (noHIS) polypeptide (hu3F8-scFv, GS linker, huC825-
scFv, (IgG3 spacer), huP53-tet)
(SEQ ID NO: 39)
EIVMTQTPATLSVSAGERVTITCKASQSVSNDVTWYQQKPGQAPRLLIYSASNRYSGVP
ARFSGSGYGTEFTFTISSVQSEDFAVYFCQQDYSSFGCGTKLEIKRGGGGSGGGGSGGGG
SGGGGSGGGGSGGGGSQVQLVESGPGVVQPGRSLRISCAVSGFSVTNYGVHWVRQPPG
KCLEWLGVIWAGGITNYNSAFMSRLTISKDNSKNTVYLQMNSLRAEDTAMYYCASRGG
HYGYALDYWGOGTLVTVSSGGGGSGGGGSGGGGSGGGGSHVQLVESGGGLVQPGGSL
RLSCAASGFSLTDYGVHWVRQAPGKGLEWLGVIWSGGGTAYNTALISRFTISRDNSKNTLYLQ
MNSLRAEDTAVYYCARRGSYPYNYFDAWGCGTLVTVSSGGGGSGGGGSGGGGSGGGGSGG
GGSGGGGSQAVVTQEPSLTVSPGGTVTLTCGSSTGAVTASNYANWVQQKPGQCPRGLIGGH
NNRPPGVPARFSGSLLGGKAALTLLGAQPEDEAEYYCALWYSDHWVIGGGTKLTVLG(TPLG
DTTHT)SGKPLDGEYFTLQIRGRERFEMFRELNEALELKDAQAGKEPGGSGGA

Conjugate Production. The anti-GD2 SADA conjugates described herein may be produced from nucleic acid molecules using molecular biological methods known in the art. Nucleic acid molecules are inserted into a vector that is able to express the fusion proteins when introduced into an appropriate host cell. Appropriate host cells include, but are not limited to, bacterial, yeast, insect, and mammalian cells. Any of the methods known to one skilled in the art for the insertion of DNA fragments into a vector may be used to construct expression vectors encoding the anti-GD2 SADA conjugates of the present technology under control of transcriptional/translational control signals. These methods may include in vitro recombinant DNA and synthetic techniques and in vivo recombination (See Sambrook et al. Molecular Cloning, A Laboratory Manual, Cold Spring Harbor Laboratory; Current Protocols in Molecular Biology, Eds. Ausubel, et al, Greene Publ. Assoc., Wiley-Interscience, NY).

Expression of nucleic acid molecules encoding the anti-GD2 SADA conjugates of the present technology may be regulated by a second nucleic acid sequence so that the molecule is expressed in a host transformed with the recombinant DNA molecule. For example, expression of the nucleic acid molecules encoding the anti-GD2 SADA conjugates of the present technology may be controlled by a promoter and/or enhancer element that are known in the art.

Nucleic acid constructs include sequences that encode anti-GD2 SADA conjugates that include a SADA domain, a GD2-specific antigen binding domain, and a DOTA-specific antigen binding domain. Typically, such antigen binding domains will be generated from V_{H} and/or V_{L} regions. After identification and selection of antigen binding domains exhibiting desired binding and/or functional properties, variable regions of each antigen binding domain are isolated, amplified, cloned and sequenced. Modifications may be made to the V_{H} and V_{L} nucleotide sequences, including additions of nucleotide sequences encoding amino acids and/or carrying restriction sites, deletions of nucleotide sequences encoding amino acids, or substitutions of nucleotide sequences encoding amino acids. The antigen binding domains may be generated from human, humanized or chimeric antibodies.

Nucleic acid constructs encoding the anti-GD2 SADA conjugates of the present technology are inserted into an expression vector or viral vector by methods known in the art, and nucleic acid molecules are operatively linked to an expression control sequence.

Where appropriate, nucleic acid sequences that encode the anti-GD2 SADA conjugates as described herein may be modified to include codons that are optimized for expression in a particular cell type or organism (e.g., see U.S. Pat. Nos. 5,670,356 and 5,874,304). Codon optimized sequences are synthetic sequences, and preferably encode the identical polypeptide (or a biologically active fragment of a full length polypeptide which has substantially the same activity as the full length polypeptide) encoded by the non-codon optimized parent polynucleotide. In some embodiments, the coding region of the genetic material encoding antibody components, in whole or in part, may include an altered sequence to optimize codon usage for a particular cell type (e.g., a eukaryotic or prokaryotic cell). For example, the coding sequence for a humanized heavy (or light) chain variable region as described herein may be optimized for expression in a bacterial cells. Alternatively, the coding sequence may be optimized for expression in a mammalian cell (e.g., a CHO). Such a sequence may be described as a codon-optimized sequence.

An expression vector containing a nucleic acid molecule is transformed into a suitable host cell to allow for production of the protein encoded by the nucleic acid constructs. Exemplary host cells include prokaryotes (e.g., E. coli) and eukaryotes (e.g., a COS or CHO cell). Host cells transformed with an expression vector are grown under conditions permitting production of anti-GD2 SADA conjugate of the present technology followed by recovery of the anti-GD2 SADA conjugate.

Anti-GD2 SADA conjugates of the present disclosure may be purified by any technique. For example, anti-GD2 SADA conjugates may be recovered from cells either as soluble polypeptides or as inclusion bodies, from which they may be extracted quantitatively by 8M guanidinium hydrochloride and dialysis. In order to further purify anti-GD2 SADA conjugates of the present technology, conventional ion exchange chromatography, hydrophobic interaction chromatography, reverse phase chromatography or gel filtration may be used. Anti-GD2 SADA conjugates of the present technology may also be recovered from conditioned media following secretion from eukaryotic or prokaryotic cells.

In some embodiments, as will be understood in the art, an anti-GD2 SADA conjugate may be utilized without further modification. In some embodiments, an anti-GD2 SADA conjugate may be incorporated into a composition or formulation.

A variety of technologies for conjugating agents, or components thereof, with other moieties or entities are well known in the art and may be utilized in accordance with the practice of the present disclosure. To give but one example, radioactively-labeled anti-GD2 SADA conjugates may be produced according to well-known technologies in the art. For instance, in some embodiments, anti-GD2 SADA conjugates can be iodinated by contact with sodium and/or potassium iodide and a chemical oxidizing agent such as sodium hypochlorite, or an enzymatic oxidizing agent, such as lactoperoxidase. In some embodiments, anti-GD2 SADA conjugates may be labeled with technetium-99m by ligand exchange process, for example, by reducing pertechnate with stannous solution, chelating the reduced technetium onto a Sephadex column and applying the anti-GD2 SADA conjugate to the column. In some embodiments, anti-GD2 SADA conjugates of the present technology are labeled using direct labeling techniques, e.g., by incubating pertechnate, a reducing agent such as SNCl₂, a buffer solution such as sodium-potassium phthalate solution, and the anti-GD2 SADA conjugate. Intermediary functional groups which are often used to bind radioisotopes which exist as metallic ions to anti-GD2 SADA conjugates are diethylenetriaminepentaacetic acid (DTPA), or ethylene diaminetetracetic acid (EDTA), or 1,4,7,10-tetraazacyclododecane-1,4,7,10-tetraacetic acid (DOTA), or p-aminobenzyl-DOTA (Bn-DOTA). Radioactive isotopes may be detected by, for example, dosimetry.

### Therapeutic Use of the Anti-GD2 SADA Conjugates of the Present Technology

In one aspect, the anti-GD2 SADA conjugate compositions of the present technology (e.g., any of the anti-GD2×anti DOTA antigen binding fragments thereof described herein) are useful for the treatment of GD2-associated cancers. Such treatment can be used in patients identified as having pathologically high levels of the GD2 (e.g., those diagnosed by conventional detection methods known in the art) or in patients diagnosed with a disease known to be associated with such pathological levels. Examples of GD2-associated cancers that can be treated by the anti-GD2 SADA conjugate compositions of the present technology include, but are not limited to: neuroblastoma, melanoma, soft tissue sarcoma, brain tumor, osteosarcoma, small-cell lung cancer, breast cancer, or retinoblastoma. In some embodiments, the soft tissue sarcoma is liposarcoma, fibrosarcoma, malignant fibrous histiocytoma, leimyosarcoma, or spindle cell sarcoma.

The compositions of the present technology may be employed in conjunction with other therapeutic agents useful in the treatment of GD2-associated cancers. For example, the anti-GD2 SADA conjugates of the present technology may be separately, sequentially or simultaneously administered with at least one additional therapeutic agent-selected from the group consisting of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.) nanoparticles, liposomes, other DOTA-haptens (Proteus-like, etc). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

The anti-GD2 SADA conjugate compositions of the present technology may optionally be administered as a single dose to a subject in need thereof. Alternatively, the dosing regimen may comprise multiple administrations performed at various times after the appearance of tumors. Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved. In some embodiments, the anti-GD2 SADA conjugate compositions of the present technology comprise pharmaceutical formulations which may be administered to subjects in need thereof in one or more doses. Dosage regimens can be adjusted to provide the desired response (e.g., a therapeutic response).

Typically, an effective amount of the anti-GD2 SADA conjugate compositions of the present technology, sufficient for achieving a therapeutic effect, ranges from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of anti-GD2 SADA conjugate, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of anti-GD2 SADA conjugate ranges from 0.1-10,000 micrograms per kg body weight. In one embodiment, anti-GD2 SADA conjugate concentrations in a carrier range from 0.2 to 2000 micrograms per delivered milliliter. Anti-GD2 SADA conjugates may be administered on multiple occasions. Intervals between single dosages can be hourly, daily, weekly, monthly or yearly. Intervals can also be irregular as indicated by measuring blood levels of the anti-GD2 SADA conjugate in the subject. In some methods, dosage is adjusted to achieve a serum anti-GD2 SADA conjugate concentration in the subject of from about 75 μg/mL to about 125 μg/mL, 100 μg/mL to about 150 μg/mL, from about 125 g/mL to about 175 μg/mL, or from about 150 μg/mL to about 200 μg/mL. Alternatively, anti-GD2 SADA conjugate can be administered as a sustained release formulation, in which case less frequent administration is required. Dosage and frequency vary depending on the half-life of the anti-GD2 SADA conjugate in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

PRIT. In one aspect, the present disclosure provides a method for detecting tumors in a subject in need thereof comprising (a) administering to the subject an effective amount of an anti-GD2 SADA conjugate of the present technology that is capable of binding to a DOTA hapten, and a GD2 antigen, wherein the anti-GD2 SADA conjugate is configured to localize to a tumor expressing the GD2 antigen recognized by the anti-GD2 SADA conjugate; (b) administering to the subject an effective amount of a radiolabeled DOTA hapten, wherein the radiolabeled DOTA hapten is configured to bind to the anti-GD2 SADA conjugate; and (c) detecting the presence of tumors in the subject by detecting radioactive levels emitted by the anti-GD2 SADA conjugate that are higher than a reference value. In some embodiments, the subject is human. Additionally or alternatively, in some embodiments, the radiolabel is an alpha particle-emitting isotope, a beta particle-emitting isotope, an Auger-emitter, or any combination thereof. Examples of beta particle-emitting isotopes include 86Y ⁹⁰Y, ⁸⁹Sr, ¹⁶⁵Dy, ¹⁸⁶Re, ¹⁸⁸Re, ¹⁷⁷Lu, and ⁶⁷Cu. Examples of alpha particle-emitting isotopes include ²¹³Bi, ²¹¹At, ²²⁵Ac, ¹⁵²Dy, ²¹²Bi, ²²³Ra, ²¹⁹Rn ²¹⁵Po, ²¹¹Bi, ²²¹Fr, ²¹⁷At, and ²⁵⁵Fm. Examples of Auger-emitters include ¹¹¹In, ⁶⁷Ga, ⁵¹Cr, ⁵⁸Co, ⁹⁹mTc, ^{103m}Rh, ^{195m}Pt, ¹¹⁹Sb, ¹⁶¹Ho, ^{189m}Os, ¹⁹²Ir, ²⁰¹Tl, and ²⁰³Pb. Additionally or alternatively, in some embodiments of the methods disclosed herein, the radioactive levels emitted by the anti-GD2 SADA conjugate are detected using positron emission tomography or single photon emission computed tomography.

In one aspect, the present disclosure provides a method for selecting a subject for pretargeted radioimmunotherapy comprising (a) administering to the subject an effective amount of an anti-GD2 SADA conjugate of the present technology that is capable of binding to a DOTA hapten, and a GD2 antigen, wherein the anti-GD2 SADA conjugate is configured to localize to a tumor expressing the GD2 antigen recognized by the anti-GD2 SADA conjugate; (b) administering to the subject an effective amount of a radiolabeled DOTA hapten, wherein the radiolabeled DOTA hapten is configured to bind to the anti-GD2 SADA conjugate; (c) detecting radioactive levels emitted by the anti-GD2 SADA conjugate; and (d) selecting the subject for pretargeted radioimmunotherapy when the radioactive levels emitted by the anti-GD2 SADA conjugate are higher than a reference value. In some embodiments, the subject is human.

In any of the preceding embodiments of the methods disclosed herein, the DOTA haptens is selected from the group consisting of (i) DOTA-Phe-Lys(HSG)-D-Tyr-Lys(HSG)-NH₂; (ii) Ac-Lys(HSG)D-Tyr-Lys(HSG)-Lys(Tscg-Cys)-NH₂; (iii) DOTA-D-Asp-D-Lys(HSG)-D-Asp-D-Lys(HSG)-NH₂; (iv) DOTA-D-Glu-D-Lys(HSG)-D-Glu-D-Lys(HSG)-NH₂; (v) DOTA-D-Tyr-D-Lys(HSG)-D-Glu-D-Lys(HSG)-NH₂; (vi) DOTA-D-Ala-D-Lys(HSG)-D-Glu-D-Lys(HSG)-NH₂; (vii) DOTA-D-Phe-D-Lys(HSG)-D-Tyr-D-Lys(HSG)-NH₂; (viii) Ac-D-Phe-D-Lys(DOTA)-D-Tyr-D-Lys(DOTA)-NH₂; (ix) Ac-D-Phe-D-Lys(DTPA)-D-Tyr-D-Lys(DTPA)-NH₂; (x) Ac-D-Phe-D-Lys(Bz-DTPA)-D-Tyr-D-Lys(Bz-DTPA)-NH₂; (xi) Ac-D-Lys(HSG)-D-Tyr-D-Lys(HSG)-D-Lys(Tscg-Cys)-NH₂; (xii) DOTA-D-Phe-D-Lys(HSG)-D-Tyr-D-Lys(HSG)-D-Lys(Tscg-Cys)-NH₂; (xiii) (Tscg-Cys)-D-Phe-D-Lys(HSG)-D-Tyr-D-Lys(HSG)-D-Lys(DOTA)-NH₂; (xiv) Tscg-D-Cys-D-Glu-D-Lys(HSG)-D-Glu-D-Lys(HSG)-NH₂; (xv) (Tscg-Cys)-D-Glu-D-Lys(HSG)-D-Glu-D-Lys(HSG)-NH₂; (xvi) Ac-D-Cys-D-Lys(DOTA)-D-Tyr-D-Ala-D-Lys(DOTA)-D-Cys-NH₂; (xvii) Ac-D-Cys-D-Lys(DTPA)-D-Tyr-D-Lys(DTPA)-NH₂; (xviii) Ac-D-Lys(DTPA)-D-Tyr-D-Lys(DTPA)-D-Lys(Tscg-Cys)-NH₂; (xix) Ac-D-Lys(DOTA)-D-Tyr-D-Lys(DOTA)-D-Lys(Tscg-Cys)-NH₂, (xx) DOTA and (xxi) Proteus-DOTA. The radiolabel may be an alpha particle-emitting isotope, a beta particle-emitting isotope, or an Auger-emitter. Examples of radiolabels include ²¹³Bi, ²¹¹At, ²²⁵Ac, ¹⁵²Dy, ²¹²Bi, ²²³Ra, ²¹⁹R, ²¹⁵P, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ⁹⁹mTc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu.

Additionally or alternatively, in some embodiments of the methods disclosed herein, the subject is diagnosed with, or is suspected of having a GD2-associated cancer such as neuroblastoma, melanoma, brain tumor, osteosarcoma, small-cell lung cancer, retinoblastoma, liposarcoma, fibrosarcoma, malignant fibrous histiocytoma, leimyosarcoma, breast cancer, or spindle cell sarcoma.

Additionally or alternatively, in some embodiments of the methods disclosed herein, the anti-GD2 SADA conjugate and/or the radiolabeled DOTA hapten is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the anti-GD2 SADA conjugate and/or the radiolabeled DOTA hapten is administered into the cerebral spinal fluid or blood of the subject.

In some embodiments of the methods disclosed herein, the radioactive levels emitted by the anti-GD2 SADA conjugate are detected between 2 to 120 hours after the radiolabeled DOTA hapten is administered. In certain embodiments of the methods disclosed herein, the radioactive levels emitted by the anti-GD2 SADA conjugate are expressed as the percentage injected dose per gram tissue (% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45(9):1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

In one aspect, the present disclosure provides a method for reducing or mitigating alpha-radioimmunotherapy-associated toxicity in a subject in need thereof comprising administering to the subject an effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; and administering to the subject an effective amount of a DOTA hapten comprising an alpha particle-emitting isotope, wherein the DOTA hapten is configured to bind to the anti-GD2 SADA conjugate. In certain embodiments, the subject has received or is receiving one or more cycles of alpha-radioimmunotherapy. Examples of alpha particle-emitting isotopes include, but are not limited to, ²¹³Bi, ²¹¹At, ²²⁵Ac, ¹⁵²Dy, ²¹²Bi, ²²³Ra, ²¹⁹Rn, ²¹⁵Po ²¹¹Bi, ²²Fr, ²¹⁷At, or ²⁵⁵Fm. The alpha-radioimmunotherapy-associated toxicity may be toxicity to one or more organs selected from the group consisting of brain, kidney, bladder, liver, bone marrow and spleen. In some embodiments, the subject is human.

In another aspect, the present disclosure provides a method for increasing the efficacy of beta-radioimmunotherapy in a subject in need thereof comprising (a) administering to the subject an effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 36-96 hours (e.g., about 48 hours) after administration of the anti-GD2 SADA conjugate, wherein the DOTA hapten (i) comprises a beta particle-emitting isotope, and (ii) is configured to bind to the anti-GD2 SADA conjugate; (c) administering to the subject a second dose of the DOTA hapten about 24 hours after administration of the first dose of the DOTA hapten; and (d) administering to the subject a third dose of the DOTA hapten about 24 hours after administration of the second dose of the DOTA hapten. In some embodiments, the radiolabeled-DOTA hapten are administered without further administration of the anti-GD2 SADA conjugate of the present technology. In other embodiments, the method further comprises repeating steps (a)-(d) for at least 1, 2, 3, 4, 5, 6, 7, 8, 9, 10 or more additional cycles. In some embodiments, the subject is human. Additionally or alternatively, in some embodiments of the methods disclosed herein, the effective amount of the anti-GD2 SADA conjugate may be about 0.5 mg/kg to about 400 mg/kg. Additionally or alternatively, in some embodiments of the methods of the present technology, the effective amount of the anti-GD2 SADA conjugate is about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, or about 400 mg/kg. Values and ranges intermediate to the recited values are also contemplated. In any of the preceding embodiments of the methods disclosed herein, the first, second, and/or third doses of the DOTA hapten may be 50 pmol-500 pmol per gram of tumor. Additionally or alternatively, in some embodiments of the methods of the present technology, the first, second, and/or third doses of the DOTA hapten is about 50 pmol/g of tumor, about 55 pmol/g of tumor, about 60 pmol/g of tumor, about 65 pmol/g of tumor, about 70 pmol/g of tumor, about 75 pmol/g of tumor, about 80 pmol/g of tumor, about 85 pmol/g of tumor, about 90 pmol/g of tumor, about 95 pmol/g of tumor, about 100 pmol/g of tumor, about 125 pmol/g of tumor, about 150 pmol/g of tumor, about 175 pmol/g of tumor, about 200 pmol/g of tumor, about 225 pmol/g of tumor, about 250 pmol/g of tumor, about 275 pmol/g of tumor, about 300 pmol/g of tumor, about 325 pmol/g of tumor, about 350 pmol/g of tumor, about 375 pmol/g of tumor, about 400 pmol/g of tumor, about 425 pmol/g of tumor, about 450 pmol/g of tumor, about 475 pmol/g of tumor, or about 500 pmol/g of tumor. Values and ranges intermediate to the recited values are also contemplated. In certain embodiments, the first, second, and/or third doses of the DOTA hapten is about 50 pmol to 10 nmol (e.g., 50 pmol, 60 pmol, 70 pmol, 80 pmol, 90 pmol, 100 pmol, 200 pmol, 300 pmol, 400 pmol, 500 pmol, 600 pmol, 700 pmol, 800 pmol, 900 pmol, 1 nmol, 2 nmol, 3 nmol, 4 nmol, 5 nmol, 6 nmol, 7 nmol, 8 nmol, 9 nmol, 10 nmol). Values and ranges intermediate to the recited values are also contemplated. Additionally or alternatively, in some embodiments of the methods disclosed herein, the first, second, and third doses of the DOTA hapten are identical. In other embodiments of the methods disclosed herein, any two of the first, second, and third doses of the DOTA hapten may be identical. In certain embodiments of the methods disclosed herein, the first, second, and third doses of the DOTA hapten are different. In any of the preceding embodiments of the methods disclosed herein, the beta particle-emitting isotope is ⁸⁶Y, ⁹⁰Y ⁸⁹Sr, ¹⁶⁵Dy, ¹⁸⁶Re, ¹⁸⁸Re, ¹⁷⁷Lu, or ⁶⁷Cu.

In yet another aspect, the present disclosure provides a method for increasing the efficacy of beta-radioimmunotherapy in a subject in need thereof comprising (a) administering to the subject a first effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 36-96 hours (e.g., about 48 hours) after administration of the first effective amount of the anti-GD2 SADA conjugate, wherein the DOTA hapten (i) comprises a beta particle-emitting isotope, and (ii) is configured to bind to the anti-GD2 SADA conjugate; (c) administering to the subject a second effective amount of the anti-GD2 SADA conjugate about 7 days after administration of the first effective amount of the anti-GD2 SADA conjugate; (d) administering to the subject a second dose of the DOTA hapten about 36-96 hours (e.g., about 48 hours) after administration of the second effective amount of the anti-GD2 SADA conjugate; (e) administering to the subject a third effective amount of the anti-GD2 SADA conjugate about 7 days after administration of the second effective amount of the anti-GD2 SADA conjugate; and (f) administering to the subject a third dose of the DOTA hapten about 36-96 hours (e.g., about 48 hours) after administration of the third effective amount of the anti-GD2 SADA conjugate. In some embodiments, the subject is human. In any and all embodiments of the methods disclosed herein, the first, second, and/or third effective amounts of the anti-GD2 SADA conjugate may be about 0.5 mg/kg to about 400 mg/kg. Additionally or alternatively, in some embodiments, the first, second, and/or third effective amounts of the anti-GD2 SADA conjugate is about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, or about 400 mg/kg. Values and ranges intermediate to the recited values are also contemplated. Additionally or alternatively, in some embodiments of the methods disclosed herein, the first, second, and third effective amounts of the anti-GD2 SADA conjugate are identical. In other embodiments of the methods disclosed herein, any two of the first, second, and third effective amounts of the anti-GD2 SADA conjugate may be identical. In certain embodiments of the methods disclosed herein, the first, second, and third effective amounts of the anti-GD2 SADA conjugate are different. In any of the preceding embodiments of the methods disclosed herein, the first, second, and/or third doses of the DOTA hapten may be 50 pmol-500 pmol per gram of tumor. Additionally or alternatively, in some embodiments of the methods of the present technology, the first, second, and/or third doses of the DOTA hapten is about 50 pmol/g of tumor, about 55 pmol/g of tumor, about 60 pmol/g of tumor, about 65 pmol/g of tumor, about 70 pmol/g of tumor, about 75 pmol/g of tumor, about 80 pmol/g of tumor, about 85 pmol/g of tumor, about 90 pmol/g of tumor, about 95 pmol/g of tumor, about 100 pmol/g of tumor, about 125 pmol/g of tumor, about 150 pmol/g of tumor, about 175 pmol/g of tumor, about 200 pmol/g of tumor, about 225 pmol/g of tumor, about 250 pmol/g of tumor, about 275 pmol/g of tumor, about 300 pmol/g of tumor, about 325 pmol/g of tumor, about 350 pmol/g of tumor, about 375 pmol/g of tumor, about 400 pmol/g of tumor, about 425 pmol/g of tumor, about 450 pmol/g of tumor, about 475 pmol/g of tumor, or about 500 pmol/g of tumor. Values and ranges intermediate to the recited values are also contemplated. In certain embodiments, the first, second, and/or third doses of the DOTA hapten is about 50 pmol to 10 nmol (e.g., 50 pmol, 60 pmol, 70 pmol, 80 pmol, 90 pmol, 100 pmol, 200 pmol, 300 pmol, 400 pmol, 500 pmol, 600 pmol, 700 pmol, 800 pmol, 900 pmol, 1 nmol, 2 nmol, 3 nmol, 4 nmol, 5 nmol, 6 nmol, 7 nmol, 8 nmol, 9 nmol, 10 nmol). Values and ranges intermediate to the recited values are also contemplated. Additionally or alternatively, in some embodiments of the methods disclosed herein, the first, second, and third doses of the DOTA hapten are identical. In other embodiments of the methods disclosed herein, any two of the first, second, and third doses of the DOTA hapten may be identical. In certain embodiments of the methods disclosed herein, the first, second, and third doses of the DOTA hapten are different. In any of the preceding embodiments of the methods disclosed herein, the beta particle-emitting isotope is ⁸⁶Y, ⁹⁰Y ⁸⁹Sr, ¹⁶⁵Dy, ¹⁸⁶Re, ¹⁸⁸Re, ¹⁷⁷Lu, or ⁶⁷Cu.

The anti-GD2 SADA conjugate is administered under conditions and for a period of time (e.g., according to a dosing regimen) sufficient for it to saturate tumor cells. In some embodiments, unbound anti-GD2 SADA conjugate is cleared from the blood stream after administration of the anti-GD2 SADA conjugate. In some embodiments, the radiolabeled-DOTA hapten is administered after a time period that may be sufficient to permit clearance of unbound anti-GD2 SADA conjugate.

The radiolabeled-DOTA hapten may be administered at any time between 1.5 to 4 days following administration of the anti-GD2 SADA conjugate. For example, in some embodiments, the radiolabeled-DOTA hapten is administered 36 hours, 48 hours, 96 hours, or any range therein, following administration of the anti-GD2 SADA conjugate.

The therapeutic effectiveness of such an anti-GD2 SADA conjugate described herein may be determined by computing the area under the curve (AUC) tumor: AUC normal tissue ratio. In some embodiments, the anti-GD2 SADA conjugate has a AUC tumor: AUC normal tissue ratio of about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

Additionally or alternatively, in some embodiments of the preceding methods disclosed herein, the anti-GD2 SADA conjugate and/or the radiolabeled-DOTA hapten is administered intravenously, intramuscularly, intraarterially, intrathecally, intracapsularly, intraorbitally, intradermally, intraperitoneally, transtracheally, subcutaneously, intracerebroventricularly, intratumorally, orally or intranasally.

In one aspect, the present disclosure provides a method for treating a GD2-associated cancer in a subject in need thereof comprising (a) administering to the subject an effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 36-96 hours (e.g., about 48 hours) after administration of the anti-GD2 SADA conjugate, wherein the DOTA hapten (i) comprises a beta particle-emitting isotope or an alpha particle-emitting isotope, and (ii) is configured to bind to the anti-GD2 SADA conjugate; (c) administering to the subject a second dose of the DOTA hapten about 24 hours after administration of the first dose of the DOTA hapten; and (d) administering to the subject a third dose of the DOTA hapten about 24 hours after administration of the second dose of the DOTA hapten. In some embodiments, the radiolabeled-DOTA hapten are administered without further administration of the anti-GD2 SADA conjugate of the present technology. In other embodiments, the method further comprises repeating steps (a)-(d) for at least 1, 2, 3, 4, 5, 6, 7, 8, 9, 10 or more additional cycles. In some embodiments, the subject is human. Additionally or alternatively, in some embodiments of the methods disclosed herein, the effective amount of the anti-GD2 SADA conjugate may be about 0.5 mg/kg to about 400 mg/kg. Additionally or alternatively, in some embodiments, the effective amount of the anti-GD2 SADA conjugate is about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, or about 400 mg/kg. Values and ranges intermediate to the recited values are also contemplated. In any of the preceding embodiments of the methods disclosed herein, the first, second, and/or third doses of the DOTA hapten may be 50 pmol-500 pmol per gram of tumor. Additionally or alternatively, in some embodiments of the methods of the present technology, the first, second, and/or third doses of the DOTA hapten is about 50 pmol/g of tumor, about 55 pmol/g of tumor, about 60 pmol/g of tumor, about 65 pmol/g of tumor, about 70 pmol/g of tumor, about 75 pmol/g of tumor, about 80 pmol/g of tumor, about 85 pmol/g of tumor, about 90 pmol/g of tumor, about 95 pmol/g of tumor, about 100 pmol/g of tumor, about 125 pmol/g of tumor, about 150 pmol/g of tumor, about 175 pmol/g of tumor, about 200 pmol/g of tumor, about 225 pmol/g of tumor, about 250 pmol/g of tumor, about 275 pmol/g of tumor, about 300 pmol/g of tumor, about 325 pmol/g of tumor, about 350 pmol/g of tumor, about 375 pmol/g of tumor, about 400 pmol/g of tumor, about 425 pmol/g of tumor, about 450 pmol/g of tumor, about 475 pmol/g of tumor, or about 500 pmol/g of tumor. Values and ranges intermediate to the recited values are also contemplated. In certain embodiments, the first, second, and/or third doses of the DOTA hapten is about 50 pmol to 10 nmol (e.g., 50 pmol, 60 pmol, 70 pmol, 80 pmol, 90 pmol, 100 pmol, 200 pmol, 300 pmol, 400 pmol, 500 pmol, 600 pmol, 700 pmol, 800 pmol, 900 pmol, 1 nmol, 2 nmol, 3 nmol, 4 nmol, 5 nmol, 6 nmol, 7 nmol, 8 nmol, 9 nmol, 10 nmol). Values and ranges intermediate to the recited values are also contemplated. Additionally or alternatively, in some embodiments of the methods disclosed herein, the first, second, and third doses of the DOTA hapten are identical. In other embodiments of the methods disclosed herein, any two of the first, second, and third doses of the DOTA hapten may be identical. In certain embodiments of the methods disclosed herein, the first, second, and third doses of the DOTA hapten are different. In any of the preceding embodiments of the methods disclosed herein, the beta particle-emitting isotope is ⁸⁶Y, ⁹⁰Y, ⁸⁹Sr, ¹⁶⁵Dy, ¹⁸⁶Re, ¹⁸⁸Re, ¹⁷⁷Lu, or ⁶⁷Cu. Examples of the alpha particle-emitting isotope include ²¹³Bi, ²¹¹At, ²²⁵Ac, ¹⁵²Dy, ²¹²Bi, ²²³Ra, ²¹⁹R ²¹⁵Po, ²¹¹Bi, ²²¹Fr, ²¹⁷At, or ²⁵⁵Fm.

In another aspect, the present disclosure provides a method for treating a GD2-associated cancer in a subject in need thereof comprising (a) administering to the subject a first effective amount of an anti-GD2 SADA conjugate of the present technology comprising a self-assembly disassembly (SADA) polypeptide of p53 or p63, a GD2-specific antigen binding domain, and a DOTA-specific antigen binding domain, wherein the anti-GD2 SADA conjugate is configured to localize to a tumor expressing GD2; (b) administering to the subject a first dose of a DOTA hapten about 36-96 hours (e.g., about 48 hours) after administration of the first effective amount of the anti-GD2 SADA conjugate, wherein the DOTA hapten (i) comprises a beta particle-emitting isotope or an alpha particle-emitting isotope, and (ii) is configured to bind to the anti-GD2 SADA conjugate; (c) administering to the subject a second effective amount of the anti-GD2 SADA conjugate about 7 days after administration of the first effective amount of the anti-GD2 SADA conjugate; (d) administering to the subject a second dose of the DOTA hapten about 36-96 hours (e.g., about 48 hours) after administration of the second effective amount of the anti-GD2 SADA conjugate; (e) administering to the subject a third effective amount of the anti-GD2 SADA conjugate about 7 days after administration of the second effective amount of the anti-GD2 SADA conjugate; and (f) administering to the subject a third dose of the DOTA hapten about 36-96 hours (e.g., about 48 hours) after administration of the third effective amount of the anti-GD2 SADA conjugate. In some embodiments, the subject is human. In any and all embodiments of the methods disclosed herein, the first, second, and/or third effective amounts of the anti-GD2 SADA conjugate may be about 0.5 mg/kg to about 400 mg/kg. Additionally or alternatively, in some embodiments, the first, second, and/or third effective amounts of the anti-GD2 SADA conjugate is about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, or about 400 mg/kg. Values and ranges intermediate to the recited values are also contemplated. Additionally or alternatively, in some embodiments of the methods disclosed herein, the first, second, and third effective amounts of the anti-GD2 SADA conjugate are identical. In other embodiments of the methods disclosed herein, any two of the first, second, and third effective amounts of the anti-GD2 SADA conjugate may be identical. In certain embodiments of the methods disclosed herein, the first, second, and third effective amounts of the anti-GD2 SADA conjugate are different. In any of the preceding embodiments of the methods disclosed herein, the first, second, and/or third doses of the DOTA hapten may be 50 pmol-500 pmol per gram of tumor. Additionally or alternatively, in some embodiments of the methods of the present technology, the first, second, and/or third doses of the DOTA hapten is about 50 pmol/g of tumor, about 55 pmol/g of tumor, about 60 pmol/g of tumor, about 65 pmol/g of tumor, about 70 pmol/g of tumor, about 75 pmol/g of tumor, about 80 pmol/g of tumor, about 85 pmol/g of tumor, about 90 pmol/g of tumor, about 95 pmol/g of tumor, about 100 pmol/g of tumor, about 125 pmol/g of tumor, about 150 pmol/g of tumor, about 175 pmol/g of tumor, about 200 pmol/g of tumor, about 225 pmol/g of tumor, about 250 pmol/g of tumor, about 275 pmol/g of tumor, about 300 pmol/g of tumor, about 325 pmol/g of tumor, about 350 pmol/g of tumor, about 375 pmol/g of tumor, about 400 pmol/g of tumor, about 425 pmol/g of tumor, about 450 pmol/g of tumor, about 475 pmol/g of tumor, or about 500 pmol/g of tumor. Values and ranges intermediate to the recited values are also contemplated. In certain embodiments, the first, second, and/or third doses of the DOTA hapten is about 50 pmol to 10 nmol (e.g., 50 pmol, 60 pmol, 70 pmol, 80 pmol, 90 pmol, 100 pmol, 200 pmol, 300 pmol, 400 pmol, 500 pmol, 600 pmol, 700 pmol, 800 pmol, 900 pmol, 1 nmol, 2 nmol, 3 nmol, 4 nmol, 5 nmol, 6 nmol, 7 nmol, 8 nmol, 9 nmol, 10 nmol). Values and ranges intermediate to the recited values are also contemplated. Additionally or alternatively, in some embodiments of the methods disclosed herein, the first, second, and third doses of the DOTA hapten are identical. In other embodiments of the methods disclosed herein, any two of the first, second, and third doses of the DOTA hapten may be identical. In certain embodiments of the methods disclosed herein, the first, second, and third doses of the DOTA hapten are different. Examples of the beta particle-emitting isotope include ⁸⁶Y, ⁹⁰Y ⁸⁹Sr, ¹⁶⁵Dy, ¹⁸⁶Re, ¹⁸⁸Re, ¹⁷⁷Lu, or ⁶⁷Cu. Examples of the alpha particle-emitting isotope include ²¹³Bi, ²¹¹At, ²²⁵Ac, ¹⁵²Dy, ²¹²Bi, ²²³Ra, ²¹⁹Rn ²¹⁵Po, ²¹¹Bi, ²²Fr, ²¹⁷At, or ²⁵⁵Fm.

The anti-GD2 SADA conjugate is administered under conditions and for a period of time (e.g., according to a dosing regimen) sufficient for it to saturate tumor cells. In some embodiments, unbound anti-GD2 SADA conjugate is cleared from the blood stream after administration of the anti-GD2 SADA conjugate. In some embodiments, the radiolabeled-DOTA hapten is administered after a time period that may be sufficient to permit clearance of unbound anti-GD2 SADA conjugate.

The radiolabeled-DOTA hapten may be administered at any time between 1.5 to 4 days following administration of the anti-GD2 SADA conjugate. For example, in some embodiments, the radiolabeled-DOTA hapten is administered 36 hours, 48 hours, 96 hours, or any range therein, following administration of the anti-GD2 SADA conjugate.

In any and all embodiments of the methods disclosed herein, the subject suffers from or is diagnosed as having a GD2-associated cancer, such as neuroblastoma, melanoma, soft tissue sarcoma, brain tumor, osteosarcoma, small-cell lung cancer, retinoblastoma, liposarcoma, fibrosarcoma, malignant fibrous histiocytoma, leimyosarcoma, breast cancer, or spindle cell sarcoma.

In any of the above embodiments of the methods disclosed herein, the DOTA hapten is selected from the group consisting of DOTA, Proteus-DOTA, DOTA-Bn, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, and Ac-D-Lys(DOTA)-D-Tyr-D-Lys(DOTA)-D-Lys(Tscg-Cys)-NH₂.

Additionally or alternatively, in some embodiments of the methods disclosed herein, the administration of the anti-GD2 SADA conjugate results in decreased renal apoptosis in the subject compared to a GD2-associated cancer patient that has been treated with an anti-DOTA×anti-GD2 IgG-scFv-BsAb. In certain embodiments of the methods described herein, administration of the anti-GD2 SADA conjugate results in reduced immunogenicity in the subject compared to a GD2-associated cancer patient that has been treated with an anti-DOTA×anti-GD2 IgG-scFv-BsAb. Additionally or alternatively, in some embodiments of the methods disclosed herein, administration of the anti-GD2 SADA conjugate results in decreased severity of ovarian atrophy in the subject compared to a GD2-associated cancer patient that has been treated with an anti-DOTA×anti-GD2 IgG-scFv-BsAb. In some embodiments of the methods disclosed herein, administration of the anti-GD2 SADA conjugate results in prolonged remission in the subject compared to a GD2-associated cancer patient that has been treated with an anti-DOTA×anti-GD2 IgG-scFv-BsAb. In any of the preceding embodiments of the methods described herein, the anti-DOTA×anti-GD2 IgG-scFv-BsAb comprises (a) a GD2-specific antigen binding domain comprising a heavy chain variable domain (V_{H}) sequence and a light chain variable domain (V_{L}) sequence of SEQ ID NO: 1 and SEQ ID NO: 5, respectively, and (b) a DOTA-specific antigen binding domain comprising a heavy chain variable domain (V_{H}) sequence of SEQ ID NO: 9 or SEQ ID NO: 17, and a light chain variable domain (V_{L}) sequence of SEQ ID NO: 13 or SEQ ID NO: 18.

In any and all embodiments of the methods disclosed herein, administration of the anti-GD2 SADA conjugate results in decreased renal apoptosis, decreased severity of ovarian atrophy, and/or prolonged remission in the subject compared to a control GD2-associated cancer patient that does not receive the anti-GD2 SADA conjugate.

Toxicity. Optimally, an effective amount (e.g., dose) of an anti-GD2 SADA conjugate described herein will provide therapeutic benefit without causing substantial toxicity to the subject. Toxicity of the anti-GD2 SADA conjugate described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the anti-GD2 SADA conjugate described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

Formulations of Pharmaceutical Compositions. According to the methods of the present technology, the anti-GD2 SADA conjugate can be incorporated into pharmaceutical compositions suitable for administration. The pharmaceutical compositions generally comprise recombinant or substantially purified anti-GD2 SADA conjugate and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the anti-GD2 SADA conjugate compositions (See, e.g., Remington's Pharmaceutical Sciences, Mack Publishing Co., Easton, Pa. 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the anti-GD2 SADA conjugate, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. An anti-GD2 SADA conjugate named in this technology can be present in unsalified or unesterified form, or in salified and/or esterified form, and the naming of such anti-GD2 SADA conjugate is intended to include both the original (unsalified and unesterified) compound and its pharmaceutically-acceptable salts and esters. Also, certain embodiments of the present technology can be present in more than one stereoisomeric form, and the naming of such anti-GD2 SADA conjugate is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the anti-GD2 SADA conjugate, use thereof in the compositions is contemplated. Supplementary active compounds can also be incorporated into the compositions.

A pharmaceutical composition of the present technology is formulated to be compatible with its intended route of administration. The anti-GD2 SADA conjugate compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The anti-GD2 SADA conjugates can optionally be administered in combination with other agents that are at least partly effective in treating various GD2-associated cancers.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating an anti-GD2 SADA conjugate of the present technology in the required amount in an appropriate solvent with one or a combination of ingredients enumerated above, as required, followed by filtered sterilization. Generally, dispersions are prepared by incorporating the anti-GD2 SADA conjugate into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The anti-GD2 SADA conjugates of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the anti-GD2 SADA conjugate can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the anti-GD2 SADA conjugate is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the anti-GD2 SADA conjugate is formulated into ointments, salves, gels, or creams as generally known in the art.

The anti-GD2 SADA conjugate can also be prepared as pharmaceutical compositions in the form of suppositories (e.g., with conventional suppository bases such as cocoa butter and other glycerides) or retention enemas for rectal delivery.

In one embodiment, the anti-GD2 SADA conjugate is prepared with carriers that will protect the anti-GD2 SADA conjugate against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### C. Kits

The present technology provides kits for the detection and/or PRIT-related treatment of GD2-associated cancers, comprising at least one immunoglobulin-related composition of the present technology (e.g., any anti-GD2 SADA conjugate described herein), or a functional variant (e.g., substitutional variant) thereof. Optionally, the above described components of the kits of the present technology are packed in suitable containers and labeled for diagnosis and/or radioimmunotherapy-based treatment of GD2-associated cancers.

In one aspect, the kits comprise at least one anti-GD2 SADA conjugate (e.g., anti-DOTA bispecific antigen binding fragments) of the present technology, a DOTA hapten, and instructions for using the same in alpha- or beta-radioimmunotherapy (e.g., PRIT). Examples of suitable DOTA haptens include, but are not limited to, DOTA, Proteus-DOTA, DOTA-Bn, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, and Ac-D-Lys(DOTA)-D-Tyr-D-Lys(DOTA)-D-Lys(Tscg-Cys)-NH₂.

The kits may further comprise one or more radionuclides. Additionally or alternatively, in some embodiments of the kits of the present technology, the one or more radionuclides are selected from among ²¹³Bi, ²¹¹At, ²²⁵Ac, ¹⁵²Dy, ²¹²Bi, ²²³Ra, ²¹⁹Rn ²¹⁵Po, ²¹¹Bi, ²²¹Fr, ²¹⁷At, and ²⁵⁵Fm. Additionally or alternatively, in certain embodiments, the one or more radionuclides are selected from the group consisting of ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}OS, ¹⁹²Ir, ²⁰¹Tl, ²⁰³Pb, ⁶⁸Ga ²²⁷Th, and ⁶⁴Cu.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive GD2 protein in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more bispecific anti-GD2 SADA conjugates of the present technology capable of binding a GD2 protein in a biological sample; means for determining the amount of the GD2 protein in the sample; and means for comparing the amount of the immunoreactive GD2 protein in the sample with a standard. One or more of the anti-GD2 SADA conjugates may be labeled. The kit components, (e.g., reagents) can be packaged in a suitable container. The kit can further comprise instructions for using the kit to detect the immunoreactive GD2 protein.

The kit can also comprise, e.g., a buffering agent, a preservative or a protein-stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a GD2 protein in vitro or in vivo, or for PRIT-based treatment methods of GD2-associated cancers in a subject in need thereof. In certain embodiments, the use of the reagents can be according to the methods of the present technology.

### EXAMPLES

The present technology is further illustrated by the following Examples, which should not be construed as limiting in any way.

### Example 1: Materials and Methods

Study Design. To identify the effects of SADA domains on BsAbs used for multi-step drug-payload delivery, multiple SADA-BsAbs were expressed and characterized in vitro and in vivo, using both cell lines and patient-derived xenograft (PDX) models.

For in vivo experiments, sample sizes were determined based on the observed variation in tumor progression and response in previous studies (Cheal, S. M. et al., Eur J Nucl Med Mol Imaging 43, 925-937 (2016); Cheal, S. M. et al., Mol Cancer Ther 13, 1803-1812 (2014); Cheal, S. M. et al., J Nucl Med 58, 1735-1742 (2017); Cheal, S. M. et al., Theranostics 8, 5106-5125 (2018); Cheal, S. et al., Journal of Nuclear Medicine 59 (2018)). Mice were followed until tumors became too large (>1,500 mm³), and no data were excluded. All mice from the same treatment groups were co-housed in the same cage. Experiments using female mice were completely randomized after tumor implantation, but before their initial treatment. Experiments using male mice had cages randomized after tumor implantation and before the start of treatment. Blinding of treatment or experimental measurements was not carried out.

Animal Studies. Weights and tumor volumes were measured once per week, and overall mouse health was evaluated at least three times per week. Tumor volumes were calculated by caliper using the following formula: [(L)×(W)×(W)×0.5], where L is the longest diameter of the tumor, and W is the diameter perpendicular to L. Mice were sacrificed once tumor volumes reached 1.5-2.0 cm³. Throughout these experiments, treated mice did not display weight loss, hair loss or weakness outside of normal limits. Radiation studies were performed on female BALB/c nude mice (Envigo, Hsd:athymic Nude-Foxn1^{nu} 069(nu)/070(nu/+)) and both male and female BALB/c DKO mice (Taconic, C.Cg-Rag2^{tm1Fwa} Il2rg^{tm1Sug}/JicTac, 11503). Pharmacokinetic studies were carried out using female NOD.Cg-Prkdc^{scid} Il2rg^{tm1Wjl}/SzJ mice (NSG, Jackson Laboratory, 005557). Immunogenicity studies were performed on female C57BL/6J mice (Jackson Laboratory, 000664). Nude mice and C57BL6/J mice were purchased while DKO and NSG mice were bred in the MSKCC animal facility.

Nude mice were implanted subcutaneously with IMR32 neuroblastoma cells when they were 8-10 weeks old. After 16 days (tumors approximately 100-200 mm³), mice were treated intravenously with BsAb (1.25 nmol) and DOTA[¹⁷⁷Lu] (18.5 MBq) once per week each for up to 3-weeks (3×-3×). Alternative regimens treated mice once per week with BsAb and 3-times per week with DOTA[¹⁷⁷Lu], for either 1 week (lx-3×) or 2 weeks (2×-6×). External beam treated control mice were irradiated with 300 cGy of radiation.

DKO mice were implanted subcutaneously with digested neuroblastoma or small-cell lung cancer PDX tumors (each tumor was passaged into 10 new mice). Treatment began 18-20 days after implantation. Mice were treated intravenously with BsAb (1.25 nmol) and either Proteus[²²⁵Ac] (37 kBq) or DOTA[¹⁷⁷Lu] (55.5 MBq). For studies using Proteus[²²⁵Ac], mice were dosed once with BsAb and once with payload. For studies using DOTA[¹⁷⁷Lu], mice were dosed once per week with BsAb and payload, for 3 weeks. Differences in specific activity between the two Proteus[²²⁵Ac] preparations resulted in different molar doses despite equivalent activities (2.4 nmol vs 700 pmol). 3-step IgG-PRIT followed the same schedule as 2-step SADA-PRIT, with the additional clearing agent step occurring 4 hours prior to the administration of DOTA or Proteus payloads. 25 μg of DOTA-dendrimer clearing agent (Cheal et al., Bioconjug Chem. 31(3):501-506 (2020)) was used for all experiments where clearing agent was used. All cell line implantations used Matrigel (Corning, 354234) at a ratio of 3:1 by volume (Matrigel to cells). Plasma was collected retro-orbitally and stored at −80° C. until assayed. CBC measurements during treatment was done on freshly collected whole blood (EDTA-neutralized) using an HT5 Hematology Analyzer (Heska). Data was plotted using GraphPad Prism 8.

Pharmacokinetic Analysis. NSG mice were injected with 100 μg of P53-SADA-BsAb and bled serially over 7 days (30 minutes-168 hours). Blood was processed as plasma and frozen until all samples were acquired. Plasma concentrations of BsAb were determined by ELISA. Briefly, for each plate, half of the wells were coated with ganglioside GD2 overnight at 4° C. (EMD Millipore, 345743, 1 μg/ml in 90% ethanol, 20 μl/well), and half were left blank. Plates were washed with PBS and blocked with PBS supplemented with 0.5% bovine serum albumin (Sigma, A7906) for one hour at room temperature. Plasma samples were added at 1:100 and 1:200 dilutions (>48 hours) or 1:2000 and 1:4000 dilutions (0.5-24 hours) in duplicate across both coated and uncoated wells and incubated at 37° C. for 2.5 hours. P53-SADA-BsAb was used as a standard curve (100 ng/ml to 0.41 ng/ml, 3-fold dilutions). Samples were detected using a mouse anti-HIS specific secondary antibody (Biorad, clone AD1.1.10, MCA1396) for one hour at room temperature. Samples were then incubated with a rat anti-mouse detection antibody conjugated to horse-radish peroxidase (Jackson ImmunoResearch, 415-035-166) for one hour at 4° C. The color reaction was developed with o-phenylenediamine (Sigma, P8287-100TAB, 150 ul/well) and stopped with 5N sulfuric acid (30 ul/well). Plates were read at 490 nm using a Biotek H1 plate reader (Synergy) with the Gen5 software (version v2.09). Protein concentrations were calculated using a standard curve fitted to a linear regression. Pharmacokinetic analysis was carried out by non-compartmental analysis of the serum concentration-time data using WinNonlin software program (Pharsight Corp.).

Serum clearance measurements were also determined using ¹³¹I-labeled SADA-BsAb. SADA-BsAb were labeled with ¹³¹I (IBA Molecular or MSKCC) using precoated IODOGEN tubes (Pierce) as previously described for the radioiodination of the IgG-scFv-BsAb (Cheal, S. M. et al., Mol Cancer Ther 13, 1803-1812 (2014)). Purity of the ¹³¹I-SADA-BsAb was validated by SEC-HPLC. Each mouse (nude, tumor free) was injected with 740 kBq of ¹³¹I-SADA-BsAb and bled serially (0.5 to 48 hours). Blood samples were radio-assayed on a gamma counter (PerkinElmer, Wallac Wizard 3 automatic gamma counter) and plotted using GraphPad Prism 8.

Immunogenicity Analysis. C57BL/6J mice were injected with P53-SADA-BsAb or IgG-scFv-BsAb (0.5 nmol) on days 0 and 28, intravenously and intraperitoneally, respectively. Mice were bled retro-orbitally on days 27 and 55. Blood was processed as plasma and frozen at −80° C. until all samples were acquired. Plasma concentrations of each BsAb were determined by ELISA. Briefly, for each plate, half of the wells were coated with P53-SADA-BsAb or IgG-scFv-BsAb (10 μg/ml in PBS, 50 μl/well) overnight at 4° C., and the other half were left blank). After this, plates were washed with PBS and blocked with PBS supplemented with 0.5% bovine serum albumin (Sigma, A7906) for one hour at room temperature. Plasma samples were added at 1:100 and 1:200 dilutions in duplicate across both coated and uncoated wells and incubated at 37° C. for 2.5 hours. A standard curve was generated using either mouse-anti-HIS antibody (SADA-BsAb) or an anti-human IgG-hinge (IgG-scFv-BsAb, Southern Biotech, Clone 4E3, 9052-01) monoclonal antibodies. Next, samples were detected with a goat anti-mouse antibody detection antibody conjugated to horse-radish peroxidase (Jackson ImmunoResearch, 115-005-003). The color reaction was developed with o-phenylenediamine (Sigma, P8287-100TAB, 150 ul/well) and stopped with 5 N sulfuric acid (30 ul/well). Plates were read at 490 nm using a Biotek H1 plate reader (Synergy) with the Gen5 software (version v2.09). Protein concentrations were estimated using a standard curve fitted to a linear regression. Data was plotted using GraphPad Prism 8.

Anatomic and Clinical Pathology for Toxicology Assessment. Mice were sacrificed by carbon dioxide asphyxiation, and immediately dissected and fixed in 10% neutral buffered formalin. Age-matched littermates were used as reference in all studies. Tissues were processed in ethanol and xylene and embedded in paraffin in a Leica ASP6025 tissue processor. Paraffin blocks were sectioned at 5 microns, stained with hematoxylin and eosin (H&E), and histopathologic examination was performed by two board-certified veterinary pathologists. (SM, AOM). The following tissues were processed and evaluated: heart, lungs, thymus, kidneys, liver, gallbladder, stomach, duodenum, jejunum, ileum, cecum, colon, mesenteric lymph node, salivary glands, submandibular lymph node, uterus, cervix, vagina, urinary bladder, spleen, pancreas, adrenals, ovaries, oviducts, trachea, esophagus, thyroid, parathyroid, skin (trunk, perigenital, head), mammary glands, bones (femur, tibia, sternum, vertebrae, skull), bone marrow (femur, tibia, sternum, vertebrae), stifle joint, skeletal muscles (hind limb, spine), nerves (hind limb, spine), spinal cord, oral cavity, teeth, nasal cavity, eyes, harderian gland, pituitary, brain, ears. For serum chemistry, blood was collected into tubes containing a serum separator and centrifuged. Serum samples were analyzed on an AU 680 chemistry analyzer (Beckman Coulter Inc, Pasadena, Calif.) and the concentration of the following analytes was determined: alkaline phosphatase, alanine aminotransferase, aspartate aminotransferase, creatine kinase, gamma-glutamyl transpeptidase, albumin, total protein, globulin, total bilirubin, blood urea nitrogen, creatinine, cholesterol, triglycerides, glucose, calcium, phosphorus, chloride, potassium, and sodium. Na/K ratio, albumin/globulin ratio were calculated. For hematology, blood was collected into tubes containing EDTA and automated Complete blood counts (CBC) were performed on a Procyte Dx (Idexx laboratories Inc., Westbrook, Me.) with manual differential performed by blood smear examination for validation. For further kidney analysis, sections of kidney were also stained with the TdT-mediated dUTP-biotin nick end labeling (TUNEL) method as previously described (Gavrieli, Y. et al., J Cell Biol 119, 493-501 (1992)) and IHC against cleaved caspase 3 (Cell Signaling Technology Inc., Cat #9661) were performed on a Leica Bond RX automated stainer using Bond reagents (Leica Biosystems, Buffalo Grove, Ill.). Following heat-induced epitope retrieval in a citrate buffer, the primary antibody was applied at a 1:250 concentration and was followed by a polymer detection system (DS9800, Novocastra Bond Polymer Refine Detection, Leica Biosystems). The chromogen was 3,3 diaminobenzidine tetrachloride (DAB), and sections were counterstained with hematoxylin. The total number of TUNEL positive and CC-3 immunoreactive cells were counted in ten, 400×fields on an Olympus BX45 microscope with a UPlanFL 40×/0.75 objective (Olympus Corp., Tokyo, Japan).

PET/CT Imaging Analysis. Female nude mice were implanted with subcutaneous IMR32 neuroblastoma xenografts on day 0. On day 16, mice were administered BsAb (1.25 nmol) intravenously. On day 18, mice were administered DOTA[⁸⁶Y] (3.7 MBq, 30 pmol). On day 19 mice were imaged using PET/CT (Siemens, Inveon PET/CT scanner) for a minimum of 1×10⁶ coincidence events while under the influence of 1.5-2% isofluorane (Baxter Healthcare). Typically PET data were collected for 30 minutes followed by CT. Whole-body CT scans were acquired with a voltage of 80 kV and 500 μA. A total of 120 rotational steps for a total of 220° were acquired with a total scan time of 120 s and 145 ms per frame exposure. List-mode emission data were sorted into two-dimensional histograms by Fourier rebinning, and the images were reconstructed using a 2DOSEM algorithm (16 subsets, four iterations) into a 128×128×159 (0.78×0.78×0.80 mm) matrix. The image data were normalized to correct for nonuniformity of response of the PET, dead-time count losses, positron branching ratio, and physical decay to the time of injection but no attenuation, scatter, or partial-volume averaging correction was applied. 3 mice were imaged together and separated during analysis.

Tissue Biodistribution Analysis. Female nude mice were implanted with subcutaneous IMR32 neuroblastoma xenografts on day 0. On day 16, mice were administered BsAb (1.25 nmol) intravenously. On day 18, mice were administered DOTA[¹⁷⁷Lu] (1.85 to 18.5 MBq, 10 to 100 pmol). Mice were sacrificed and dissected 2 hours, 24 hours, 48 hours, 72 hours or 120 hours after administration of DOTA[¹⁷⁷Lu]. The following tissues were collected and residual radiation was counted on a gamma scintillation counter (PerkinElmer, Wallac Wizard 3 automatic gamma counter): Blood, brain, spine, tumor, heart, lungs, liver, spleen, stomach, small intestine, large intestine, kidneys, muscle, long bone, and tail.

Tissue Dosimetry Analysis. Dosimetry estimates were modeled using tissue biodistribution results from each BsAb. For each tissue, the non-decay-corrected time-activity concentration data were fit using Excel to a 1-component, 2-component, or more complex exponential function as appropriate, and analytically integrated to yield the accumulated activity concentration per administered activity (MBq-h/g per MBq). The ¹⁷⁷Lu equilibrium dose constant for non-penetrating radiations (8.49 g-cGy/MBq-h) was used to estimate the tumor-to-tumor and select organ-to-organ self-absorbed doses, assuming complete local absorption of the ¹⁷⁷Lu beta rays only and ignoring the gamma ray and non-self-dose contributions.

Protein Sequences. Anti-GD2 antibodies used V_{H} and V_{L} domains from hu3F8 (Cheung et al., Oncoimmunology 1, 477-486 (2012)). Anti-DOTA antibodies used V_{H} and V_{L} domains from huC825. SADA domains were derived from select portions of TP53, TP63, TP73, or HNRPC or SNAP23 genes. The IgG-scFv-BsAb proteins used a human IgG1 framework that contained both N297A and K322A mutations to eliminate Fc receptor and complement binding activities, respectively. All scFv domains included six G₄Si domains (SEQ ID NO: 19) between the V_{H} and V_{L} domains, the SADA-BsAb included four additional G₄S₁ domains (SEQ ID NO: 20) between both scFv, and the IgG-scFv-BsAb included three additional G₄S₁ domains (SEQ ID NO: 21) between the C_{L} and scFv domains.

Protein Production. All SADA-BsAb proteins were expressed using the Expi293 Expression System (Invitrogen, A14524), according to manufacturer's instructions. Briefly, expression plasmids for each bispecific antibody (BsAb) were amplified and purified using the PureLink™ HiPure Plasmid Filter Maxiprep Kit (Invitrogen, K210016), then diluted and incubated with Expifectamine (Invitrogen) for 20 minutes before being added to cell suspensions. IgG-scFv-BsAb proteins were expressed using previously developed stable expression cell lines (CHO—S) (Cheal, S. M. et al., Mol Cancer Ther 13, 1803-1812 (2014)). In both cases cells were incubated in shaker culture until cell viability dropped <70% (4-14 days). IgG-based proteins were purified with a protein A column using a P920 AKTA FPLC (GE) and eluted with a 1:1 (v/v) mix of citric acid buffer [43 mM citric acid (Sigma A104) 3 mM sodium citrate (Sigma, S1804)] and sodium citrate solution [25 mM sodium citrate (Sigma), 150 mM sodium chloride (Fisher, S271)]. SADA-BsAb proteins were purified using prepacked Ni²⁺ NTA columns (GE, 11003399) and eluted using 250 mM imidazole (Sigma, 792527). All proteins were buffer exchanged overnight into a pH 8.2 sodium citrate solution [25 mM sodium citrate (Sigma), 150 mM sodium chloride (Fisher)] and subsequently analyzed by SEC-HPLC (Shimadzu) to determine purity.

Radiometal Labeling. For DOTA[⁸⁶Y], S-2-(4-Aminobenzyl)-1,4,7,10-tetraazacyclododecane tetraacetic acid (DOTA, Macrocyclics, B-200, 181065-46-3) was mixed with ⁸⁶Y nitrate (Radiological Chemistry and Imaging Laboratory at Washington University in St. Louis) 60 minutes at 80° C. Labeled DOTA was separated from free radiometal by passing through a sepak column (Waters). For DOTA[¹⁷⁷Lu], DOTA was incubated with ¹⁷⁷LuCl₃ (Perkin Elmer) at a ratio of 37 MBq to 1.1 mmol of DOTA in ammonium acetate (pH 5.6) for 60 minutes at 85° C. For Proteus[²²⁵Ac], ²²⁵Ac nitrate (Oak Ridge National Laboratory) was mixed with Proteus for 30 min at 60° C. After incubation the sample was purified using a Sephadex C-25 column (GE) pre-equilibrated with 6 mL of normal sterile isotonic saline solution (NSS).

Cells and Cell Lines. IMR32 cell lines were obtained from ATCC (Manassas, Va.). IMR32 cells were transfected with luciferase before use in all assays. M14 cell lines were obtained from University of California, Los Angeles, and transfected with luciferase before use in all assays. IMR32 and M14 melanoma cells were validated by STR. All cell lines were maintained in RPMI medium (Corning, 15-040-CM) supplemented with 10% heat inactivated fetal calf serum (VWR, 96068-085), 2 mM L-glutamine (Sigma, G5792), and 1% penicillin/streptomycin (Corning, 30-002-CI). Neuroblastoma patient-derived xenograft (PDX) tumors were established from surgical samples of patients (consented in protocol NCT00588068).

CellBinding Measurements. Cell binding of BsAbs were measured by flow cytometry. M14 melanoma cells were incubated with each BsAb and detected using both a biotinylated-DOTA[¹⁷⁵Lu] and a PE-conjugated streptavidin protein (Sigma, S3402-1ML). Biotinylated-DOTA[¹⁷⁵Lu] was generated at the Organic Synthesis Core at MSKCC. All incubations were for 30 minutes at 4° C. Experiments were repeated multiple times, with graphs presenting a single representative experiment. Samples were acquired using a BD FACSCalibur and analyzed by FlowJo 10.5.3 and GraphPad Prism 8.

Affinity Measurements. Binding kinetics were evaluated using SPR (GE, Biacore T200) as described previously (Santich, B. H. et al., Sci Transl Med 12, (2020)). Briefly, SA chips were coated with biotin-GD2 (Elicityl Oligotech) A five-step titration series of each BsAb was flowed over them, followed by two blank cycles and two regeneration cycles. Binding affinities were calculated using a two-state reaction model with the GE Biacore Evaluation software. Data were plotted using GraphPad Prism 8.

Statistical Analyses. All statistical analyses were performed using Prism software version 8.4 (GraphPad). Statistical significances were determined by Man Whitney tests (ADA titers), two-way analysis of variance (ANOVA) with subsequent Tukey or Sidak correction (tumor responses), or a Log-rank (Mantel-Cox) test (survival analyses). For all statistical tests, a P value of <0.05 was used to denote statistical significance. All error bars denote the standard deviation, unless otherwise noted in the figure legends.

### Example 2: TP53 and TP63 can Stably Tetramerize anti-GD2×anti-DOTA BsAb

Anti-GD2/anti-DOTA SADA-BsAb conjugates were designed by fusing a small tetramerizing SADA domain to a humanized tandem single-chain fragment (scFv) BsAb, where one scFv bound to tumor antigen ganglioside GD2 and the other bound to DOTA, a small molecule payload that chelates lutetium. The resulting SADA-BsAb would have a self-assembled size of ˜200 kDa and a disassembled size of ˜50 kDa (FIG. 1B). Candidate SADA domains were selected based on several criteria: human derived, non-membrane protein, naturally tetramerizing, and below 15 kDa in molecular size. Six candidate sequences were identified including TP53, TP63 and TP73 (FIG. 14). Among them, four expressed sufficiently well as a SADA-BsAb (>1 mg/L) and demonstrated high purity at the expected tetrameric sizes (FIG. 1C, FIG. 15). Of these four sequences, those derived from TP53 and TP63 were chosen based on their superior stability at 37° C., high expression yield, and high purity.

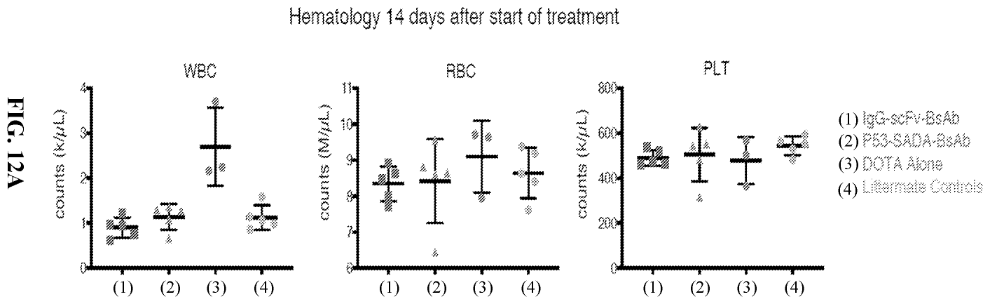

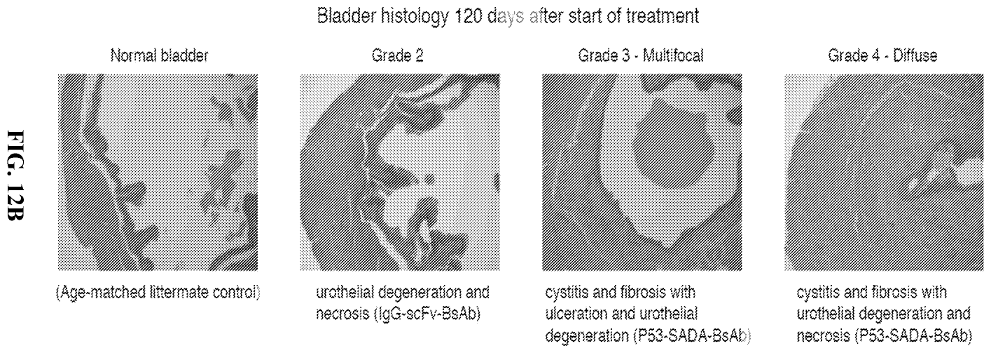

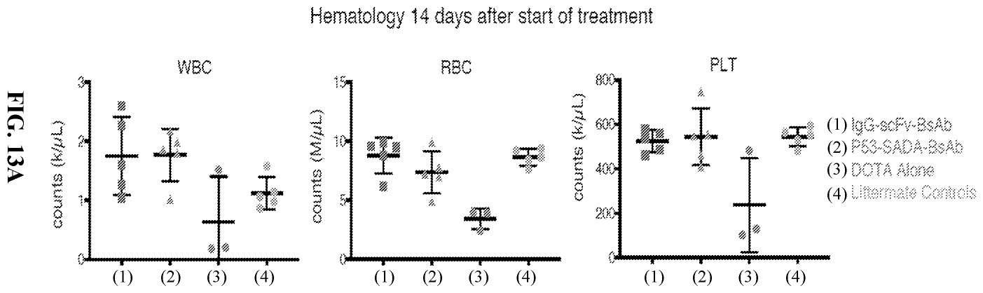

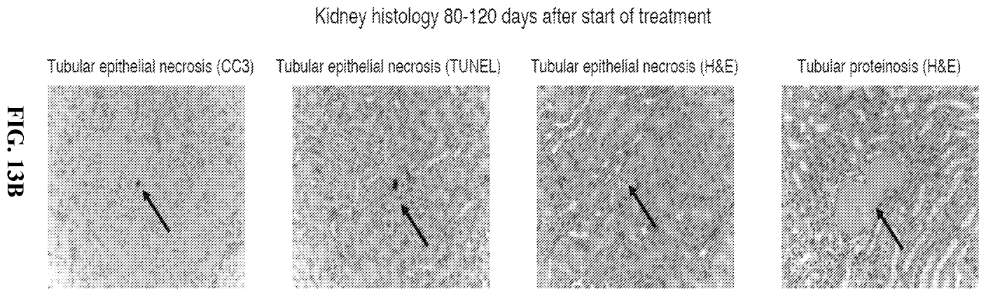

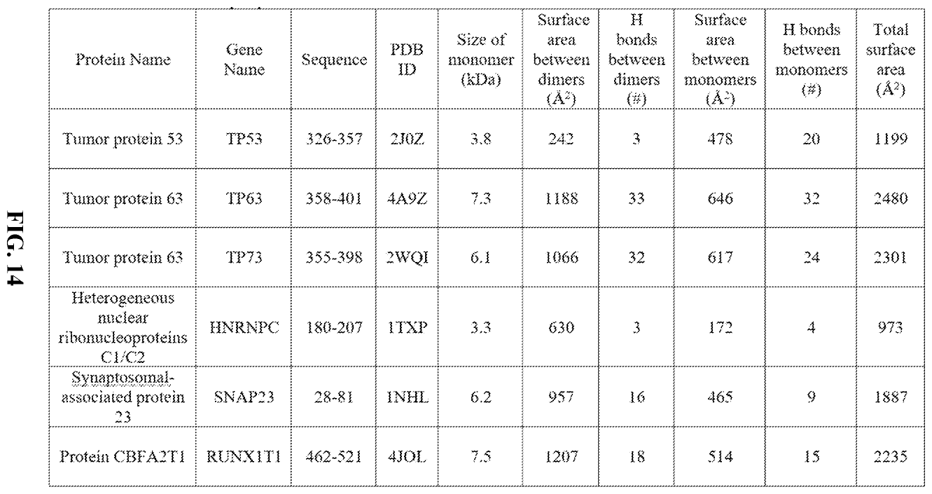

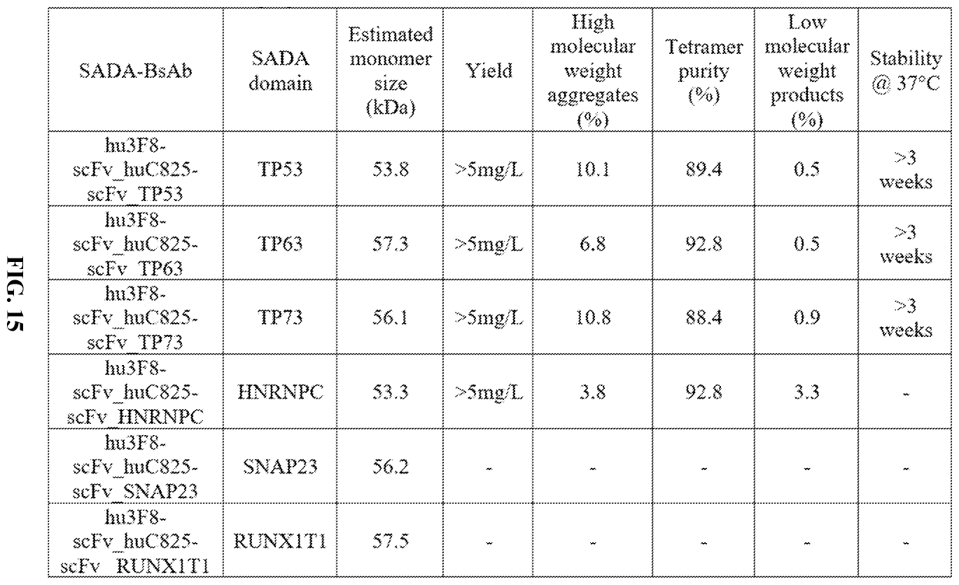

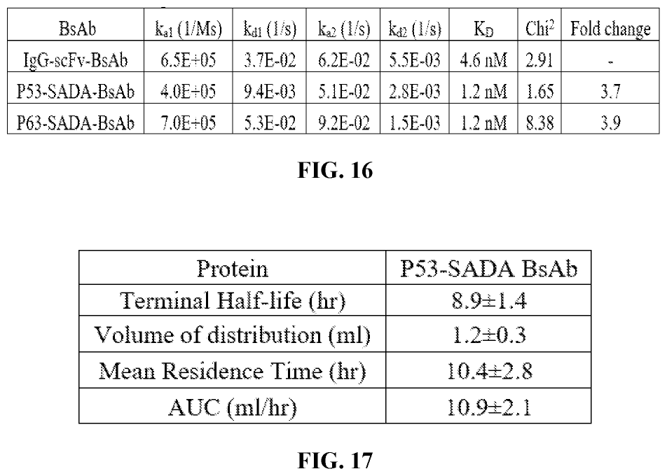

The binding affinity of P53-SADA-BsAb and P63-SADA-BsAb were evaluated by surface plasmon resonance (SPR) and flow cytometric analysis (FIGS. 1D and 1E, FIG. 16). SPR revealed enhanced GD2 binding avidity (K_{D}) and slower dissociation (k_{off}) for both P53- and P63-SADA-BsAb compared to the corresponding anti-GD2×anti-DOTA IgG-[L]-scFv formatted BsAb (Cheal, S. M. et al., Mol Cancer Ther 13, 1803-1812 (2014)) used in 3-step IgG-PRIT (1.2 nM vs 4.6 nM K_{D}, respectively). In addition, flow cytometry confirmed P53- and P63-SADA-BsAb could bind DOTA payloads (biotinylated-DOTA[¹⁷⁵Lu]) to tumor cells (GD2⁺ neuroblastoma), with comparable binding intensity to the IgG-scFv-BsAb.

### Example 3: SADA-BsAbs Rapidly Clear from the Body without Compromising Tumor Uptake

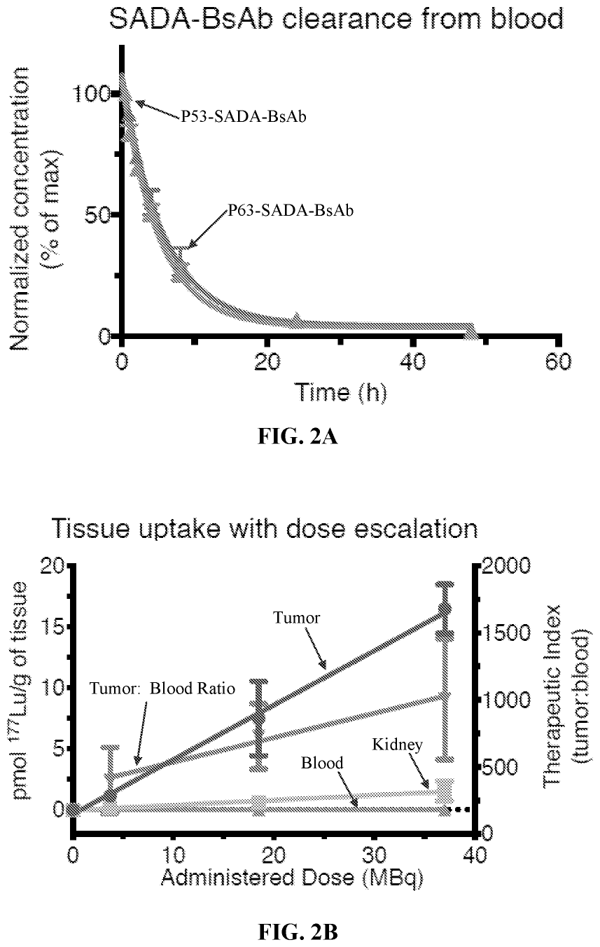

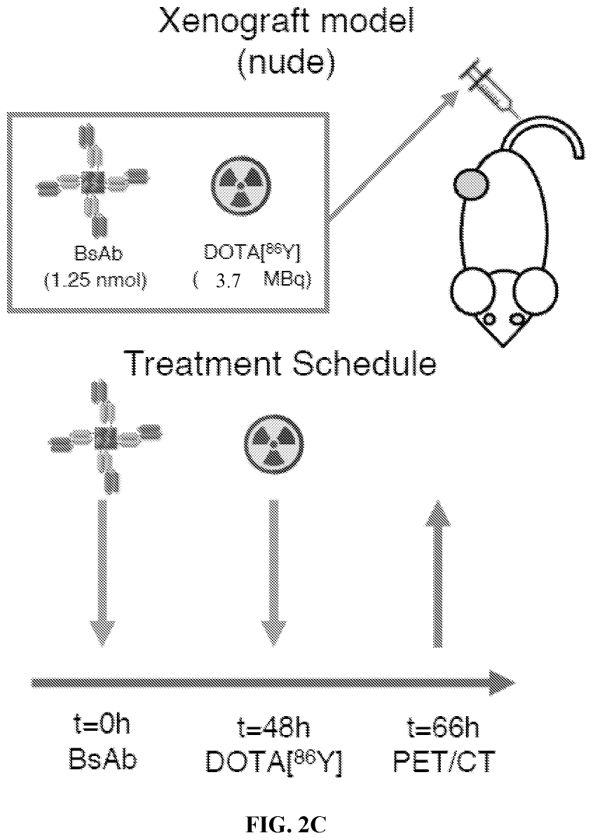

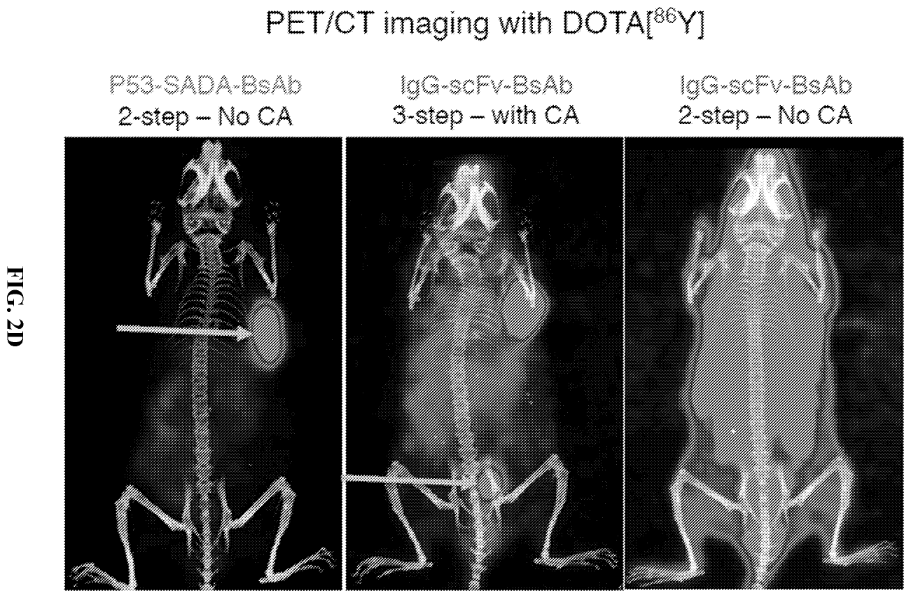

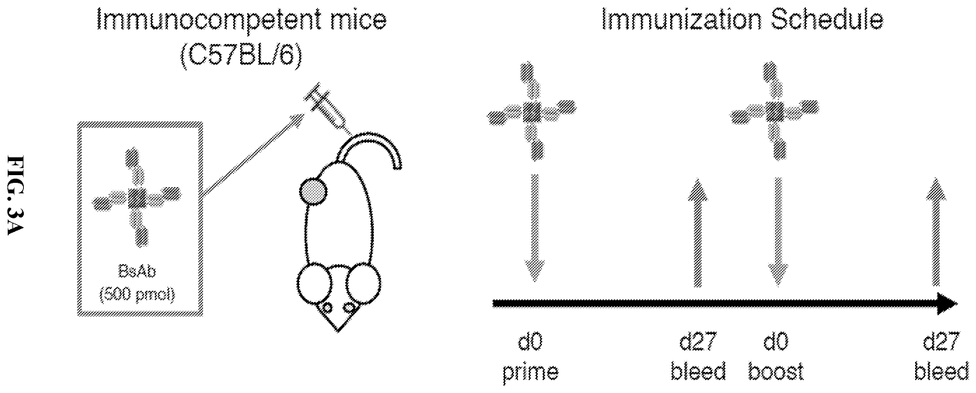

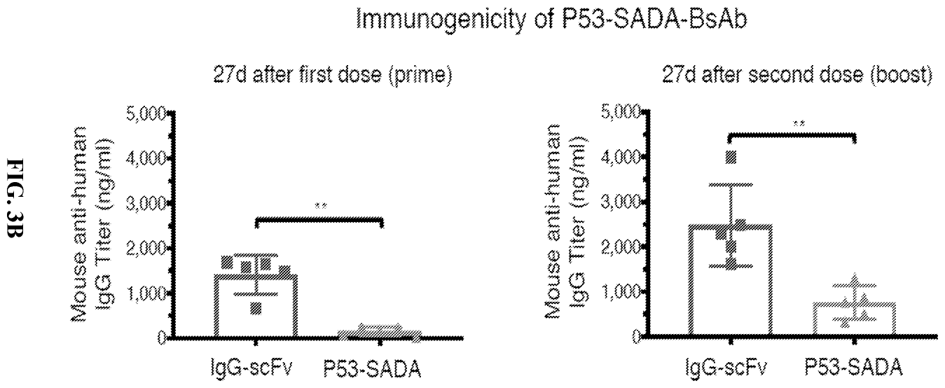

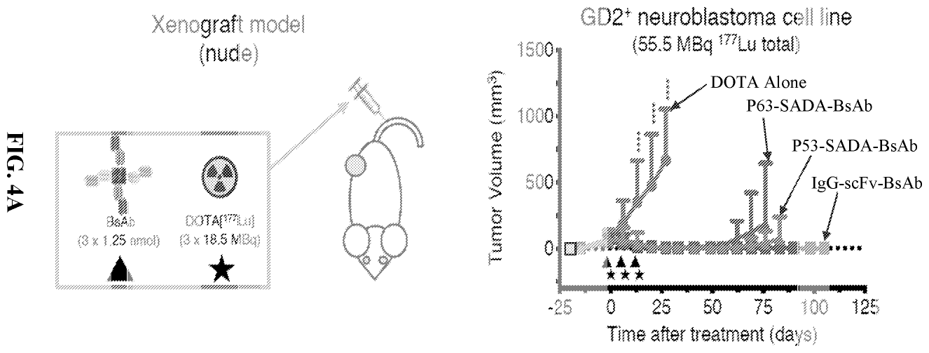

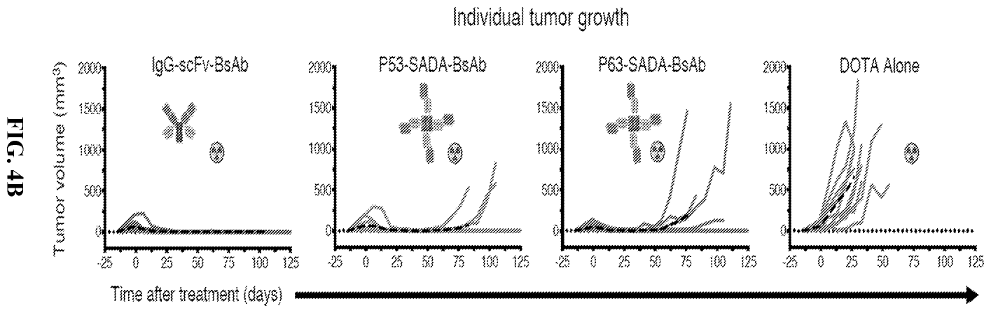

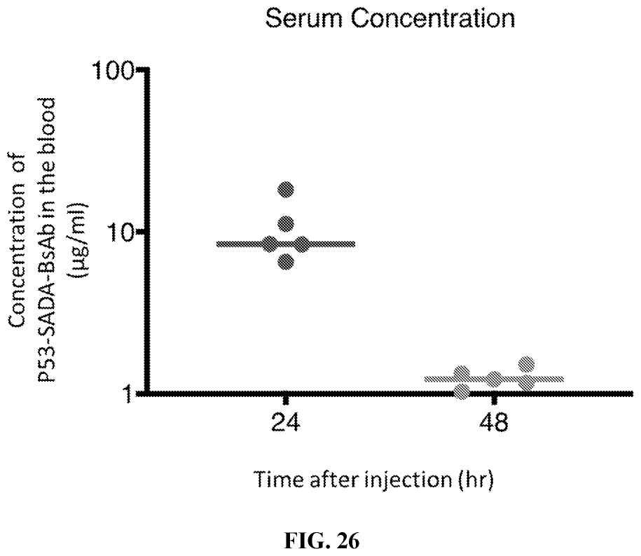

Previous studies have shown that monomeric or dimeric anti-GD2 tandem-scFv BsAb exhibit very short terminal half-lives (t_{1/2}=0.5 hour) in mice (Ahmed et al., Oncoimmunology 4, e989776 (2015)), while anti-GD2 IgG or IgG-scFv-BsAb are much longer (t_{1/2}=72 hours) (Santich, B. H. et al., Sci Transl Med 12 (2020)). In contrast, both P53- and P63-SADA domains substantially altered the pharmacokinetics of the BsAb monomers (t_{1/2}=9 hours) while also permitting their complete removal from the blood within 48 hours (FIG. 2A, FIG. 17). As shown in FIG. 26, P53-SADA-BsAb levels were still detectable in blood 24 hours post administration, and were substantially cleared from blood after 48 hours.

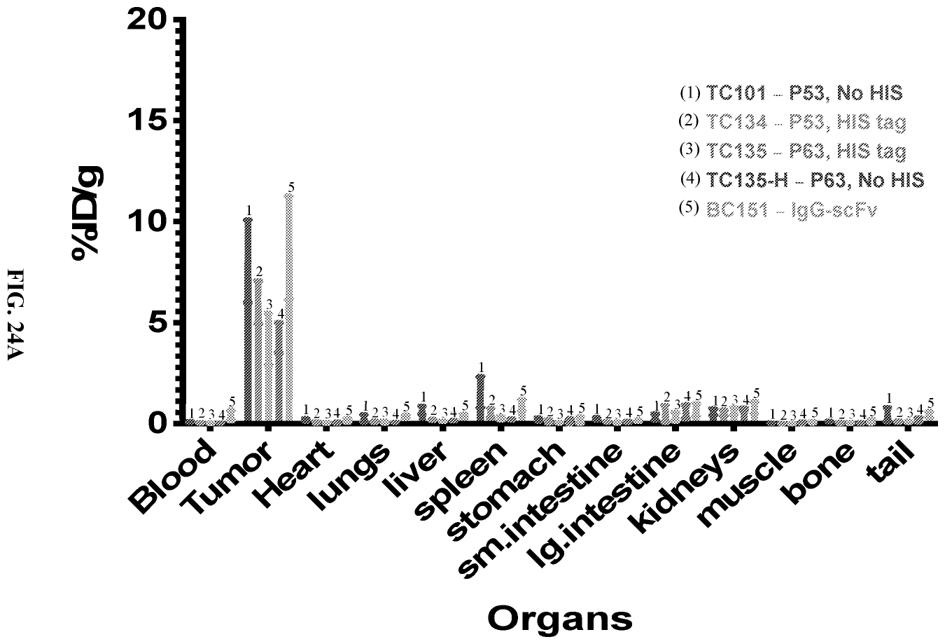

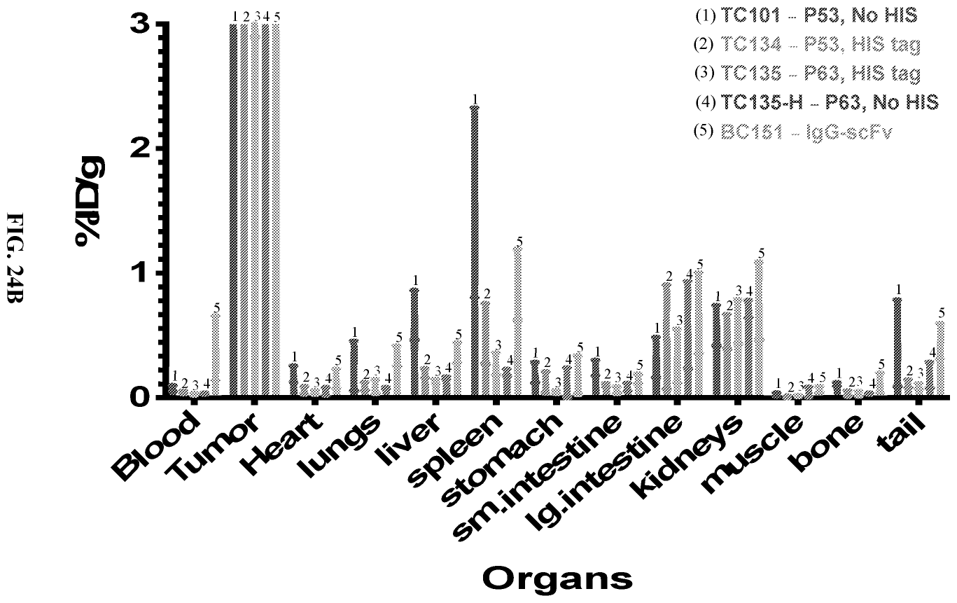

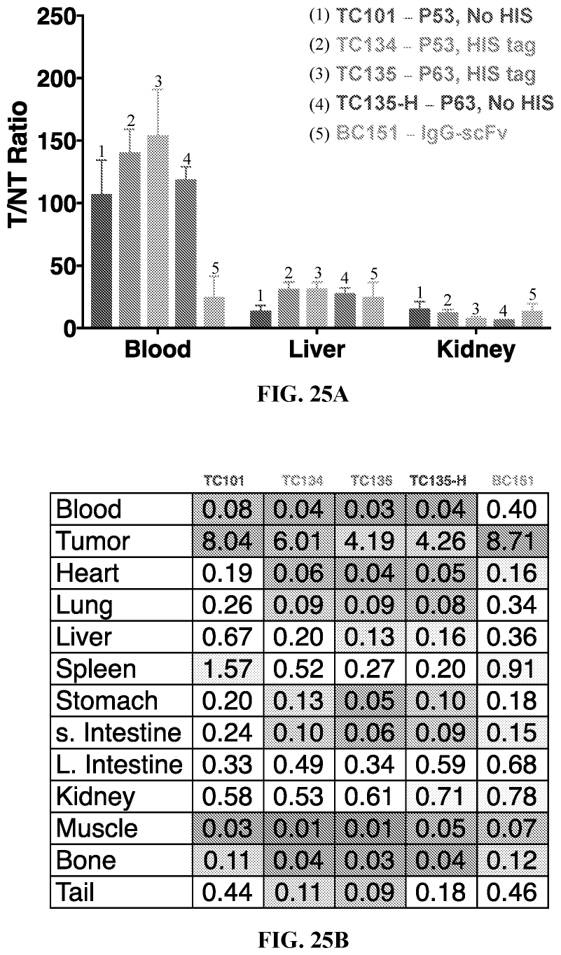

To measure the utility of SADA on payload delivery, a multi-step targeting strategy using DOTA[¹⁷⁷Lu] as the cytotoxic payload was employed. The protocol began with a dose escalation study in athymic nude mice bearing subcutaneous GD2⁺ neuroblastoma xenografts (IMR32). Mice were dosed with P53-SADA-BsAb (1.25 nmol), followed 48 hours later with 3.7, 18.5 or 37 MBq of DOTA[¹⁷⁷Lu] (20, 100, or 200 pmol, respectively). Tumor uptake of the payload revealed a strong linear correlation with administered dose (slope of 0.45 pmol/g/MBq, R²=0.94), while activity in the blood remained low at all dose levels (slope <0.001, R²=0.70), resulting in higher tumor to blood ratios with higher doses of payload (FIG. 2B, Pearson coefficient of 0.9939). Kidney uptake also increased with administered dose but at a much shallower slope (slope=0.04, R²=0.77). These results contrasted to previous studies of 3-step IgG-PRIT (Cheal, S. M. et al., Eur J Nucl Med Mol Imaging 43, 925-937 (2016)), where tumor uptake plateaued at about 11 pmol/g, suggesting that SADA-BsAb could more effectively deliver DOTA payloads to the tumor, with minimal exposure to the kidneys or blood. FIGS. 24A-24B and FIGS. 25A-25B show that kidney uptake was not impacted by the presence or absence of a 6×HIS tag (SEQ ID NO: 41) in the SADA-BsAbs.

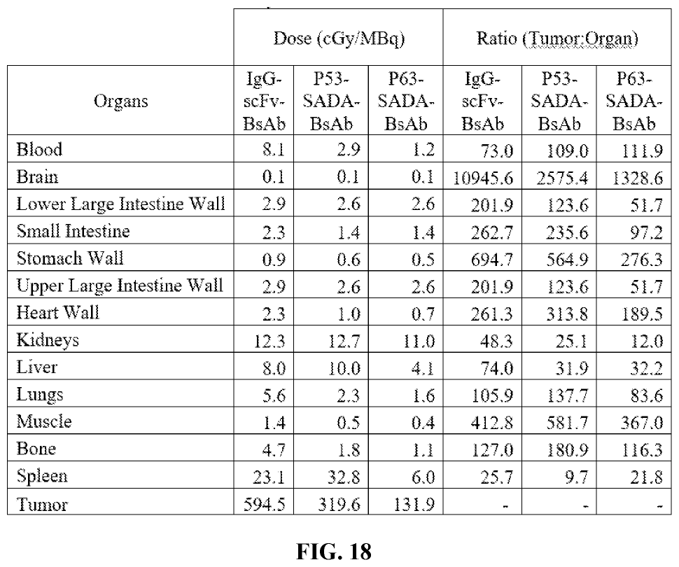

Payload dosimetry estimates for 2-step SADA-PRIT were generated from serial biodistribution studies using the same model (FIG. 18). Here, both P53- and P63-SADA-BsAb were administered without clearing agent, while IgG-scFv-BsAb followed the 3-step regimen (with clearing agent). While P53-SADA-BsAb and the IgG-scFv-BsAb delivered comparable total doses of radiation to the tumor and kidneys, both P53- and P63-SADA-BsAb delivered substantially lower doses to the blood (1.2-2.9 cGy/MBq vs 8.1, TI>100:1) and bone marrow (1.1-1.8 cGy/MBq vs 4.7, TI>120:1) compared to the IgG-scFv-BsAb. From these estimates, P53-SADA-BsAb was expected to safely deliver an absorbed dose of 5,000 cGy to the tumor from a 15 MBq (405 μCi) dose of DOTA[¹⁷⁷Lu] payload, with kidneys and blood receiving only 191 cGy and 44 cGy, respectively.

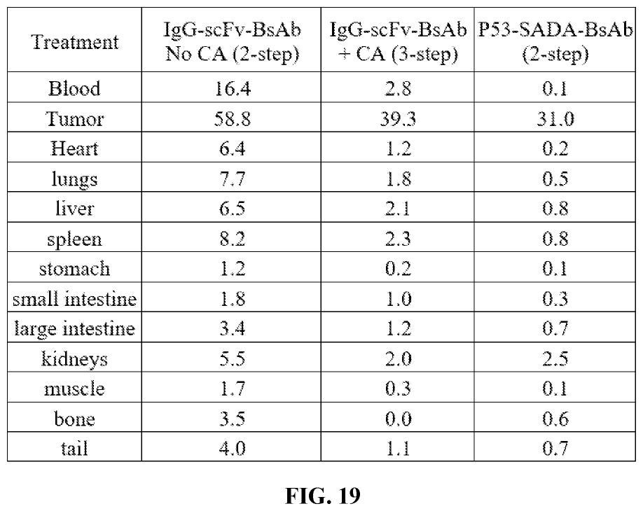

Since DOTA payloads can used for both therapeutic and diagnostic (theranostic) applications, the quantitative payload delivery of P53-SADA-BsAb using positron emission tomography (PET) was evaluated by swapping ¹⁷⁷Lu with ⁸⁶Y (FIGS. 2C-2D). Xenografted nude mice were dosed with P53-SADA-BsAb at t=0, followed by DOTA[⁸⁶Y] at t=48 h, and were imaged by PET/CT at t=66 h (FIG. 19). For comparison, two additional groups were included: mice dosed with (i) IgG-scFv-BsAb and DOTA[⁸⁶Y] without clearing agent (2-step) or (ii) IgG-scFv-BsAb and DOTA[⁸⁶Y] with clearing agent (3-step). IgG-scFv-BsAb administered without clearing agent resulted in significant retention of DOTA[⁸⁶Y] payload in the blood due to the high amounts of residual circulating BsAb. By contrast, inclusion of clearing agent after administering the IgG-scFv-BsAb improved tumor contrast but also increased gut uptake, possibly due to the hepatobiliary clearance. Treatment with P53-SADA-BsAb (without clearing agent), however, gave the best contrast, displaying strong tumor uptake and almost no detectable signal in any other organ.

These results confirmed that the SADA-BsAbs of the present technology can effectively target payloads to the tumor. Accordingly, the anti-GD2 SADA conjugates of the present technology are useful in both therapeutic and diagnostic (theranostic) applications.

### Example 4: P53-SADA-BsAb is Significantly Less Immunogenic than IgG-scFv-BsAb

To test whether SADA-BsAbs exhibited less immunogenicity, immunocompetent mice were immunized (day 0) and challenged (day 28) with P53-SADA-BsAb or IgG-scFv-BsAb, and anti-drug antibody (ADA) titers were measured in the plasma (FIGS. 3A-3B). Despite their high sequence homology, mice immunized with P53-SADA-BsAb showed significantly lower ADA titers than mice immunized with IgG-scFv-BsAb after both primary and secondary immunizations (P=0.008). These results demonstrate that SADA-BsAbs are less immunogenic compared to IgG-scFv-BsAbs, with respect to the emergence of ADA that is typically seen in IgG-based therapies. Such a benefit is critical to clinical translation, where multiple doses of antibody might be needed.

### Example 5: SADA-BsAbs Safely Deliver Beta-Emitter Payloads to Ablate Established Neuroblastoma Tumors

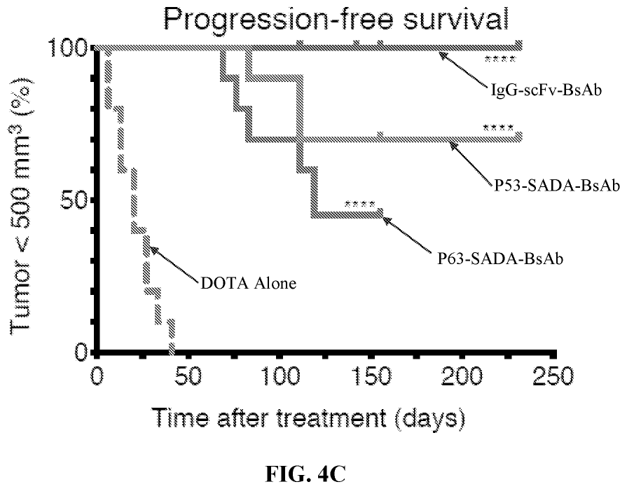

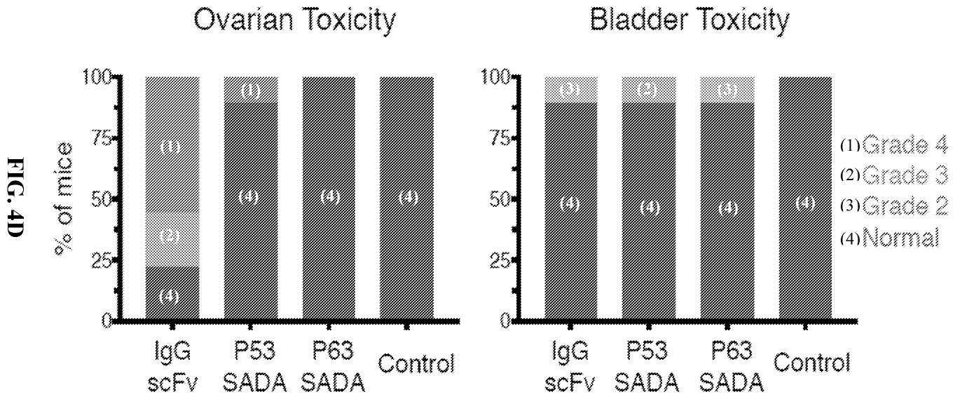

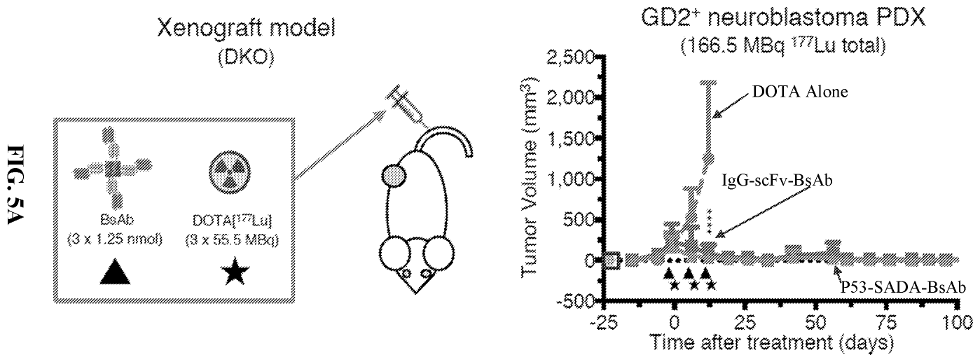

The anti-tumor function of 2-step SADA-PRIT was evaluated using the same xenograft model as before (FIG. 4A). Mice were treated using a 3×-3×schedule, where each week, for three weeks, one dose of BsAb (1.25 nmol) was followed by one dose of DOTA[m¹⁷⁷Lu]48 hours later (18.5 MBq, 100 pmol). Within two weeks all treated tumors had decreased in size, and within five weeks they had completely responded (FIGS. 4A-4C), extending survival significantly (median survival >115 d vs 20 d for control groups, P<0.0001). After extended follow-up, 100% of mice treated with IgG-scFv-BsAb (10/10) remained in complete remission, compared to 70% (7/10) for P53-SADA-BsAb and 50% (5/10) for P63-SADA-BsAb. The complete remission rates observed with the SADA-BsAb 3×-3×schedule were significantly improved compared to the 0% complete remission rate observed in a previous experiment in which mice with significant tumor burden (>500 mm³ tumor volumes) received a single 250 μg (1.25 nmol) dose of P53-SADA-BsAb (lacking HIS tag) followed by 2 mCi of ¹⁷⁷Lu-Bn-DOTA 24 hours later.

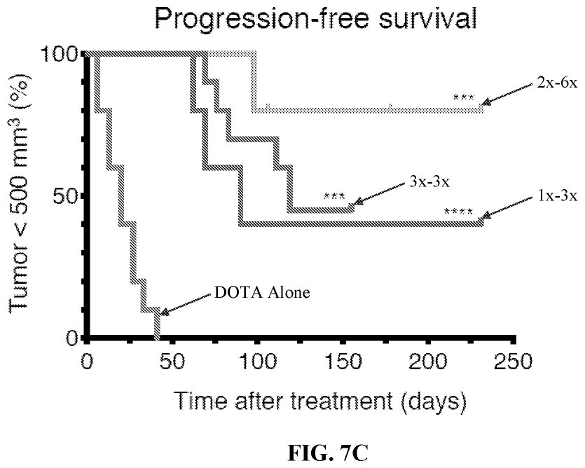

Two additional treatment schedules were explored for 2-step SADA-PRIT, where each dose of P63-SADA-BsAb was followed by three doses of DOTA[¹⁷⁷Lu] instead of one (FIGS. 7A-7D), either for one week (lx-3×, 55.5 MBq of DOTA[¹⁷⁷Lu] per mouse) or two weeks (2×-6×, 111 MBq of DOTA[1⁷Lu] per mouse). The first dose of DOTA[¹⁷⁷Lu] was administered 48 hrs after being administered the SADA-BsAb, the second dose of DOTA[¹⁷⁷Lu] was administered 24 hours after the first dose of DOTA[¹⁷⁷Lu], and the third dose of DOTA[¹⁷⁷Lu] was administered 24 hours after the second dose of DOTA[¹⁷⁷Lu]. While all treated tumors completely responded, the 2×-6×schedule displayed the best durability (median survival >250 d for 2×-6×vs 119 d for 1×-3×), which suggested higher doses of payload could improve response durability. Complete remission rates were as follows: lx-3×: 60% remission vs. 2×-6×: 20% remission.

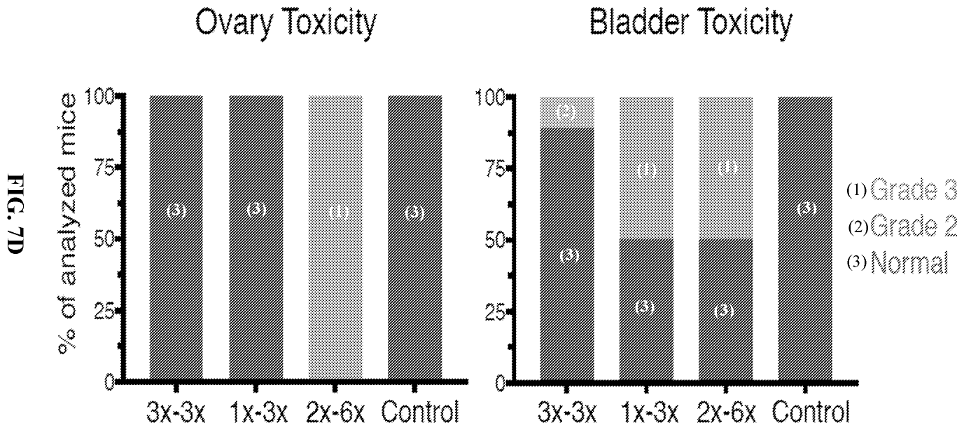

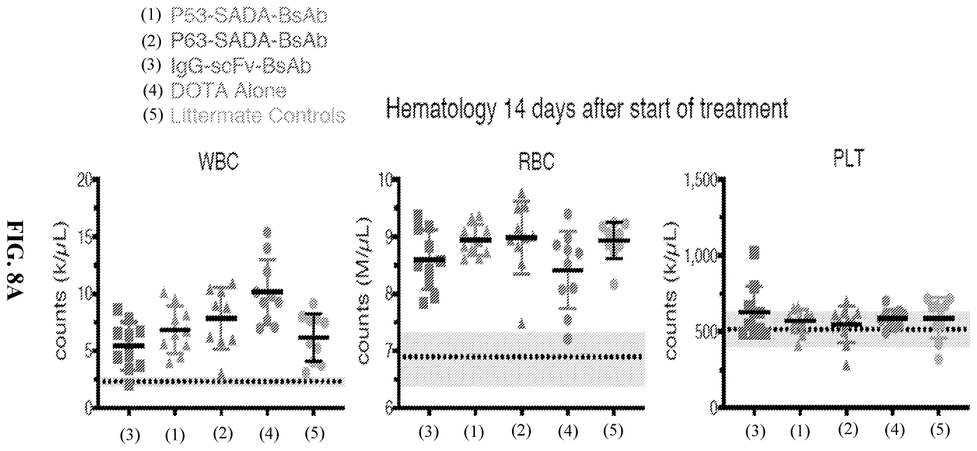

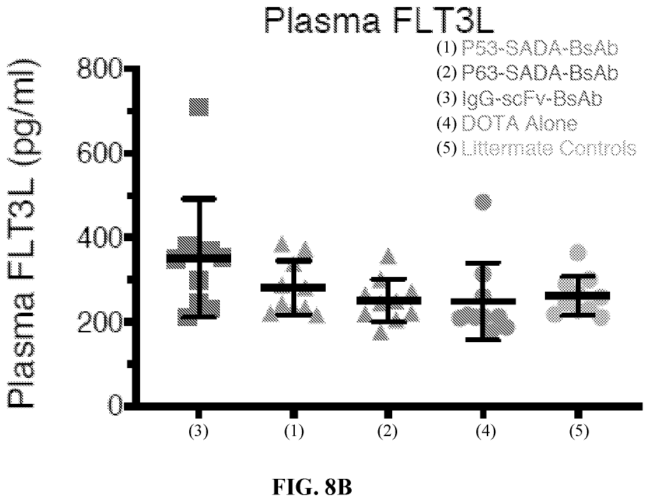

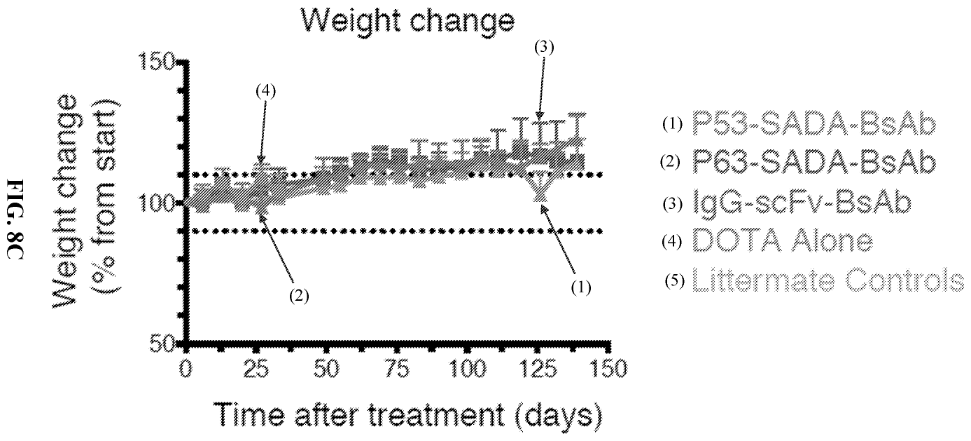

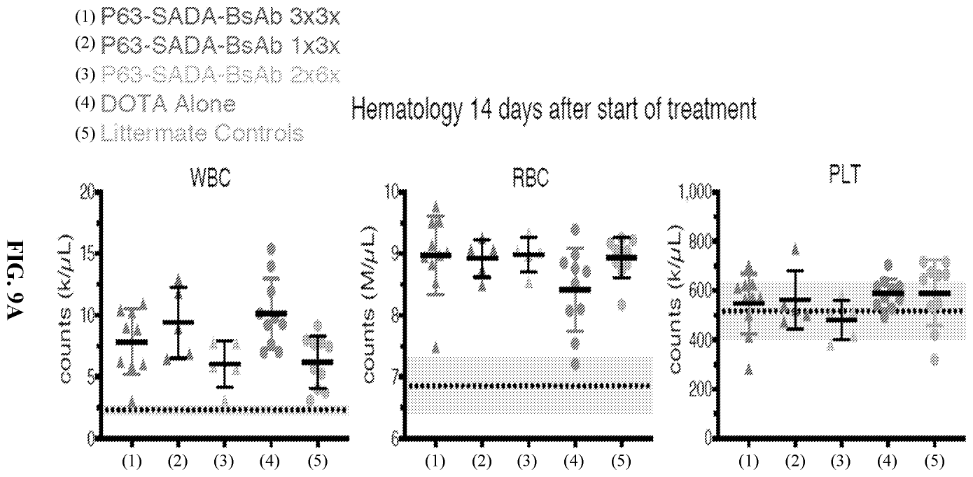

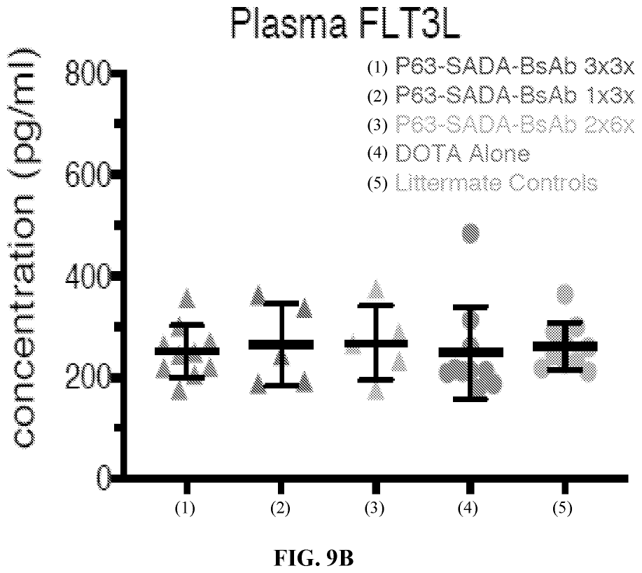

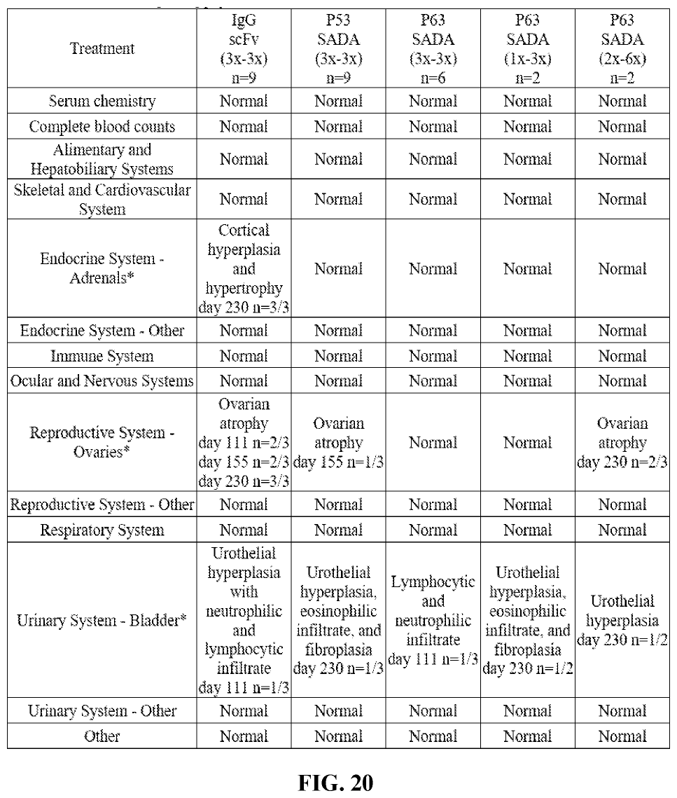

Treatment-related toxicities stemming from SADA-BsAbs were determined by in-life observation (body weight), clinical pathology (complete blood counts, serum chemistry, plasma FLT3L cytokine) and anatomic pathology (gross necropsy and histopathology) after both short-term (0-30 days) and long-term (3-8 months) follow-up (FIGS. 8A-8C, 9A-9C and 10, FIG. 20). Overall, toxicities were mild or absent after treatment. Notably, mice showed no reduction in body weight throughout treatment, and CBCs were normal both during and after treatment. In addition, serum levels of FLT3L, a cytokine previously shown to correlate with radiation damage in the bone marrow of human patients, did not change with treatment. Lastly, serum chemistry did not reveal any dysfunction in the kidney or liver and histological analyses of the kidney, liver, spleen, bone marrow, brain and spine revealed no treatment-related pathologies. This was highly relevant given the sensitivity of these organs to radiation-related toxicities in conventional RIT (Repetto-Llamazares, A. H. et al., PLoS One 9, e103070 (2014); Cheung, N. K. et al. J Natl Cancer Inst 77, 739-745 (1986); Subbiah, K. et al., J Nucl Med 44, 437-445 (2003)), as well as the presence of ganglioside GD2 in the mouse brain (Furukawa, K. et al., J Neurochem 105, 1057-1066 (2008)). Interestingly, despite no observations in the kidneys, some mild hyperplasia and hypertrophy were observed in the adrenal glands of mice treated with IgG-scFv-BsAb (3/3 at day 230). These adrenal gland pathologies were not observed in any other mice. However, two clinically significant treatment-related toxicities were observed: moderate to marked atrophy of the ovaries and mild to moderate chronic cystitis of the bladder (FIG. 4D, FIGS. 7A-7D and FIG. 10).

Ovarian atrophy was observed in ten mice: seven treated with IgG-scFv-BsAb 3×-3×(2/3 at 110 days, 2/3 at 155 days, 3/3 at 230 days), one mouse treated with P53-SADA-BsAb 3×-3× (1 of 9 checked) and two mice treated with P63-SADA-BsAb using the 2×-6× regimen (2 of 2 checked). Notably, this was both more frequent (7 mice vs 1-2) and of higher severity in the IgG-scFv-BsAb treated mice compared with either group of SADA-BsAb treated mice, especially among mice analyzed after 230 days (3/3 grade 4), suggesting that the ovaries atrophied over time, not immediately after radiation treatment. This toxicity was also more common among mice treated with the 2×-6× regimen compared with 1×-3× or even 3×-3×treated mice, indicating that ovarian toxicity was likely a consequence of non-specific exposure to radiation, either resulting from high doses of administered ¹⁷⁷Lu, or as a bystander effect from long-lived circulating payload in the blood pool of treated mice (i.e., DOTA bound to insufficiently cleared IgG-scFv-BsAb).

Chronic cystitis of the bladder, characterized by mild to moderate urothelial hyperplasia and variably associated with inflammatory infiltrates and fibrosis was observed in four mice, one among each of the treatment groups: IgG-scFv-BsAb 3×-3× (1/9), P53-SADA-BsAb 3×-3× (1/9), P63-SADA-BsAb 1×-3× (1/2) and P63-SADA-BsAb 2×-6× (1/2). The lack of specificity to one treatment suggested that this toxicity stemmed from the payload itself, which is known to clear into the urine. Additionally, the toxicity's presence among only a minority of treated mice was related to the amount of time ¹⁷⁷Lu remained in the bladder, which may make it improvable, if not completely avoidable. These toxicity data confirmed the safety and efficacy of SADA-BsAb for the treatment of solid tumors. Notably this regimen did not require any clearing agent and appeared to elicit both fewer and less intense toxicities to non-tumor tissues compared to 3-step IgG-PRIT.

### Example 6: P53-SADA-BsAb Ablates Established Neuroblastoma PDX Tumors

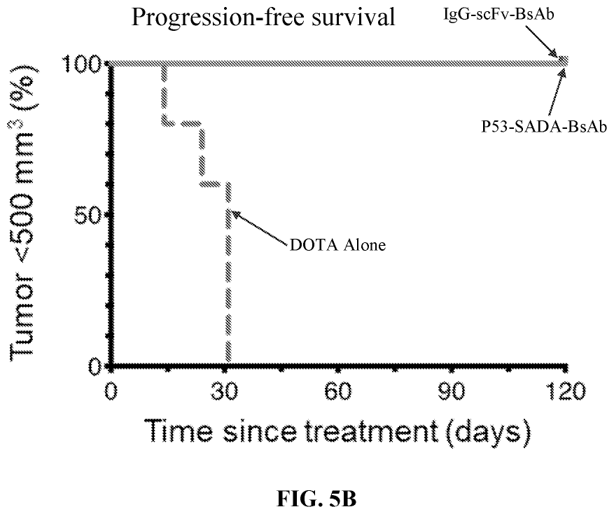

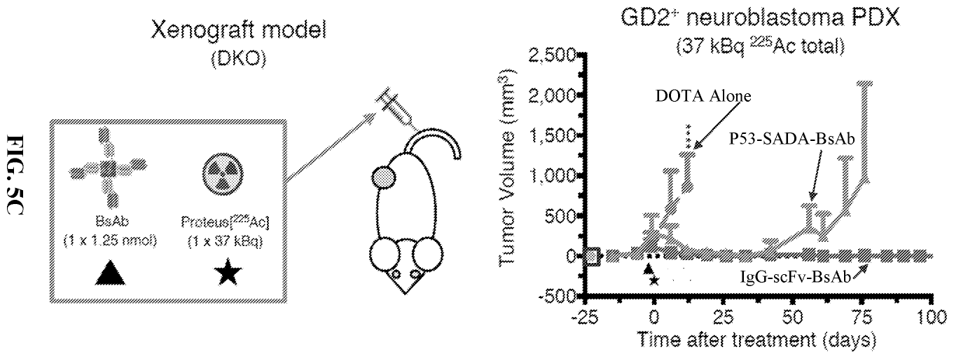

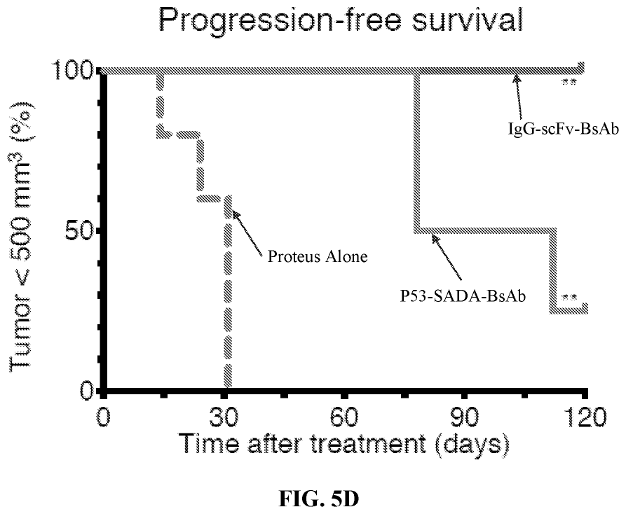

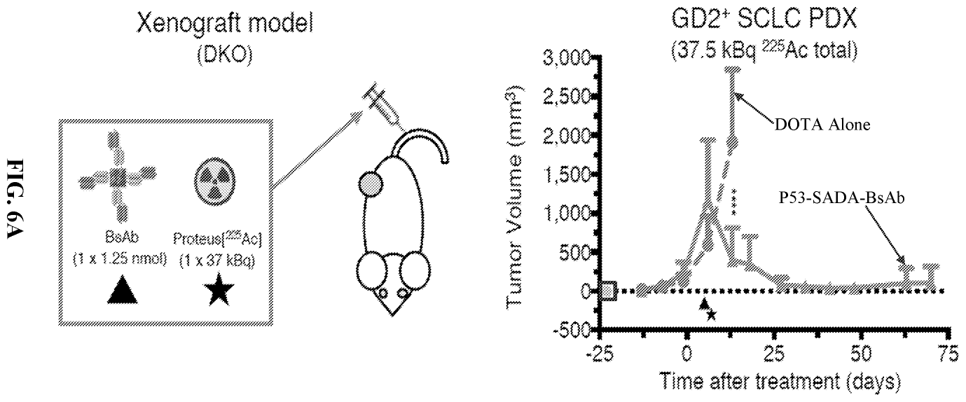

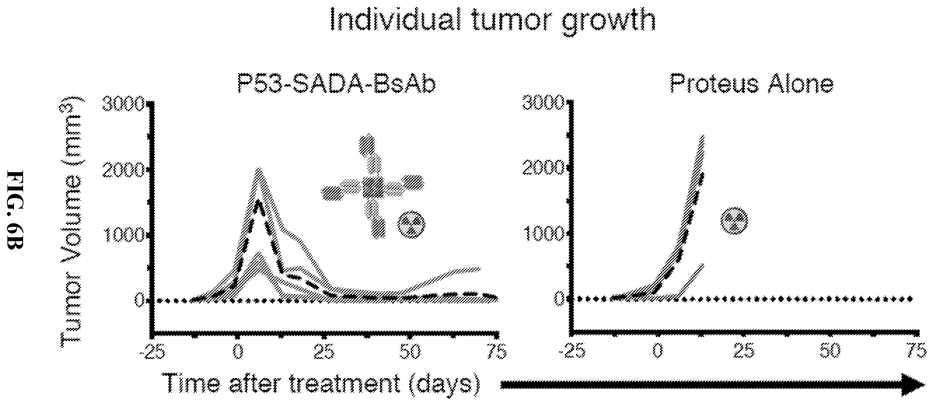

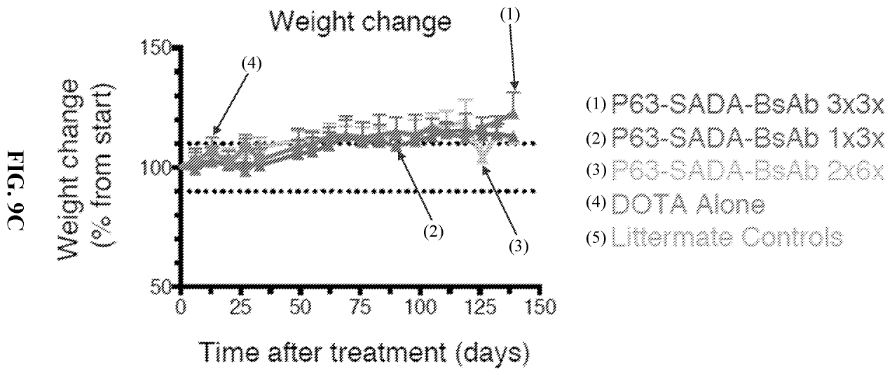

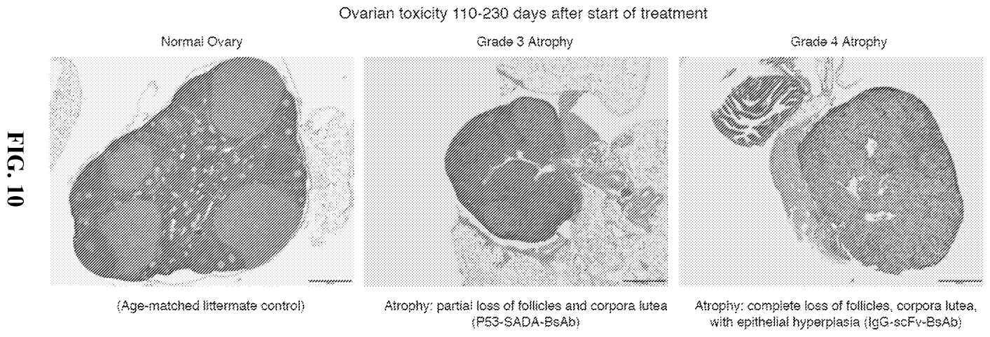

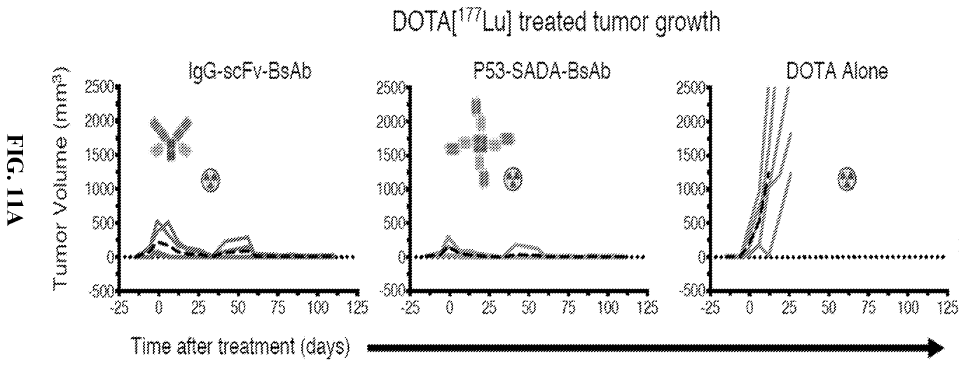

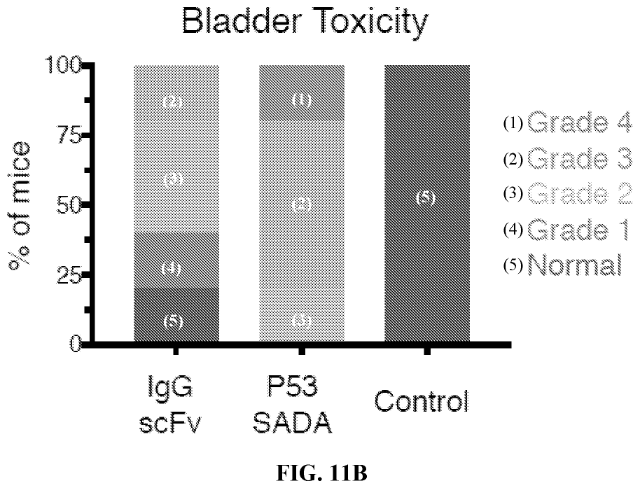

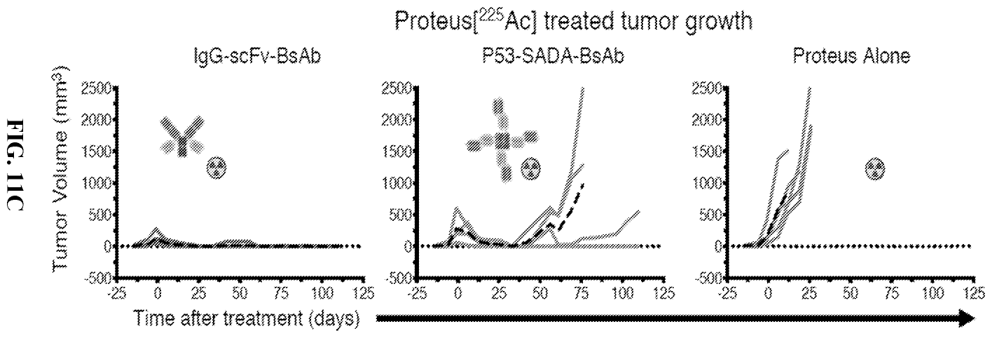

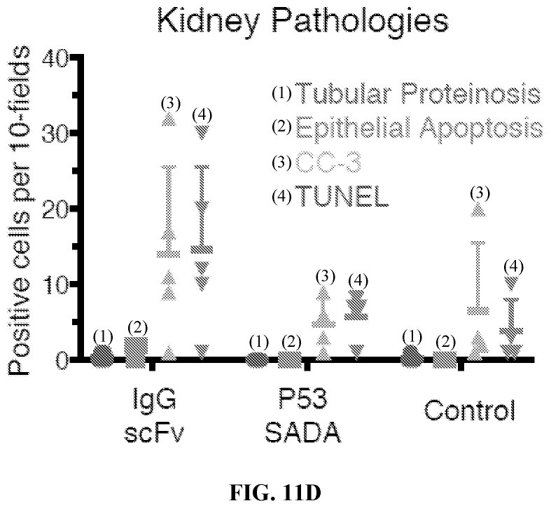

Based on the improved tumor responses observed in mice treated with additional doses of DOTA[¹⁷⁷Lu], the efficacy of P53-SADA-BsAb using a 3-fold higher dose of DOTA[¹⁷⁷Lu] payload (55.5 MBq/dose, 300 pmol) was evaluated. In this model, Rag2^{-/-} IL2rgc^{-/-} double knockout (DKO) mice bearing subcutaneous GD2⁺ patient derived xenograft (PDX) tumors were treated with either P53-SADA-BsAb or IgG-scFv-BsAb, using the same 3×-3×schedule as before (FIG. 5A). All treatment groups displayed complete responses without relapse (5/5 mice cured, in both groups), while control groups displayed uncontrolled tumor growth and were sacrificed within 30 days (FIG. 5B, FIG. 11A).

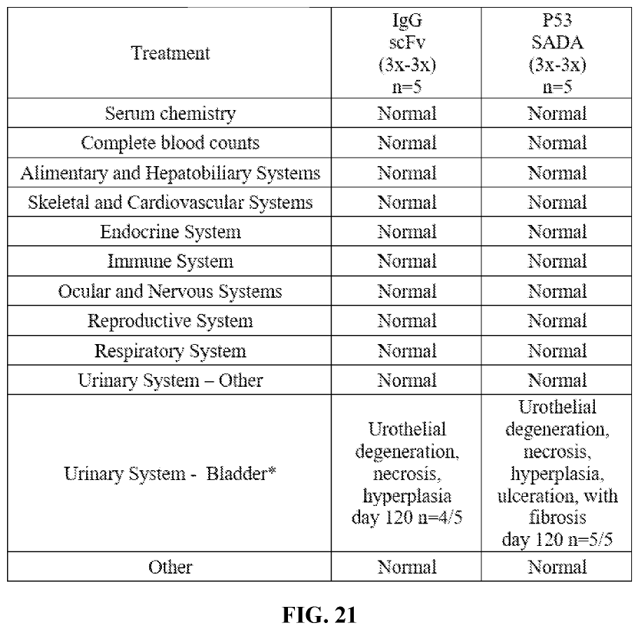

Treatment toxicities were evaluated as before, assessing measurements of short and long-term treatment related toxicities (FIG. 11B, FIGS. 12A-12B, and FIG. 21). Consistent with the previous model, neither P53-SADA-BsAb nor IgG-scFv-BsAb elicited any toxicity to the kidney, liver, bone marrow, spleen, brain or spine. However, nearly all mice displayed a severe cystitis of the bladder. Since these mice received 3-fold more payload than the previous model (FIGS. 4A-4D), the increased frequency and severity of urothelial degeneration, hyperplasia and fibrosis of the bladder suggested that this amount of payload (6,600 MBq/kg ¹⁷⁷Lu) was approaching the maximum tolerable dose to the bladder.

It is important to note, however, that serum chemistry for all treated mice was normal at the time of sacrifice, and mice did not show overt urinary dysfunction during or after treatment. This indicated that this toxicity likely developed over many weeks, which was consistent with the phenotype of radiation induced hemorrhagic cystitis in the patients (Manikandan et al., Indian J Urol 26, 159-166 (2010)). These results demonstrate that the SADA-BsAbs are useful in methods for delivering exceptionally large doses of beta-emitting radioisotope payloads to the tumor without renal, hepatic or myelotoxicities.

### Example 7: P53-SADA-BsAb can Safely Deliver Alpha-Particles to Ablate Established Neuroblastoma Tumors

A long-standing goal for radioimmunotherapy has been the safe delivery of alpha-particles to tumors, due to the higher energy release per degradation and increased rate of double strand DNA breaks. The Proteus DOTA hapten was used to deliver the alpha emitter ²²⁵Ac with 2-step SADA-PRIT (Cheal, S. et al., Journal of Nuclear Medicine 59 (2018). Due to the increased radio biological effect of alpha-particle payloads, DKO mice bearing neuroblastoma PDX tumors were treated with only a single dose of SADA-BsAb (1.25 nmol), followed by a single dose of Proteus[²²⁵Ac] 48 hours later (37 kBq, 2.4 nmol). Tumors in all treatment groups responded, including one which was over 500 mm³, while control groups showed uncontrolled tumor growth (FIGS. 5C-5D, FIG. 11C).

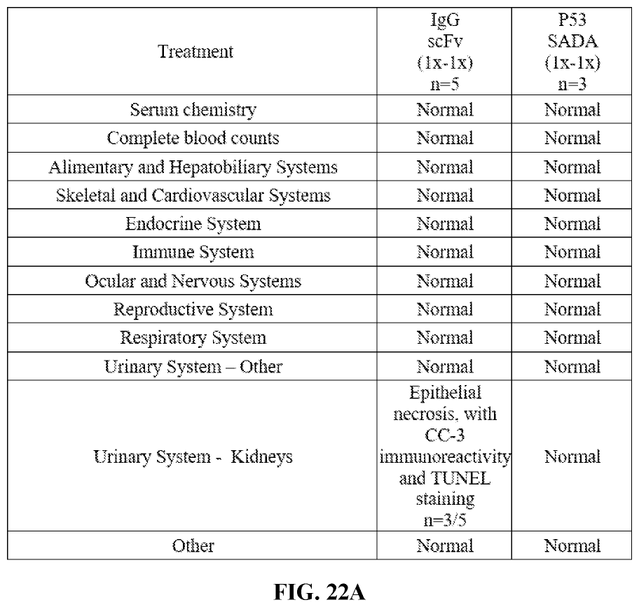

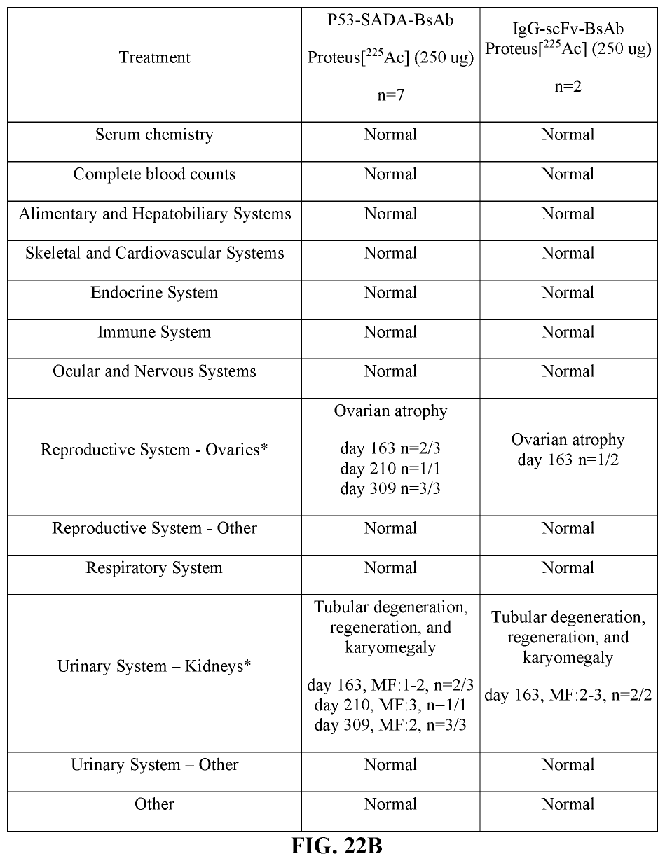

Previous attempts to deliver alpha-particles payloads to tumors have been met with numerous toxicities, especially to the kidneys and bone marrow (Jaggi, J. S. et al., J Am Soc Nephrol 16, 2677-2689, (2005)). However, treatment with P53-SADA-BsAb and Proteus[²²⁵Ac] payloads did not show any observable toxicities (FIG. 11D, FIGS. 13A-13B). CBC analysis 14 days after treatment demonstrated no myelosuppression and serum chemistry values remained normal at 120 days after treatment. In addition, histologic examinations of liver, brain, bone marrow and spleen tissues showed no evidence of radiation damage (FIG. 22A).

Interestingly, bladder toxicity was entirely absent, indicating that the bladder cystitis observed in earlier experiments came from the specific payload used (¹⁷⁷Lu), not the targeting strategy or BsAb. Since SADA-BsAb cleared primarily by renal filtration, kidneys from treated mice were thoroughly analyzed for histological changes using H&E, TUNEL staining, and cleaved caspase-3 (CC-3) immunohistochemistry. Consistent with the previous findings described herein, P53-SADA-BsAb did not elicit any observable damage to the kidneys, although some mice treated with IgG-scFv-BsAb did show mild elevation in TUNEL staining and CC-3 immunoreactivity in renal tubules, presumably due to the circulation of insufficiently cleared IgG-scFv-BsAb.

Toxicity of P53-SADA-BsAb and Proteus[²²⁵Ac] payload therapy was further assessed at 163, 210 and 309 days post treatment. As shown in FIG. 22B, no evidence of myelosuppression and radiation damage to liver, brain, bone marrow and spleen tissues was observed at up to 309 days post treatment. Moreover, animals treated with P53-SADA-BsAb showed mostly minimal to mild histopathologic abnormalities in kidneys relative to those treated with IgG-scFv-BsAbs. See FIG. 22B.

These results demonstrate that the SADA-BsAbs disclosed herein can safely deliver highly cytotoxic alpha-particle emitting payloads.

### Example 8: P53-SADA-BsAb can Ablate Established Small-Cell Lung Cancer PDX Tumors

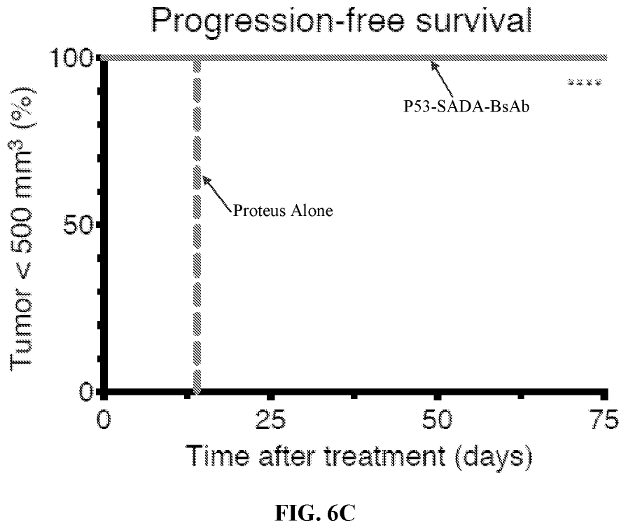

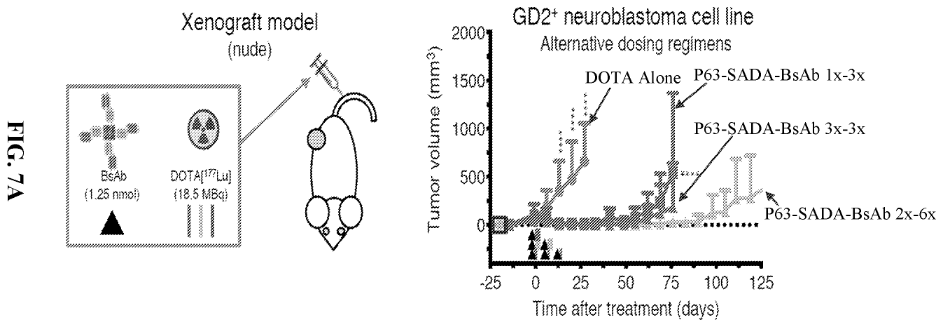

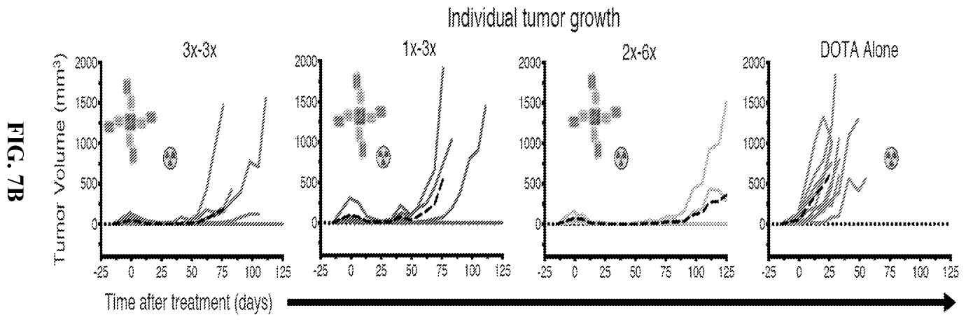

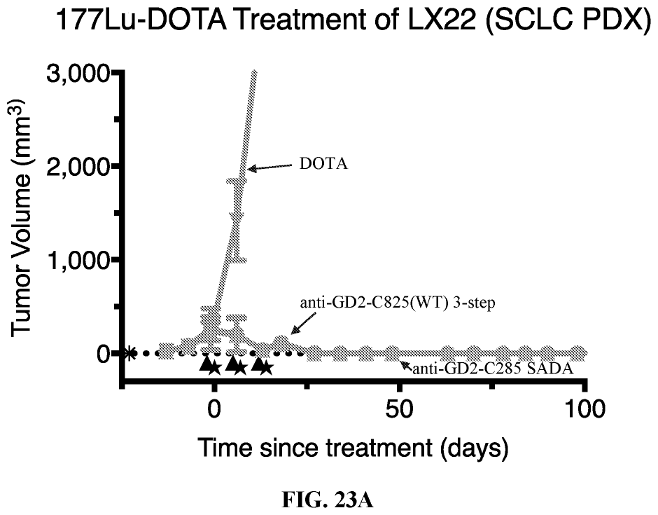

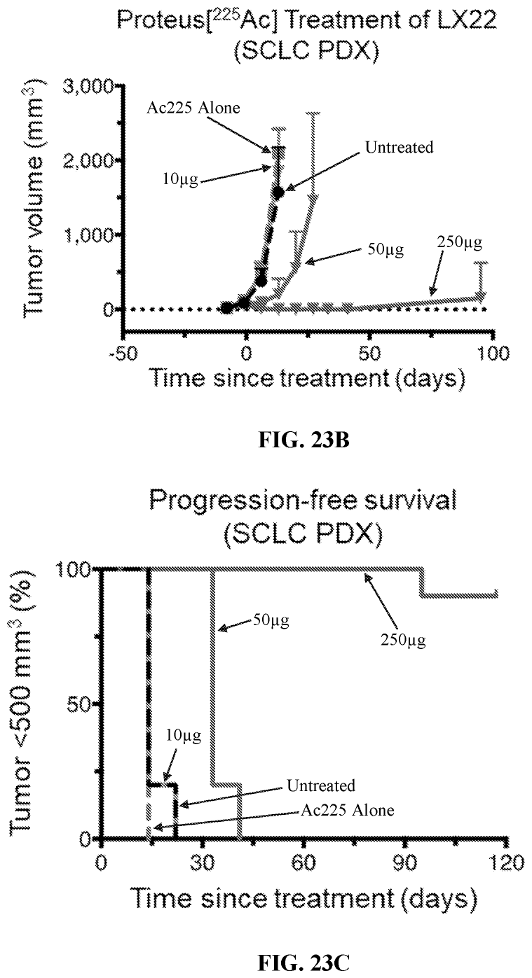

Ganglioside GD2 is expressed in a broad spectrum of human tumors besides neuroblastoma. Among them, small-cell lung cancer (SCLC) is perhaps the most difficult to treat (5-year survival of <5%). Since SCLC has previously been shown to be sensitive to radiation (Carmichael, J. et al., Eur J Cancer Clin Oncol 25, 527-534 (1989)), its response to 2-step SADA-PRIT was evaluated using DOTA [¹⁷⁷Lu] (FIG. 23) and Proteus[²²⁵Ac] payloads (FIGS. 6A-6C).

DKO mice were implanted with SCLC PDX tumors (LX22) and treated with a single cycle of SADA-BsAb (1.25 nmol) and Proteus[¹⁷⁷Lu] (37.5 kBq, 700 pmol). FIG. 23A shows that SADA-BsAb (SEQ ID NO: 27) in combination with DOTA [¹⁷⁷Lu] payload induced a robust anti-tumor response in the SCLC patient-derived xenograft (PDX) treatment model that was comparable to IgG-scFv-BsAb.

DKO mice were implanted with SCLC PDX tumors (LX22) and treated with a single cycle of SADA-BsAb (1.25 nmol) and Proteus[²²⁵Ac] (37.5 kBq, 700 pmol). Despite their massive size at the time of treatment, all treated tumors responded (FIGS. 6A-6C). Additionally, all but one tumor, the largest among them, shrank completely and durably, while tumors in control groups rapidly grew out to the maximum allowed sizes. These results demonstrate that even large masses could be effectively treated with alpha-particles, despite having a short path length compared to beta-particles. FIGS. 23B-23C demonstrate that SADA-BsAb in combination with DOTA [²²⁵Ac] payload also induced a dose-dependent anti-tumor response in the SCLC patient-derived xenograft (PDX) treatment model. A 50 μg/dose anti-GD2 SADA conjugate treated group showed low durability responses whereas mice dosed with 250 μg anti-GD2 SADA conjugate showed near complete responses (10/10).

These results demonstrate that the SADA-BsAbs of the present technology are useful in methods for treating tumors with cytotoxic payloads, especially alpha- or beta-emitting radioisotopes.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for reducing or mitigating alpha-radioimmunotherapy-associated toxicity in a subject in need thereof comprising
administering to the subject an effective amount of an anti-DOTA bispecific antigen binding fragment comprising a self-assembly disassembly (SADA) polypeptide of p53 or p63, wherein the anti-DOTA bispecific antigen binding fragment is configured to localize to a tumor expressing GD2; and
administering to the subject an effective amount of a DOTA hapten comprising an alpha particle-emitting isotope, wherein the DOTA hapten is configured to bind to the anti-DOTA bispecific antigen binding fragment.

2. The method of claim 1, wherein the subject has received or is receiving one or more cycles of alpha-radioimmunotherapy; or
wherein the alpha particle-emitting isotope is ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, or ²⁵⁵Fm; or
wherein the alpha-radioimmunotherapy-associated toxicity is toxicity to one or more organs selected from the group consisting of brain, kidney, bladder, liver, bone marrow and spleen.

3. (canceled)

4. (canceled)

5. A method for increasing the efficacy of beta-radioimmunotherapy in a subject in need thereof comprising
(I)
(a) administering to the subject an effective amount of an anti-DOTA bispecific antigen binding fragment comprising a self-assembly disassembly (SADA) polypeptide of p53 or p63, wherein the anti-DOTA bispecific antigen binding fragment is configured to localize to a tumor expressing GD2;
(b) administering to the subject a first dose of a DOTA hapten about 48 hours after administration of the anti-DOTA bispecific antigen binding fragment, wherein the DOTA hapten (i) comprises a beta particle-emitting isotope, and (ii) is configured to bind to the anti-DOTA bispecific antigen binding fragment;
(c) administering to the subject a second dose of the DOTA hapten about 24 hours after administration of the first dose of the DOTA hapten; and
(d) administering to the subject a third dose of the DOTA hapten about 24 hours after administration of the second dose of the DOTA hapten, optionally wherein the method further comprises repeating steps (a)-(d) for at least one additional cycle; or
(II)
(a) administering to the subject a first effective amount of an anti-DOTA bispecific antigen binding fragment comprising a self-assembly disassembly (SADA) polypeptide of p53 or p63, wherein the anti-DOTA bispecific antigen binding fragment is configured to localize to a tumor expressing GD2;
(b) administering to the subject a first dose of a DOTA hapten about 48 hours after administration of the first effective amount of the anti-DOTA bispecific antigen binding fragment, wherein the DOTA hapten (i) comprises a beta particle-emitting isotope, and (ii) is configured to bind to the anti-DOTA bispecific antigen binding fragment;
(c) administering to the subject a second effective amount of the anti-DOTA bispecific antigen binding fragment about 7 days after administration of the first effective amount of the anti-DOTA bispecific antigen binding fragment;
(d) administering to the subject a second dose of the DOTA hapten about 48 hours after administration of the second effective amount of the anti-DOTA bispecific antigen binding fragment;
(e) administering to the subject a third effective amount of the anti-DOTA bispecific antigen binding fragment about 7 days after administration of the second effective amount of the anti-DOTA bispecific antigen binding fragment; and
(f) administering to the subject a third dose of the DOTA hapten about 48 hours after administration of the third effective amount of the anti-DOTA bispecific antigen binding fragment.

6. (canceled)

7. (canceled)

8. The method of claim 5, wherein the first dose, the second dose, and the third dose of the DOTA hapten are different or identical; or wherein the beta particle-emitting isotope is ⁸⁶Y, ⁹⁰Y, ⁸⁹Sr, ¹⁶⁵Dy, ¹⁸⁶Re, ¹⁸⁸Re, ¹⁷⁷Lu, or ⁶⁷Cu.

9. (canceled)

10. (canceled)

11. A method for treating a GD2-associated cancer in a subject in need thereof comprising
(I)
(a) administering to the subject an effective amount of an anti-DOTA bispecific antigen binding fragment comprising a self-assembly disassembly (SADA) polypeptide of p53 or p63, wherein the anti-DOTA bispecific antigen binding fragment is configured to localize to a tumor expressing GD2;
(b) administering to the subject a first dose of a DOTA hapten about 48 hours after administration of the anti-DOTA bispecific antigen binding fragment, wherein the DOTA hapten (i) comprises a beta particle-emitting isotope or an alpha particle-emitting isotope, and (ii) is configured to bind to the anti-DOTA bispecific antigen binding fragment;
(c) administering to the subject a second dose of the DOTA hapten about 24 hours after administration of the first dose of the DOTA hapten; and
(d) administering to the subject a third dose of the DOTA hapten about 24 hours after administration of the second dose of the DOTA hapten, optionally wherein the method further comprises repeating steps (a)-(d) for at least one additional cycle; or
(II)
(a) administering to the subject a first effective amount of an anti-DOTA bispecific antigen binding fragment comprising a self-assembly disassembly (SADA) polypeptide of p53 or p63, wherein the anti-DOTA bispecific antigen binding fragment is configured to localize to a tumor expressing GD2;
(b) administering to the subject a first dose of a DOTA hapten about 48 hours after administration of the first effective amount of the anti-DOTA bispecific antigen binding fragment, wherein the DOTA hapten (i) comprises a beta particle-emitting isotope or an alpha particle-emitting isotope, and (ii) is configured to bind to the anti-DOTA bispecific antigen binding fragment;
(c) administering to the subject a second effective amount of the anti-DOTA bispecific antigen binding fragment about 7 days after administration of the first effective amount of the anti-DOTA bispecific antigen binding fragment;
(d) administering to the subject a second dose of the DOTA hapten about 48 hours after administration of the second effective amount of the anti-DOTA bispecific antigen binding fragment;
(e) administering to the subject a third effective amount of the anti-DOTA bispecific antigen binding fragment about 7 days after administration of the second effective amount of the anti-DOTA bispecific antigen binding fragment; and
(f) administering to the subject a third dose of the DOTA hapten about 48 hours after administration of the third effective amount of the anti-DOTA bispecific antigen binding fragment.

12. (canceled)

13. (canceled)

14. The method of claim 11, wherein the beta particle-emitting isotope is ⁸⁶Y, ⁹⁰Y, ⁸⁹Sr, ¹⁶⁵Dy, ¹⁸⁶Re, ¹⁸⁸Re, ¹⁷⁷Lu, or ⁶⁷Cu or wherein the alpha particle-emitting isotope is ²¹³Bi, ²¹¹At, ²²⁵Ac, ¹⁵²Dy, ²¹²Bi, ²²³Ra, ²¹⁹R, ²¹⁵Po, ²¹¹Bi, ²²¹Fr, ²¹⁷At, or ²⁵⁵Fm.

15. (canceled)

16. The method of claim 1, wherein the anti-DOTA bispecific antigen binding fragment includes a GD2-specific antigen binding domain comprising a heavy chain variable domain (V_{H}) sequence and a light chain variable domain (V_{L}) sequence of SEQ ID NO: 1 and SEQ ID NO: 5, respectively and/or wherein the anti-DOTA bispecific antigen binding fragment includes a DOTA-specific antigen binding domain comprising a heavy chain variable domain (V_{H}) sequence of SEQ ID NO: 9 or SEQ ID NO: 17, and a light chain variable domain (V_{L}) sequence of SEQ ID NO: 13 or SEQ ID NO: 18, optionally wherein the amino acid sequence of the anti-DOTA bispecific antigen binding fragment is selected from among SEQ ID NOs: 22-35 or 38-39.

17. (canceled)

18. The method of claim 16, wherein the sequence of an intra-peptide linker between the V_{H} domain sequence and the V_{L} domain sequence in the GD2-specific antigen binding domain is any one of SEQ ID NOs: 19-21; or
wherein the sequence of an intra-peptide linker between the V_{H} domain sequence and the V_{L} domain sequence in the DOTA-specific antigen binding domain is any one of SEQ ID NOs: 19-21; or
wherein the sequence of an intra-peptide linker between the GD2-specific antigen binding domain and the DOTA-specific antigen binding domain is any one of SEQ ID NOs: 19-21.

19. (canceled)

20. (canceled)

21. The method of claim 1, wherein the anti-DOTA bispecific antigen binding fragment comprises a first polypeptide chain, wherein the first polypeptide chain comprises in the N-terminal to C-terminal direction:
i. the V_{L} sequence of SEQ ID NO: 5;
ii. a flexible peptide linker comprising the amino acid sequence of any one of SEQ ID NOs: 19-21;
iii. the V_{H} sequence of SEQ ID NO: 1;
iv. a flexible peptide linker comprising the amino acid sequence of any one of SEQ ID NOs: 19-21;
v. the V_{H} sequence of SEQ ID NO: 9 or SEQ ID NO: 17;
vi. a flexible peptide linker comprising the amino acid sequence of any one of SEQ ID NOs: 19-21;
vii. the V_{L} sequence of SEQ ID NO: 13 or SEQ ID NO: 18;
viii. a flexible peptide linker sequence comprising the amino acid sequence TPLGDTTHT (SEQ ID NO: 40); and
ix. a self-assembly disassembly (SADA) polypeptide sequence of SEQ ID NO: 36 or SEQ ID NO: 37.

22. The method of claim 1, wherein the anti-DOTA bispecific antigen binding fragment comprises a first polypeptide chain, wherein the first polypeptide chain comprises in the N-terminal to C-terminal direction:
i. the V_{L} sequence of SEQ ID NO: 5;
ii. a flexible peptide linker comprising the amino acid sequence of any one of SEQ ID NOs: 19-21;
iii. the V_{H} sequence of SEQ ID NO: 1;
iv. a flexible peptide linker comprising the amino acid sequence of any one of SEQ ID NOs: 19-21;
v. the V_{L} sequence of SEQ ID NO: 13 or SEQ ID NO: 18;
vi. a flexible peptide linker comprising the amino acid sequence of any one of SEQ ID NOs: 19-21;
vii. the V_{H} sequence of SEQ ID NO: 9 or SEQ ID NO: 17;
viii. a flexible peptide linker sequence comprising the amino acid sequence TPLGDTTHT (SEQ ID NO: 40); and
ix. a self-assembly disassembly (SADA) polypeptide sequence of SEQ ID NO: 36 or SEQ ID NO: 37.

23. The method of claim 1, wherein the anti-DOTA bispecific antigen binding fragment comprises a first polypeptide chain, wherein the first polypeptide chain comprises in the N-terminal to C-terminal direction:
i. the V_{H} sequence of SEQ ID NO: 1;
ii. a flexible peptide linker comprising the amino acid sequence of any one of SEQ ID NOs: 19-21;
iii. the V_{L} sequence of SEQ ID NO: 5;
iv. a flexible peptide linker comprising the amino acid sequence of any one of SEQ ID NOs: 19-21;
v. the V_{H} sequence of SEQ ID NO: 9 or SEQ ID NO: 17;
vi. a flexible peptide linker comprising the amino acid sequence of any one of SEQ ID NOs: 19-21;
vii. the V_{L} sequence of SEQ ID NO: 13 or SEQ ID NO: 18;
viii. a flexible peptide linker sequence comprising the amino acid sequence TPLGDTTHT (SEQ ID NO: 40); and
ix. a self-assembly disassembly (SADA) polypeptide sequence of SEQ ID NO: 36 or SEQ ID NO: 37.

24. The method of claim 1, wherein the anti-DOTA bispecific antigen binding fragment comprises a first polypeptide chain, wherein the first polypeptide chain comprises in the N-terminal to C-terminal direction:
i. the V_{H} sequence of SEQ ID NO: 1;
ii. a flexible peptide linker comprising the amino acid sequence of any one of SEQ ID NOs: 19-21;
iii. the V_{L} sequence of SEQ ID NO: 5;
iv. a flexible peptide linker comprising the amino acid sequence of any one of SEQ ID NOs: 19-21;
v. the V_{L} sequence of SEQ ID NO: 13 or SEQ ID NO: 18;
vi. a flexible peptide linker comprising the amino acid sequence of any one of SEQ ID NOs: 19-21;
vii. the V_{H} sequence of SEQ ID NO: 9 or SEQ ID NO: 17;
viii. a flexible peptide linker sequence comprising the amino acid sequence TPLGDTTHT (SEQ ID NO: 40); and
ix. a self-assembly disassembly (SADA) polypeptide sequence of SEQ ID NO: 36 or SEQ ID NO: 37.

25. (canceled)

26. The method of claim 1, wherein the subject suffers from or is diagnosed as having a GD2-associated cancer, optionally wherein the GD2-associated cancer is neuroblastoma, melanoma, soft tissue sarcoma, brain tumor, osteosarcoma, small-cell lung cancer, breast cancer, or retinoblastoma, optionally wherein the soft tissue sarcoma is liposarcoma, fibrosarcoma, malignant fibrous histiocytoma, leimyosarcoma, or spindle cell sarcoma.

27. (canceled)

28. (canceled)

29. The method of claim 1, wherein the DOTA hapten is selected from the group consisting of DOTA, Proteus-DOTA, DOTA-Bn, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, and Ac-D-Lys(DOTA)-D-Tyr-D-Lys(DOTA)-D-Lys(Tscg-Cys)-NH₂.

30. The method of claim 1, wherein the administration of the anti-DOTA bispecific antigen binding fragment results in decreased renal apoptosis in the subject compared to a GD2-associated cancer patient that has been treated with an anti-DOTA×anti-GD2 IgG-scFv-BsAb.

31. The method of claim 1, wherein the administration of the anti-DOTA bispecific antigen binding fragment results in reduced immunogenicity in the subject compared to a GD2-associated cancer patient that has been treated with an anti-DOTA×anti-GD2 IgG-scFv-BsAb.

32. The method of claim 1, wherein the administration of the anti-DOTA bispecific antigen binding fragment results in decreased severity of ovarian atrophy in the subject compared to a GD2-associated cancer patient that has been treated with an anti-DOTA×anti-GD2 IgG-scFv-BsAb.

33. The method of claim 1, wherein the administration of the anti-DOTA bispecific antigen binding fragment results in prolonged remission in the subject compared to a GD2-associated cancer patient that has been treated with an anti-DOTA×anti-GD2 IgG-scFv-BsAb.

34. The method of claim 30, wherein the anti-DOTA×anti-GD2 IgG-scFv-BsAb comprises (a) a GD2-specific antigen binding domain comprising a heavy chain variable domain (V_{H}) sequence and a light chain variable domain (V_{L}) sequence of SEQ ID NO: 1 and SEQ ID NO: 5, respectively, and (b) a DOTA-specific antigen binding domain comprising a heavy chain variable domain (V_{H}) sequence of SEQ ID NO: 9 or SEQ ID NO: 17, and a light chain variable domain (V_{L}) sequence of SEQ ID NO: 13 or SEQ ID NO: 18.

35. The method of claim 1, wherein the administration of the anti-DOTA bispecific antigen binding fragment results in decreased renal apoptosis, decreased severity of ovarian atrophy, and/or prolonged remission in the subject compared to a control GD2-associated cancer patient that does not receive the anti-DOTA bispecific antigen binding fragment.


SEQUENCE LISTING (42 sequences)

SEQ ID NO: 1 - protein, 119 aa, Artificial Sequence
  Description of Artificial Sequence Synthetic polypeptide
QVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSS

SEQ ID NO: 2 - protein, 5 aa, Artificial Sequence
  Description of Artificial Sequence Synthetic peptide
NYGVH

SEQ ID NO: 3 - protein, 16 aa, Artificial Sequence
  Description of Artificial Sequence Synthetic peptide
VIWAGGITNYNSAFMS

SEQ ID NO: 4 - protein, 11 aa, Artificial Sequence
  Description of Artificial Sequence Synthetic peptide
RGGHYGYALDY

SEQ ID NO: 5 - protein, 105 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKR

SEQ ID NO: 6 - protein, 11 aa, Artificial Sequence
  Description of Artificial Sequence Synthetic peptide
KASQSVSNDVT

SEQ ID NO: 7 - protein, 7 aa, Artificial Sequence
  Description of Artificial Sequence Synthetic peptide
SASNRYS

SEQ ID NO: 8 - protein, 6 aa, Artificial Sequence
  Description of Artificial Sequence Synthetic peptide
QQDYSS

SEQ ID NO: 9 - protein, 119 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSS

SEQ ID NO: 10 - protein, 5 aa, Artificial Sequence
  Description of Artificial Sequence Synthetic peptide
DYGVH

SEQ ID NO: 11 - protein, 16 aa, Artificial Sequence
  Description of Artificial Sequence Synthetic peptide
VIWSGGGTAYNTALIS

SEQ ID NO: 12 - protein, 11 aa, Artificial Sequence
  Description of Artificial Sequence Synthetic peptide
RGSYPYNYFDA

SEQ ID NO: 13 - protein, 110 aa, Artificial Sequence
  Description of Artificial Sequence Synthetic polypeptide
QAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 14 - protein, 14 aa, Artificial Sequence
  Description of Artificial Sequence Synthetic peptide
GSSTGAVTASNYAN

SEQ ID NO: 15 - protein, 7 aa, Artificial Sequence
  Description of Artificial Sequence Synthetic peptide
GHNNRPP

SEQ ID NO: 16 - protein, 9 aa, Artificial Sequence
  Description of Artificial Sequence Synthetic peptide
ALWYSDHWV

SEQ ID NO: 17 - protein, 119 aa, Artificial Sequence
  Description of Artificial Sequence Synthetic polypeptide
HVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSS

SEQ ID NO: 18 - protein, 110 aa, Artificial Sequence
  Description of Artificial Sequence Synthetic polypeptide
QAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLG

SEQ ID NO: 19 - protein, 15 aa, Artificial Sequence
  Description of Artificial Sequence Synthetic peptide
GGGGSGGGGSGGGGS

SEQ ID NO: 20 - protein, 20 aa, Artificial Sequence
  Description of Artificial Sequence Synthetic peptide
GGGGSGGGGSGGGGSGGGGS

SEQ ID NO: 21 - protein, 30 aa, Artificial Sequence
  Description of Artificial Sequence Synthetic polypeptide
GGGGSGGGGSGGGGSGGGGSGGGGSGGGGS

SEQ ID NO: 22 - protein, 559 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGA

SEQ ID NO: 23 - protein, 581 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 24 - protein, 599 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 25 - protein, 566 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 26 - protein, 584 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 27 - protein, 596 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 28 - protein, 614 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 29 - protein, 559 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGA

SEQ ID NO: 30 - protein, 581 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGAPHHHHHH

SEQ ID NO: 31 - protein, 599 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 32 - protein, 566 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGAPHHHHHH

SEQ ID NO: 33 - protein, 584 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 34 - protein, 596 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGAPHHHHHH

SEQ ID NO: 35 - protein, 614 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 36 - protein, 39 aa, Homo sapiens
KPLDGEYFTLQIRGRERFEMFRELNEALELKDAQAGKEP

SEQ ID NO: 37 - protein, 57 aa, Homo sapiens
RSPDDELLYLPVRGRETYEMLLKIKESLELMQYLPQHTIETYRQQQQQQHQHLLQKQ

SEQ ID NO: 38 - protein, 607 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGA

SEQ ID NO: 39 - protein, 589 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGA

SEQ ID NO: 40 - protein, 9 aa, Artificial Sequence
  Description of Artificial Sequence Synthetic peptide
TPLGDTTHT

SEQ ID NO: 41 - protein, 6 aa, Artificial Sequence
  Description of Artificial Sequence Synthetic 6xHis tag
HHHHHH

SEQ ID NO: 42 - protein, 150 aa, Artificial Sequence
  Description of Artificial Sequence Synthetic polypeptide
  See specification as filed for detailed description of substitutions and preferred embodiments
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS